(19) United States
(12) Patent Application Publication
(10) Pub. No.: US 2023/0082929 A1
(43) Pub. Date: Mar. 16, 2023

(54) METHODS OF GENERATING AND USING SENESCENT-INDUCED CELLS FOR TREATMENT OF CANCER AND COMPOSITIONS RELATING THERETO

(71) Applicant: The Wistar Institute of Anatomy and Biology, Philadelphia, PA (US)

(72) Inventors: Bo Zhao, Philadelphia, PA (US); Rugang Zhang, Elkins Park, PA (US)

(21) Appl. No.: 17/760,463

(22) PCT Filed: Feb. 12, 2021
(86) PCT No.: PCT/US21/17859

§ 371 Date: Aug. 10, 2022

Related U.S. Application Data
(60) Provisional application No. 62/976,020, filed on Feb. 13, 2020.

(51) Int. Cl. A61K 35/13; A61K 39/395; A61K 45/06; C12N 5/09; A61P 35/00; A61P 15/00

(52) U.S. Cl. CPC A61K 35/13; A61K 39/39558; A61K 45/06; C12N 5/0693; A61P 35/00; A61P 15/00

(57) ABSTRACT

Compositions and methods for treating cancer in a subject in need thereof is provided. In certain embodiments, the method includes administering therapy-induced senescent (TIS) cells and an immune checkpoint inhibitor to the subject. Also provided are compositions comprising therapy-induced senescent (TIS) cells.

### STATEMENT OF GOVERNMENT SUPPORT

This invention was made with government support under grant numbers R01CA160331, P01AG031862, and CA010815 awarded by the National Institutes of Health. The government has certain rights in the invention.

### BACKGROUND OF THE INVENTION

The immune system is critical in fighting cancer. However, at certain times, the immune system has trouble finding and/or recognizing the cancer cells that should be eliminated. These cancer cells are “cold” to the immune system, i.e., difficult to be located and destroyed.

Cellular senescence is a bona fide tumor suppression mechanism that can be induced by a number of stresses including chemotherapeutics such as cisplatin (Herranz and Gil, 2018). Therapy-induced senescence is tumor suppressive by triggering a stable cell growth arrest (Herranz and Gil, 2018). Senescent cells also have non-cell autonomous activities exemplified by secretion of inflammatory cytokines and chemokines, which is termed the senescence-associated secretory phenotype (SASP)(Coppe et al., 2008).

Immune checkpoint blockades (ICBs) such as monoclonal antibodies targeting the PD-1/PD-L1 axis have demonstrated striking clinical benefit in several cancer types (Darvin et al., 2018). However, despite this important advance, the majority of cancers show unacceptably low response rates to ICB (O'Donnell et al., 2017). Therefore, new therapeutic strategies are urgently needed to expand the utility of ICBs through sensitizing ICBs resistant tumors. Ovarian cancer remains the most lethal gynecological malignancy in the developed world. Tumor-infiltrating lymphocytes positively correlate with ovarian cancer patient survival, which is recognized as a predictive biomarker for immunotherapy and chemotherapy responses (Zhang et al., 2003). Notably, CD8+ T cells are important antitumor effectors in ovarian cancer (Sato et al., 2005). However, objective response rates to ICB in ovarian cancer range from 5.9 to 15% (Wang et al., 2019). Therefore, sensitizing ICB resistant ovarian cancer to ICB remains an unmet clinical need. Harnessing the SASP to help transform a “cold” tumor to a “hot” tumor would appear to be an effective strategy in targeting elusive cancer cells. However, accumulating evidence shows that senescent cells can have deleterious effects on the tissue microenvironment. The most significant of these effects is the acquisition of the SASP which turns senescent fibroblasts into proinflammatory cells that have the ability to promote tumor progression.

Therefore, what is needed is a method to harness the power of the SASP in turning cancer cells from “cold” to “hot” without the detrimental effects associated with the phenotype.

### SUMMARY OF THE INVENTION

Provided herein are compositions and methods for treating cancer in a subject in need thereof. The compositions and methods described herein harness the power of the SASP to “light up” cancer cells, making them visible to the immune system. As described herein, ex vivo therapy-induced senescent (TIS) cells are able to home to the residual cancer cells from which the TIS cells originated, lighting up residual cancer and allowing the immune system to target and destroy these cells.

In one aspect, a method of treating cancer in a subject in need thereof is provided. In one embodiment, the method includes administering therapy-induced senescent (TIS) cells and an immune checkpoint inhibitor to the subject. In another embodiment, the method includes obtaining cancer cells from the subject; treating the cancer cells with a chemotherapeutic agent or radiation and a TOP inhibitor to induce senescence; optionally, confirming senescence and/or sorting senescent cells from non-senescent cells; administering the senescent cells to the subject; and administering a checkpoint inhibitor to the subject. In certain embodiments, the checkpoint inhibitor is a PD-1 or PD-L1 inhibitor or CTLA4 inhibitor.

In another aspect, a pharmaceutical composition is provided. The composition includes therapy induced senescent cells, as described herein, and a pharmaceutically acceptable carrier, diluent, or excipient.

In another aspect, provided herein is a pharmaceutical composition produced by a method that includes the following: obtaining cancer cells from a subject; treating the cancer cells ex vivo with a chemotherapeutic agent and an inhibitor of TOP1, TOP2, or both to produce therapy induced senescent (TIS) cells; and optionally, confirming senescence and/or sorting senescent cells from non-senescent cells.

Other aspects and advantages of the invention will be readily apparent from the following detailed description of the invention.

### BRIEF DESCRIPTION OF THE DRAWINGS

FIG. 1A-FIG. 1I show HMGB2 is required for cGAS' localization into CCF (cytoplasmic chromatin fragments) during senescence. (FIG. 1A) OVCAR3 cells expressing GFP-tagged cGAS were induced to senesce by cisplatin, and imaged under a confocal microscope. cGAS-GFP, γH2AX and HMGB2 co-localized CCF are indicated by arrows. (FIG. 1B) Expression of HMGB2, cGAS and a loading control β-actin in the indicated parental and two independent HMGB2 knockout OVCAR3 clones determined by immunoblot. (FIG. 1C-FIG. 1D) Representative images (FIG. 1C) and quantification (FIG. 1D) of endogenous cGAS localization into γH2AX positive CCF in senescent parental and HMGB2 knockout OVCAR3 cells with the indicated treatment. Arrows point to CCF. (FIG. 1E-FIG. 1F) Representative images (FIG. 1E) and quantification (FIG. 1F) of cGAS-GFP localization into γH2AX positive CCF in senescent parental and HMGB2 knockout OVCAR3 cells with the indicated treatment. Arrows point to CCF. FIG. 1G-FIG. 1H) Quantification of cGAS (FIG. 1G) or cGAS-GFP (FIG. 1H) localization into CCF in senescent primary IMR90 cells induced by oncogenic RAS with or without HMGB2 knockdown. (FIG. 1I) The secretion of soluble factors under the indicated conditions were detected by antibody arrays. The heatmap indicates the fold change (FC) in comparison to the control (Ctrl) or RAS-induced senescent IMR90 cells. Relative expression levels per replicate and average fold change differences are shown. Data represent mean±s.e.m. n=3 biologically independent experiments unless otherwise stated. Scale bar=10 μm. P values were calculated using a two-tailed t-test.

FIG. 2A-FIG. 2K show cGAS activation requires TOP1cc during senescence. (FIG. 2A) Slot blot analysis of total TOP1 proteins in CCF purified from senescent IMR90 cells induced by the indicated treatment with or without HMGB2 knockdown. (FIG. 2B-FIG. 2C) IMR90 cells induced to senesce by etoposide or oncogenic RAS were stained for TOP1cc and γH2AX (FIG. 2B) and percentages of γH2AX positive CCF positive for TOP Ice were quantified (FIG. 2C). CCF are indicated by arrows. (FIG. 2D) Slot blot analysis of TOP1cc levels in CCF purified from senescent IMR90 cells induced by the indicated treatment with or without HMGB2 knockdown. (FIG. 2E) Slot blot analysis of TOP1cc levels in CCF purified from the indicated IMR90 cells treated with or without TOP1 inhibitor Camptothecin (CPT). (FIG. 2F-FIG. 2G) Examination of STING dimerization (FIG. 2F) or 2′3′-cGAMP levels (FIG. 2G) in the indicated cells. (FIG. 2H) Quantification of cGAS localization into CCF in the indicated IMR90 cells. (FIG. 2I) The secretion of soluble factors under the indicated conditions were detected by antibody arrays. The heatmap indicates the fold change (FC) in comparison to the control (Ctr) or RAS condition. Relative expression levels per replicate and average fold change differences are shown. (FIG. 2J-FIG. 2K) IMR90 cells were transfected with chromatin fragments isolated from IMR90 cells with or without CPT treatment. Benzonase was used to digest chromatin into fragments. Representative images (FIG. 2J) and quantification (FIG. 2K) of cGAS and TOP1cc co-localization in the transfected IMR90 cells. Arrows point to cGAS foci induced by the transfected chromatin fragments without or with TOP Ice. Data represent mean±s.e.m. n=3 biologically independent experiments unless otherwise stated. Scale bar=10 μm. P values were calculated using a two-tailed t-test.

FIG. 3A-FIG. 3D show HMGB2 stabilizes TOP1cc. (FIG. 3A-FIG. 3B) IMR90 cells with or without HMGB2 knockdown treated with CPT were analyzed for TOP1cc levels at the indicated time points by slot blot (FIG. 3A). Fold changes in TOP Ice levels at the indicated time points were quantified (n=3) (FIG. 3B). (FIG. 3C-FIG. 3D) IMR90 cells with or without HMGB2 knockdown treated with CPT for 60 mins and released from the treatment for 60 or 120 mins were analyzed for TOP Ice levels by slot blot (FIG. 3C). Fold changes in TOP1cc levels at the indicated release time points were quantified (n=3) (FIG. 3D). Data represent mean±s.e.m. n=3 biologically independent experiments. P values were calculated using a two-tailed t-test.

FIG. 4A-FIG. 4E show TOP1cc enhances dsDNA recognition by cGAS. (FIG. 4A) Co-immunoprecipitation analysis of cGAS and TOP1 in control and RAS-induced senescent IMR90 cells. An isotype matched IgG was used as a negative control. (FIG. 4B) Co-Immunoprecipitation analysis of cGAS and TOP1 in control and RAS-induced senescent IMR90 cells with or without HMGB2 knockdown. Note that addition of (ISD)₂ dsDNA into the supernatant rescues the interaction between cGAS and TOP1 in HMGB2 knockdown senescent IMR90 cells. (FIG. 4C) Electrophoretic mobility shift analysis of TOP1 wide-type and TOP1 Y723F mutant proteins. (FIG. 4D) Electrophoretic mobility shift analysis shows that wild-type TOP1, but not the mutant TOP1 Y723, enhances the binding of cGAS to dsDNA. (FIG. 4E) A proposed model as described in the text. Data represent mean±s.e.m. P values were calculated using a two-tailed t-test.

FIG. 5A-FIG. 5G show HMGB2-TOP1cc-cGAS axis determines response to immune checkpoint blockade. (FIG. 5A) Mouse ID8-Defb29 Vegf-a ovarian cancer cells expressing doxycycline (DOX) inducible shHMGB2 with or without DOX induction were analyzed for expression of HMGB2, cGAS and a loading control β-actin by immunoblot. (FIG. 5B) Representative bioluminescence images of mice in the indicated treatment groups at the end of experiments. (FIG. 5C) Quantification of tumor growth based on luciferase bioluminescence in the indicated treatment groups at the indicated time points (n=5 biologically independent mice per group). (FIG. 5D) Expression of the indicated SASP factors in tumor cells sorted by FACS from ascites formed in mice from the indicated groups determined by qRT-PCR (n=4 biologically independent mice per group). (FIG. 5E) After stopping the treatment, the mice from the indicated groups were followed for survival. Shown is the Kaplan-Meier survival curves (n=5 biologically independent mice per group). (FIG. 5F-FIG. 5G) At the end of treatment, percentage of CD69 positive cells in CD8 positive T cells (FIG. 5F) and IFNγ positive cells in CD8 positive T cells (FIG. 5G) was assessed by flow cytometry in the peritoneal wash collected from mice in the indicated treatment groups (n=5 biologically independent mice per group). Data represent mean s.e.m. P values were calculated using a two-tailed t-test except for FIG. 5E by log-rank (Mantel-Cox) test.

FIG. 6A-FIG. 6E show HMGB2 is not required for formation of γH2AX positive CCF during therapy-induced senescence of ovarian cancer cells. (FIG. 6A) Parental and HMGB2 knockout OVCAR3 ovarian cancer cells were treated with Etoposide or Cisplatin to induce senescence. Expression of Cyclin A and a loading control β-actin in the indicated cells was examined by immunoblot. (FIG. 6B-FIG. 6B) Representative images (FIG. 6B) and quantification (FIG. 6C) of SA-β-Gal staining of OVCAR3 cells with the indicated treatments. (FIG. 6D) Co-staining of HMGB2 and γH2AX in control and senescent OVCAR3 cells induced by in the indicated treatments. Arrows point to CCF. (FIG. 6E) Quantification of γH2AX positive CCF formation in the indicated control and therapy-induced senescent OVCAR3 cells. Data represent mean±s.e.m. Scale bar=100 μm in FIG. 6B. Scale bar=10 μm in FIG. 6D.

FIG. 7A-FIG. 7H show HMGB2 is required for cGAS' localization into CCF. (FIG. 7A) Expression of HMGB2, cGAS and a loading control β-actin in IMR90 cells expressing the indicated shHMGB2s or control was determined by immunoblot. (FIG. 7B-FIG. 7C) Representative images (FIG. 7B) and quantification (FIG. 7C) of SA-β-Gal staining in IMR90 cells expressing oncogenic RAS or treated with Etoposide to induce senescence. (FIG. 7D-FIG. 7E) Expression of the indicated proteins in IMR90 cells induced to senesce by oncogenic RAS (FIG. 7D) or Etoposide (FIG. 7E) with or without HMGB2 knockdown was determined by immunoblot. (FIG. 7F) cGAS-GFP localization into γH2AX positive CCF in Etoposide-induced senescent IMR90 cells with or without HMGB2 knockdown. Arrows point to CCF. (FIG. 7G) Quantification of γH2AX positive CCF formation in the indicated control and RAS-induced senescent IMR90 cells. (FIG. 7H) Expression of the indicated SASP factors in the indicated IMR90 cells determined by qRT-PCR. Data represent mean s.e.m. Scale bar=100 μm in FIG. 7B. Scale bar=10 μm in FIG. 7F. P values were calculated using a two-tailed t-test.

FIG. 8A-FIG. 8G show HMGB2 inhibition increases TOP1 levels in CCF. (FIG. 8A) Schematics of the protocol used for CCF purification. (FIG. 8B) Agarose gel electrophoresis of DNA isolated from the purified CCF in senescent IMR90 cells induced by Etoposide. (FIG. 8C) Purified CCF from Etoposide-induced senescent IMR90 cells visualized by DAPI staining. (FIG. 8D) Purified CCF were transfected into IMR90 cells and the transfected cells were stained with DAPI to visualize the transfected CCF. Arrows point to transfected CCF. (FIG. 8E) Expression of the indicated SASP factors in the CCF transfected IMR90 cells was determined by qRT-PCR. Lipo2000 transfection reagent was used as a negative control. (FIG. 8F-FIG. 8G), Schematics of Stable Isotope Labeling by Amino acids in Cell culture (SILAC) combined with mass spectrometry analysis used to identify HMGB2-dependent changes in composition of CCF purified from etoposide induced senescent IMR90 cells (FIG. 8F). Fold changes of the list of proteins implicated in nucleosome and chromosome-related functionality identified from the analysis in two technical repeats of LC-MS/MS analyses (FIG. 8G). Data represent mean±s.e.m. Scale bar=20 μm. P values were calculated using a two-tailed t-test.

FIG. 9A-FIG. 9E show TOP1 knockdown suppresses SASP gene expression. (FIG. 9A) Co-staining TOP1 and γH2AX in control and the indicated senescent IMR90 cells. Arrows point to CCF. (FIG. 9B) Co-staining TOP1 and γH2AX in control and the indicated senescent OVCAR3 cells. Arrows point to CCF. (FIG. 9C) Slot blot analysis of TOP1 proteins in CCF purified from the indicated senescent OVCAR3 cells with or without HMGB2 knockout. (FIG. 9D-FIG. 9E) Expression of TOP1, HMGB2 and a loading control β-actin in IMR90 cells expressing shTOP1 or control was determined by immunoblot (FIG. 9D). Expression of the indicated SASP factors was determined by qRT-PCR analysis (FIG. 9E). Data represent mean±s.e.m. Scale bar=10 μm. P values were calculated using a two-tailed t-test.

FIG. 10A-FIG. 10J show TOP1cc is required for cGAS' localization into CCF and SASP gene expression. (FIG. 10A) Slot blot analysis of TOP1cc levels in CCF purified from the indicated control or senescent OVCAR3 cells with or without HMGB2 knockout. (FIG. 10B) Rescue of the decrease in TOP1cc levels in CCF purified from HMGB2 knockout Etoposide-induced senescent OVCAR3 cells by Camptothecin (CPT) treatment. (FIG. 10C-FIG. 10D) Quantification of cGAS-GFP (FIG. 10C) or endogenous cGAS (FIG. 10D) localization into CCF in the indicated senescent cells induced by oncogenic RAS in IMR90 cells or Etoposide treatment in OVCAR3 cells. (FIG. 10E) Co-staining TOP1cc, cGAS and γH2AX in CCF of the indicated senescent OVCAR3 cells. Arrows point to CCF. (FIG. 10F) The percentages of the indicated colocalization in the indicated senescent cells. (FIG. 10G) Expression of ISG15 in the indicated IMR90 cells determined by qRT-PCR (n=4). (FIG. 10H) Schematics of isolation and transfection of CPT-induced TOP1cc positive chromatin fragments. (FIG. 10I-FIG. 10J) Expression of cGAS, TOP1, HMGB2 and a loading control β-actin in IMR90 cells expressing the indicated shcGAS or control was determined by immunoblot (FIG. 10I). Expression of the indicated SASP genes was determined by qRT-PCR analysis (n=4) (FIG. 10J). Data represent mean±s.e.m. P values were calculated using a two-tailed t-test.

FIG. 11A-FIG. 11E show binding of cGAS to dsDNA determined by electrophoretic mobility shift assay. (FIG. 11A) GST pull down assay for the co-incubated purified His-tagged TOP1 using GST or GST-tagged cGAS. The pull down product was subjected to immunoblot analysis using an anti-TOP1 antibody. (FIG. 11B) Co-staining of TOP1 and γH2AX in CCF in control and the indicated senescent IMR90 cells with or without DNase I digestion. Arrows point to CCF. (FIG. 11C) The integrated intensity of the indicated markers in CCF of the indicated cells. (FIG. 11D) Coomassie Blue staining the indicated purified proteins used for electrophoretic mobility shift assay. (FIG. 11E) Dose-dependent dsDNA binding ability of cGAS protein.

FIG. 12A-FIG. 12J show HMGB2-TOP1cc-cGAS axis is conserved in cisplatin-induced senescent mouse ID8-Defb29/Vegf-a ovarian cancer cells. (FIG. 12A-FIG. 12B) Representative images (FIG. 12A) and quantification (FIG. 12B) of SA-β-gal staining of ID8-Defb29 Vegf-a cells treated without or with Cisplatin to induce senescence. (FIG. 12C-FIG. 12D) Co-staining of TOP1 (FIG. 12C) or TOP1cc (FIG. 12D) and γH2AX in CCF of cisplatin-induced senescent ID8-Defb29 Vegf-a cells. Arrows point to CCF. (FIG. 12E) Quantification of γH2AX positive CCF formation in the indicated control and cisplatin-induced senescent ID8-Defb29 Vegf-a cells with or without inducible HMGB2 knockdown. (FIG. 12F) Quantification of cGAS localization into CCF in cisplatin-induced senescent ID8-Defb29 Vegf-a cells with or without inducible HMGB2 knockdown. (FIG. 12G-FIG. 12H) Slot blot analysis of TOP1 (FIG. 12G) and TOP1cc (FIG. 12H) levels in CCF purified from senescent ID8 cells with or without HMGB2 knockdown. (FIG. 12I) Rescue of the decrease in TOP1cc levels in CCF purified from HMGB2 knockdown Cisplatin-induced senescent ID8-Defb29 Vegf-a cells by Camptothecin (CPT) treatment. (FIG. 12J) Quantification of cGAS localization into CCF in the indicated Cisplatin-induced senescent ID8-Defb29 Vegf-a cells. Data represent mean s.e.m. Scale bar=10 μm. P values were calculated using a two-tailed t-test.

FIG. 13A-FIG. 13E show anti-PD-L1 and CPT combination does not affect body weight of the tumor bearing mice. (FIG. 13A) Expression of the indicated SASP genes in the indicated ID8-Defb29/Vegf-a cells determined by qRT-PCR (n=4). (FIG. 13B) Body weight analysis of mice from the indicated treatment groups during the treatment period (n=5 mice per group). (FIG. 13C) The gating strategy used for determining the indicated immune cell populations. (FIG. 13D-FIG. 13E) At the end of treatment, percentage of CD69 positive CD4 T cells (FIG. 13D) and Granzyme B positive CD8 T cells (FIG. 13E) was assessed by flow cytometry in the peritoneal wash collected from mice in the indicated treatment groups (n=5 mice per group). Data represent mean±s.e.m.

FIG. 14A-FIG. 14H show isolation of SASP-boosted therapy-induced senescent cells. (FIG. 14A-FIG. 14C) UPK10 cells were treated with 10 mM cisplatin for three days. After three days of release, cells were stained for SA-β-gal activity (FIG. 14A) and percentage of SA-β-gal positive cells were quantified (FIG. 14B). Expression of the indicated proteins was also examined by immunoblot in the indicated cells (FIG. 14C). (FIG. 14D) UPK10 cells were treated with 10 mM cisplatin, 10 mM irinotecan or a combination for three days and released for three days. SA-β-gal positive cells were quantified using SPiDER SA-β-gal assay by flow cytometry. (FIG. 14E-FIG. 14F) UPK10 cells were treated with a combination of 10 mM cisplatin and 10 mM irinotecan for three days and released for three days. Senescent and non-senescent cells were sorted using gating strategies indicated in (FIG. 14E). Phase contrast images of sorted non-senescent and senescent UPK10 cells after replating were shown (FIG. 14F). (FIG. 14G) Sorted senescent and non-senescent cells from cisplatin and irinotecan treated UPK10 cells at the indicated time points post sorting (24 hours or 3 weeks) were labeled with BrdU for 24 hours and BrdU incorporation was examined by immunofluorescence staining and quantified. (FIG. 14H) 1×10⁶ sorted senescent and non-senescent cisplatin and irinotecan treated UPK10 cells (n=3 mice per group) were orthotopically transplanted into mouse bursa that covers mouse ovary. Shown are images of ovaries with tumor formed by non-senescent cells in one month and those without evidence of tumor formation by sorted senescent cells after two and half months. Data represent mean±SEM of three biologically independent experiments. Scale bar=100 mm in FIG. 14A and FIG. 14F, and =20 mm in FIG. 14G. P values were calculated using a two-tailed t-test.

FIG. 15A-FIG. 15D show TOP1 inhibitor irinotecan boosts SASP expression. (A-B) UPK10 cells were treated with 10 mM cisplatin, 10 mM irinotecan, a combination or 10 mM DMXAA for three days and released for three days. Expression of TOP1cc, TOP1, cyclin A, phosphor-p65, total p65, phosphor-p38 MAPK, total p38 MAPK, gH2AX, cGAS and a loading control β-actin was examined by immunoblot in the sorted non-senescent and senescent cells from the indicated treatment groups (FIG. 15A). Expression of the indicated SASP factors in sorted senescent and non-senescent UPK10 cells from the indicated treatment groups was determined by qRT-PCR (FIG. 15B). (n=3 biologically independent experiments). (FIG. 15C-FIG. 15D) Secretion of SASP factors under the indicated conditions was determined using an antibody array (FIG. 15C). Examples of changes in the secreted SASP factors were highlighted. The heatmap indicates the fold change (FC) in comparison with the control (Ctrl) UPK10 cells. Relative expression levels per replicate and average fold change differences are shown (FIG. 15D). Data represent mean±SEM. P values were calculated using a two-tailed t-test.

FIG. 16A-FIG. 16F show TOP1 inhibitor irinotecan boosts SASP through TOP1cc-regulated cGAS pathway. (FIG. 16A) Expression of TOP1 and a loading control β-actin in UPK10 cells expressing the indicated shTOP1s or a shControl was determined by immunoblot. (FIG. 16B) Expression of cGAS and a loading control β-actin in UPK10 cells expressing the indicated shcGASs or a shControl was determined by immunoblot. (FIG. 16C) Expression of TOP1cc in UPK10 cells expressing the indicated shTOP1s or a shControl was determined by slot blot. Expression of histone H3 was used as a control. (FIG. 16D) UPK10 cells were treated with 10 mM cisplatin, 10 mM irinotecan or a combination for three days and released for three days. Expression of the indicated SASP factors in the sorted nonsenescent and senescent cells was determined by qRT-PCR (n=3 biologically independent experiments). (FIG. 16E-FIG. 16F) Secretion of SASP factors under the indicated conditions was determined by an antibody array (FIG. 16E). Examples of changes in the secreted SASP factors were highlighted. The heatmap indicates the fold change (FC) in comparison with the control (Ctrl) or senescent UPK10 cells sorted from cisplatin and irinotecan combination treatment (Cisp+IRT). Relative expression levels per replicate and average fold change differences are shown (FIG. 16F). Data represent mean SEM of. P values were calculated using a two-tailed t-test.

FIG. 17A-FIG. 17H show adoptive transfer of SASP-boosted therapy-induced senescent cells sensitizes ovarian tumor to anti-PD-1 treatment. (FIG. 17A) Schematics of experimental design. GFP-expressing UPK10 cells were orthotopically transplanted into the mouse bursa for two weeks to allow for tumor formation. The indicated control or sorted senescent UPK10 cells ex vivo induced by cisplatin, irinotecan or a combination of cisplatin and irinotecan were i.p. injected on day 15 and 22 and followed with anti-PD-1 antibody treatment on day 16, 19, 23 and 26. In addition, transfer of DMAXX ex vivo treated UPK10 cells were included as a control. Note that sorted non-senescent cells were used as control cells. (FIG. 17B) At the end of two weeks of treatment, immunofluorescent staining revealed infiltration of injected non-senescent and senescent UPK10 cells (GFP and mCherry double positive) into the pre-established orthotopic tumors (only GFP positive). (FIG. 17C) Outline of experimental groups into which mice were randomized. Please note that control cells are sorted non-senescent cells. (D-E) At the end of two weeks of treatment, reproductive tracts with tumors from the indicated treatment groups were dissected (FIG. 17D) and tumor weights were measured as a surrogate for tumor burden (FIG. 17E). (n=5 biologically independent mice per group). (FIG. 17F) After stopping the treatment, the mice from the indicated groups were followed for survival. Shown are the Kaplan-Meier survival curves of mice from the indicated treatment groups (n=5 biologically independent mice per group). (FIG. 17G and FIG. 17H) Fold changes in percentage of CD69+/CD8+ T cells in CD8+ T cell population and CD11b+ dendritic cells in dendritic cell population (normalized by tumor weight) were determined in tumors dissected from the indicated treatment groups (n=5 biologically independent mice per group). Data represent mean±SEM. Scale bar=200 mm in FIG. 17B. β-values were calculated using two tailed t test in FIG. 17E, log-rank (Mantel-Cox) test in FIG. 17F, and multiple t test in FIG. 17G and FIG. 17H. n.s.: not significant.

FIG. 18A-FIG. 18K show isolation of SASP-boosted therapy-induced senescent cells, related to FIG. 14A-FIG. 14H. (FIG. 18A-FIG. 18C) UPK10 cells were treated with the indicated concentration of cisplatin (FIG. 18A) or irinotecan (FIG. 18B and FIG. 18C) for three days. After three days of release, expression of the indicated SASP factors was examined by qRT-PCR (FIG. 18A and FIG. 18B). Level of TOP Ice in the irinotecan-treated cells was examined by slot blot (FIG. 18C). (n=4 biologically independent experiments). (FIG. 18D) Percentage of dead cells in senescent UPK10 cells induced by a combination of 10 μM cisplatin and 10 μM irinotecan before and after flow cytometry sorting. (n=3 biologically independent experiments). (FIG. 18E and FIG. 18F) ID8 cells were treated with the indicated concentration of cisplatin (FIG. 18E) or irinotecan (FIG. 18F) for three days. After three days of release, expression of the indicated SASP factor was examined by qRT-PCR. (n=4 biologically independent experiments). (FIG. 18G-FIG. 18I) ID8cells were treated with 10 M cisplatin for three days and released for three days. SA-β-gal activity was examined (FIG. 18G) and quantified (FIG. 18H). Expression of the indicated proteins were examined by immunoblot in the indicated cells (FIG. 18I). (FIG. 18J) ID8cells were treated with 10 M cisplatin, 10 M irinotecan or a combination for three days and released for three days. SA-β-gal positive cells were quantified using SPiDER SA-β-gal assay by flow cytometry. (FIG. 18K) Sorted senescent and non-senescent cells from cisplatin and irinotecan treated ID8 cells at the indicated time points post sorting (24 hrs or 3 weeks) were labeled with BrdU for 24 hrs and BrdU incorporation was examined by immunofluorescence staining and quantified.(n=3 biologically independent experiments). Data represent mean±SEM. Scale bar=100 m in S1G and 20 m in S1K. P values were calculated using a two-tailed t-test.

FIG. 19A-FIG. 19D show TOP1 inhibitor irinotecan boosts SASP expression in cisplatin-induced ID8 senescent cells, related to FIG. 15A-FIG. 15D. (A-B)ID8cells were treated with 10 M cisplatin, 10 M irinotecan, a combination, or 10 μM DMXAA for three days and released for three days. Expression of TOP1cc, TOP1 and a loading control b-actin examined by immunoblot in the indicated cells (FIG. 19A). Expression of the indicated SASP factors in sorted senescent and non-senescent ID8cells from the indicated treatment was determined by qRT-PCR (n=3 biologically independent experiments) (FIG. 19B). (FIG. 19C and FIG. 19D) STING dimerization induced by DMXAA treatment was determined by immunoblot in UPK10 (FIG. 19C) and ID8 (FIG. 19D) cells. Data represent mean±SEM. P values were calculated using a two-tailed t-test.

FIG. 20A-FIG. 20D show irinotecan-boosted SASP in senescent ID8 cells depends on cGAS and TOP1, related to FIG. 16A-FIG. 16F. (FIG. 20A) Expression of TOP1 and a loading control b-actin in ID8cells expressing the indicated shTOP1sor a shControl was determined by immunoblot. (FIG. 20B) Expression of cGAS and a loading control b-actin in ID8cells expressing the indicated shcGASs or a shControl was determined by immunoblot. (FIG. 20C) Expression of TOP1cc in ID8cells expressing the indicated shTOP1sor a shControl was determined by slot blot. (FIG. 20D) ID8cells were treated with 10 M cisplatin, 10 M irinotecan or a combination for three days and released for three days. Expression of the indicated SASP factors in the sorted indicated non-senescent and senescent cells was determined by qRT-PCR (n=3 biologically independent experiments). Data represent mean±SEM. P values were calculated using a two-tailed t-test.

FIG. 21A-FIG. 21D show adoptive transfer of SASP-boosted senescent cells does not display overt toxicity, related to FIG. 17A-FIG. 17G. (FIG. 21A) Confirmation of GFP and mCherry expression in UPK10 cells used for generating pre-established tumors and adoptive transfer. GFP positive cells were used to generate orthotopic tumors, and GFP and mCherry double positive cells were used for senescence induction and subsequent transfer. (FIG. 21B) The gating strategy used in the present study. (FIG. 21C) Fold changes in percentage of CD69+/CD4+ T cells in CD4+ T cell population (normalized by tumor weight) were determined in tumors dissected from the indicated treatment groups (n=5 biologically independent mice per group). (FIG. 21C) Body weight of mice from the indicated treatment groups during the entire experimental period (n=5 biologically independent mice per group). Data represent mean±SEM. Scale bar=20 m in FIG. 21A. P-values were calculated using multiple t-test. n.s.: not significant

FIG. 22 shows the percentage of the indicated immune cells in tumor samples. The tumors with indicated treatment were digested, and the single cells from tumors were used for immune panel analysis.

### DETAILED DESCRIPTION OF THE INVENTION

As disclosed herein, methods and compositions are described which are useful in treatment of cancer in a mammalian subject. The inventors have shown that treatment of cancer cells with a chemotherapeutic agent and topoisomerase inhibitor induces senescence and SASP resulting in therapy-induced senescent cells. The TIS cells are then administered to the subject whereby the TIS cells home to any remaining cancer cells. The cytokines and/or chemokines released by the TIS cells “light up” the cancer cells, where, especially in conjunction with a checkpoint inhibitor, the immune system is able to locate and eradicate the cancer cells. This strategy is particularly effective in sensitizing the cancer cells to other therapies, such as checkpoint therapy.

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

“Patient” or “subject” as used herein means a mammalian animal, including a human, a veterinary or farm animal, a domestic animal or pet, and animals normally used for clinical research. In one embodiment, the subject of these methods and compositions is a human.

As used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing, inhibiting, or reducing the extent of metastasis, and/or extending the survival of an animal having cancer compared to control.

As used herein for the described methods and compositions, the term “antibody” refers to an intact immunoglobulin having two light and two heavy chains or fragments thereof capable of binding to a biomarker protein or a fragment of a biomarker protein. Thus, a single isolated antibody or an antigen-binding fragment thereof may be a monoclonal antibody, a synthetic antibody, a recombinant antibody, a chimeric antibody, a humanized antibody, a human antibody, or a bi-specific antibody or multi-specific construct that can bind two or more target biomarkers.

The term “antibody fragment” as used herein for the described methods and compositions refers to less than an intact antibody structure having antigen-binding ability. Such fragments, include, without limitation, an isolated single antibody chain or an scFv fragment, which is a recombinant molecule in which the variable regions of light and heavy immunoglobulin chains encoding antigen-binding domains are engineered into a single polypeptide. Other scFV constructs include diabodies, i.e., paired scFvs or non-covalent dimers of scFvs that bind to one another through complementary regions to form bivalent molecules. Still other scFV constructs include complementary scFvs produced as a single chain (tandem scFvs) or bispecific tandem scFvs.

Other antibody fragments include an Fv construct, a Fab construct, an Fc construct, a light chain or heavy chain variable or complementarity determining region (CDR) sequence, etc. Still other antibody fragments include monovalent or bivalent minibodies (miniaturized monoclonal antibodies) which are monoclonal antibodies from which the domains non-essential to function have been removed. In one embodiment, a minibody is composed of a single-chain molecule containing one VL, one VH antigen-binding domain, and one or two constant “effector” domains. These elements are connected by linker domains. In still another embodiment, the antibody fragments useful in the methods and compositions herein are “unibodies”, which are IgG4 molecules from with the hinge region has been removed.

The terms “analog”, “modification” and “derivative” refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined herein. Preferably, the analog, modification or derivative has at least the same desired activity as the native molecule, although not necessarily at the same level. The terms also encompass purposeful mutations that are made to the reference molecule. Particularly preferred modifications include substitutions that are conservative in nature, i.e., those substitutions that take place within a family of amino acids that are related in their side chains. Specifically, amino acids are generally divided into four families: acidic, basic, non-polar and uncharged polar. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-20 conservative or non-conservative amino acid substitutions, so long as the desired function of the molecule remains intact. One of skill in the art can readily determine regions of the molecule of interest that can tolerate change by reference to Hopp/Woods and Kyte Doolittle plots, well known in the art.

TOP1 inhibitors are clinically used for cancer therapy. As demonstrated herein, TOP inhibitors have additional applications to sensitize tumors to immunotherapy, especially targeting cancer cells that become resistant or senescent in response to therapies such as chemotherapy or radiotherapy. Here we show that topoisomerase 1-DNA covalent cleavage complex (TOP1cc) is both necessary and sufficient for cGAS-mediated cytoplasmic chromatin recognition and SASP during senescence. TOP Ice localizes to cytoplasmic chromatin and TOP1 interacts with cGAS to enhance the binding of cGAS to DNA. Retention of TOP1cc to cytoplasmic chromatin depends on its stabilization by the chromatin architecture protein HMGB2. Functionally, the HMGB2-TOP1cc-cGAS axis determines the response of orthotopically transplanted ex vivo therapy-induced senescent cells to immune checkpoint blockade in vivo. Together, these findings establish a HMGB2-TOP1cc-cGAS axis that enables cytoplasmic chromatin recognition and response to immune checkpoint blockade.

cGAS is essential for the antitumor effect of immune checkpoint blockades such as anti-PD-L1 antibody¹⁹. Here it is described that TOP Ice plays a critical role in mediating recognition of CCF (cytoplasmic chromatin fragments) by cGAS and the associated SASP during senescence. Mechanistically, HMGB2 stabilizes TOP1cc to enhance the binding of cGAS to dsDNA. Indeed, the HMGB2-TOP1cc-cGAS axis determines the response of orthotopically transplanted ex vivo therapy-induced senescent cells to immune checkpoint blockade in vivo. Also described herein is the use of TOP inhibitors in inducing senescence in tumor cells and using these cells to treat cancer.

### Methods

Provided herein, in one aspect, is a method of treating cancer in a subject in need thereof. The method includes administering therapy-induced senescent (TIS) cells and an immune checkpoint inhibitor to the subject. In one embodiment, the method includes sensitizing cancer cells to checkpoint therapy using TIS cells.

Cancer therapy has traditionally relied on cytotoxic treatment strategies on the assumption that complete cellular destruction of tumors optimizes the potential for patient survival. This view has limited the treatment options that oncologists have at their disposal to toxic compounds and high dose radiation. These approaches may produce complete cell death within a solid tumor and can cause severe side effects in patients. Such cancers often develop resistance to treatment and recur or progress to advanced primary and metastatic tumors. An alternative strategy is the induction of cytostasis, which permanently disables the proliferative capacity of cells without inducing cancer cell death. Initial clinical studies utilizing cytostatic treatments have yielded promising preliminary results, suggesting that these treatments may be as effective as cytotoxic therapies in preventing continued tumor growth. This approach to treatment could provide equivalent or prolonged survival with fewer and less severe side effects related to cytotoxicity and may provide a more realistic goal for the chronic management of some cancers. See, Ewald et al, Therapy-Induced Senescence in Cancer, J Natl Cancer Inst. 2010 Oct. 20; 102(20): 1536-1546, which is incorporated herein by reference.

A promising approach to induction of cytostasis in tumor cells is therapy-induced senescence. Senescent cells remain viable and metabolically active but are permanently growth arrested. In contrast to cells undergoing apoptosis or mitotic catastrophe in response to conventional cytotoxic drugs, senescent cells may persist indefinitely. As described herein, senescent cells can be exploited to “light up” remaining tumor cells, making those residual cells more susceptible to treatment and immune response.

Topoisomerase 1 (TOP1) is responsible for relaxing higher order topological DNA structures during DNA replication and gene transcription¹⁸. TOP1 forms a stable protein-DNA cleavage complex (TOP1cc) through its enzymatic activity and TOP1 becomes covalently bound to the catalytically generated DNA strand break¹⁸. Trapped or persistent TOP Ice induced by TOP1 inhibitors such as camptothecin are harmful to normal cellular function because they block both DNA and RNA polymerases¹⁸. However, the role of TOP1cc in senescence has never been explored.

In one embodiment, the method of treating cancer includes obtaining cancer cells from a subject; treating the cancer cells ex vivo with an effective amount of a chemotherapeutic agent, and an inhibitor of TOP1, TOP2, or both, to produce therapy induced senescent (TIS) cells; and administering the TIS cells to the same subject. In another embodiment, the cancer cells are treated ex vivo with an effective amount of an inhibitor of TOP1, TOP2, or both, to produce therapy induced senescent (TIS) cells.

In one embodiment, the TIS cells are derived from the subject who will be receiving the therapy. In one embodiment, the cells are removed from the subject prior to induction of senescence. The TIS cells may be derived from cancer cells from the subject, e.g., such as tumor cells from an excised or biopsied tumor, or blood cancer cells.

In one embodiment, the cells are collected from the patient. The cells may be pooled, concentrated, enriched or expanded to increase the number of cells available for treatment, using techniques known in the art, and described herein.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In certain embodiments, the cancer treated includes, but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma or Merkel cell carcinoma), and pancreas, as well as adenocarcinomas which include malignancies such as colon cancers, rectal cancer, renal-cell carcinoma, liver cancer, non-small cell lung cancer, cancer of the small intestine, cancer of the esophagus. The cancer may be at an early, intermediate, late stage or metastatic cancer.

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a skin cancer (e.g., a Merkel cell carcinoma or a melanoma (e.g., an advanced melanoma)), a kidney cancer (e.g., a renal cancer (e.g., a renal cell carcinoma (RCC) such as a metastatic RCC or clear cell renal cell carcinoma (CCRCC)), a liver cancer, a myeloma (e.g., a multiple myeloma), a prostate cancer (including advanced prostate cancer), a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), a brain cancer (e.g., a glioblastoma), an endometrial cancer, an anal cancer, a gastro-esophageal cancer, a thyroid cancer (e.g., anaplastic thyroid carcinoma), a cervical cancer, a neuroendocrine tumor (NET) (e.g., an atypical pulmonary carcinoid tumor), a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia). In one embodiment, the cancer is ovarian cancer. In another embodiment, the cancer is melanoma.

The cells that are derived from the subject to be treated are then treated ex vivo to induce senescence. In one embodiment, the cancer cells are treated ex vivo with a chemotherapeutic agent or radiation to induce senescence resulting in TIS cells. In one embodiment, the chemotherapeutic agent is a TOP1 or TOP2 inhibitor, or both. In another embodiment, the TOP1 or TOP2 inhibitor, or both, is administered in addition to another chemotherapeutic agent, or agents.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof. In one embodiment, chemotherapeutic agents include topoisomerase inhibitors. In one embodiment, the topoisomerase inhibitor is a topoisomerase 1 (also called TOP1) inhibitor. In another embodiment, the topoisomerase inhibitor is a topoisomerase 2 (also called TOP2) inhibitor. In one embodiment, the topoisomerase inhibitor is a pan-topoisomerase 1 (also called pan-TOP) inhibitor. In another embodiment, the chemotherapeutic agent is cisplatin.

Inhibitors of TOP1 include, but are not limited to irinotecan; irinotecan hydrochloride; camptothecin; topotecan; topotecan hydrochloride (Hycamtin, commercially available); indimitecan (LMP776) (Purdue); indotecan (LMP400) (Purdue); Genz-644282 (Genzyme); ONZEALD (etirinotecan pegol) (Nektar Therapeutics); gimatecan (Lee's Pharmaceutical Holdings, LLC); PEG-irinotecan (3SBio Inc.); belotecan hydrochloride (Chong Kun Dang Pharmaceutical Corp); IT-141 (Intezyne Inc); TBX.CE+irinotecan hydrochloride (irinotecan hydrochloride) (TheraBiologics Inc); PLX-038(ProLynx LLC); LMP-744 (Gibson Oncology LLC); Sinotecan (Jiangsu Chia-tai Tianqing Pharmaceutical Co Ltd); 2X-131 (Oncology Venture U.S. Inc); HSSYO-001 (Sichuan Sinovation Bio-technology Co Ltd); AR-67 (Vivacitas Oncology Inc); rubitecan (Vivacitas Oncology Inc); NK-012 (Nippon Kayaku Co Ltd); simmitecan hydrochloride (Shanghai Haihe Biopharma Co Ltd); ATT-1 IT (Aposense Ltd); BACPT-DP (DEKK-TEC Inc); APH-0201 (Aphios Corp); CZ-150729 (NMT Pharmaceuticals Pte Ltd); SNB-101 (SN BioScience); Small Molecule to Inhibit DNA Topoisomerase I for Lung Cancer (CAO Pharmaceuticals Inc); TRX-920 (TaiRx Inc); CBX-12 (Cybrexa Inc); moeixitecan (Jiangsu Chia-tai Tianqing Pharmaceutical Co Ltd), BAX 2398; BAX-2398; BAX2398; irinotecan hydrochloride nanoliposomal; irinotecan nydrochloride; Irinotecan liposome injection; irinotecan sucrosofate liposomal; irinotecan; liposomal irinotecan sucrosulfate; MM 398; MM-398; MM398; nal-IRI; nanoliposomal irinotecan hydrochloride; nanoliposomal irinotecan; onivyde; PEP 02; PEP-02; PEP02; SHP 673; SHP-673; SHP673; PEG-irinotecan; CZ-48; IT-141; NLG-207; PEN-866; BO-1978; LMP-135; PCS-11T (SN-38 prodrug); ZBH-01; DFP 13318; DFP-13318; DFP13318; PEG-SN-38 conjugate; Pegylated form of SN38; PEGylated-SN-38 conjugate; PL 0264; PL-0264; PL0264; PLX 0264; PLX 038; PLX-0264; PLX0264; PLX038; and Ultra-long acting PEG-SN-38. See also, e.g., Burton et al. Clin Cancer Res. 2018 Dec. 1; 24(23):5830-5840, which is incorporated herein by reference.

Inhibitors of TOP2 include, without limitation, teniposide, daunorubicin, aurintricarboxylic acid, HU-331, etoposide, doxorubicin, mitoxantrone, dexrazoxane, aclarubicin, amsacrine, and ellipticine. Other suitable TOP2 inhibitors include, without limitation, iodoquinol+metronidazole, iodoquinol+sulfaguanidine, amsacrine, fleroxacin, idarubicin hydrochloride, teniposide, 2X-111 (Glutathione-enhanced, PEGylated Liposomal Doxorubicin, previously named 2B3-101) (Allarity Therapeutics A/S), Aldoxorubicin (ImmunityBio Inc), Annamycin (Moleculin Biotech Inc), Idronoxil (NXP-001) (Noxopharm Ltd), epirubicin, camsirubicin (MNPR-201; GPX-150; 5-imino-13-deoxydoxorubicin; analog of doxorubicin) (Monopar Therapeutics Inc), pegylated doxorubicin (GP Pharm SA), pirarubicin, pixantrone dimaleate (Servier Laboratories Ltd), Razoxane (TRP-1001) (Tryp Therapeutics Inc), SQ-3370 (Shasgi Inc), and Vosaroxin (DB106) (Denovo Biopharma LLC). See also, e.g., Saleh et al, Reversibility of chemotherapy-induced senescence is independent of autophagy and a potential model for tumor dormancy and cancer recurrence, BioRxiv, doi: 10.1101/099812, posted Jan. 11, 2017. This document is incorporated herein by reference.

In one embodiment, the cancer cells are treated with the chemotherapeutic agent prior to treatment with the TOP inhibitor. In another embodiment, the cancer cells are treated with the chemotherapeutic agent after treatment with the TOP inhibitor. In yet another embodiment, the cancer cells are treated with the chemotherapeutic agent essentially simultaneously with treatment with the TOP inhibitor, or the treatment periods overlap.

In one embodiment, the cancer cells are treated with more than one chemotherapeutic agent, optionally in conjunction with radiation. The cells are treated with an effective amount of the chemotherapeutic agent or agents, and/or radiation, suitable to produce senescence in the cancer cells, resulting in TIS cells. In one embodiment, the cells are treated ex vivo with a combination of an inhibitor of TOP1, TOP2, or both and cisplatin. In one embodiment, the TOP1 inhibitor is irinotecan. In one embodiment, the TOP1 inhibitor is camptothecin. In one embodiment, the TOP2 inhibitor is etoposide.

The cells are treated with an effective amount of the chemotherapeutic agent and the inhibitor of TOP1, TOP2, or both. It should be understood that the “effective amount” for the chemotherapeutic agent or agents or TOP inhibitor(s) may vary depending upon the agent(s) selected for use in the method, and may be determined by the person of skill in the art. In one embodiment an effective amount for the chemotherapeutic agent or TOP inhibitor includes without limitation about 0.1 μM to about 100 μM. In one embodiment, the range of effective amount is 0.001 to 0.01 μM. In another embodiment, the range of effective amount is 0.001 to 0.1 μM. In another embodiment, the range of effective amount is 0.001 to 1 μM. In another embodiment, the range of effective amount is 0.001 to 10 μM. In another embodiment, the range of effective amount is 0.001 to 20 μM. In another embodiment, the range of effective amount is 0.01 to 25 μM. In another embodiment, the range of effective amount is 0.01 to 0.1 μM. In another embodiment, the range of effective amount is 0.01 to 1 μM. In another embodiment, the range of effective amount is 0.01 to 10 μM. In another embodiment, the range of effective amount is 0.01 to 20 μM. In another embodiment, the range of effective amount is 0.1 to 25 μM. In another embodiment, the range of effective amount is 0.1 to 1 μM. In another embodiment, the range of effective amount is 0.1 to 10 μM. In another embodiment, the range of effective amount is 0.1 to 20 μM. In another embodiment, the range of effective amount is 1 to μM. In another embodiment, the range of effective amount is 1 to μM. In another embodiment, the range of effective amount is 1 to 10 μM. In another embodiment, the range of effective amount is 1 to 20 μM. In another embodiment, the range of effective amount is 5 to 15 μM. In another embodiment, the range of effective amount is about 10 μM. Still other doses falling within these ranges are expected to be useful. The effective amount of the chemotherapeutic agent(s) and TOP inhibitor(s) may be individually chosen based on the agents selected and other factors, e.g., number of cells being treated, type of cancer, etc.

In one embodiment, the cells which have been removed from the subject are contacted with a chemotherapeutic agent for a time sufficient to induce senescence. The time may range from minutes to days or weeks. In one embodiment, the cells are contacted with the chemotherapeutic agent for about 5 minutes to about 4 weeks. In another embodiment, the cells are contacted with the chemotherapeutic agent for about 1 hour to about 2 weeks. In one embodiment, the cells are contacted with the chemotherapeutic agent for about 1, 2, 3, 4, 5, 6, 7, 8, 9, 10, 11, 12, 13, 14, 15, 16, 17, 18, 19, 20, 21, 22, 23, or 24 hours. In another embodiment, the cells are contacted with the chemotherapeutic agent for about 1, 2, 3, 4, 5, 6, 7, 8, 9, 10, 11, 12, 13, 14, 15, 16, 17, 18, 19, 20, 21, 22, 23, 24, 25, 26, 27 or 28 days. The chemotherapeutic agent may be administered more than once in the treatment period.

In one embodiment, the cells which have been removed from the subject are contacted with a TOP1 inhibitor, a TOP2 inhibitor, or both for a time sufficient to induce the release of cytokines, chemokines, and/or other factors associated with senescence. The time may range from minutes to days or weeks. In one embodiment, the cells are contacted with the TOP1 inhibitor, a TOP2 inhibitor, or both for about 5 minutes to about 4 weeks. In another embodiment, the cells are contacted with the TOP1 inhibitor, a TOP2 inhibitor, or both for about 1 hour to about 2 weeks. In one embodiment, the cells are contacted with the TOP1 inhibitor, a TOP2 inhibitor, or both for about 1, 2, 3, 4, 5, 6, 7, 8, 9, 10, 11, 12, 13, 14, 15, 16, 17, 18, 19, 20, 21, 22, 23, or 24 hours. In another embodiment, the cells are contacted with the TOP1 inhibitor, a TOP2 inhibitor, or both for about 1, 2, 3, 4, 5, 6, 7, 8, 9, 10, 11, 12, 13, 14, 15, 16, 17, 18, 19, 20, 21, 22, 23, 24, 25, 26, 27 or 28 days. The TOP inhibitor may be administered more than once in the treatment period.

In one embodiment, the methods described herein include identifying whether the ex vivo treated cells have obtained senescence. In one embodiment, cells are sorted into senescent and non-senescent cells. Such techniques include sorting by size and/or granularity (Meng et al, Radiation-inducible Immunotherapy for Cancer: Senescent Tumor Cells as a Cancer Vaccine Molecular Therapy, 20(5):1046-1055, May 2012) (larger size than senescent cells), cyclin A expression (negative for senescent cells), SASP expression (increased for senescent cells), and/or TOP1cc level (increased for senescent cells).

In one embodiment, that involves detection of a senescence-associated secretory phenotype (SASP) in the treated cells. The SASP includes several families of soluble and insoluble factors including, those in the table below. Detection of the SASP may include detection of one or more of the following factors: TECK, ENA-78, I-309, I-TAC, GM-CSE, G-CSE, IFN-γ, BLC, MIF, amphiregulin, epiregulin, heregulin, EGF, bFGF, HGF, KGF (FGF7), VEGF, angiogenin, SCF, SDF-1, PIGF, NGF, IGFBP-2, IGFBP-3, IGFBP-4, IGFBP-6, IGFBP-7, MMP-1, MMP-3, MMP-10, MMP-12, MMP-13, MMP-14, TIMP-1, TIMP-2, PAI-1, PAI-2, tPA, uPA, cathepsin B, ICAM-1, ICAM-3, OPG, sTNFRI, TRAIL-R2, Fas, and sTNFRII. See, e.g., Coppe et al, The Senescence-Associated Secretory Phenotype: The Dark Side of Tumor Suppression, Annu Rev Pathol. 2010; 5: 99-118, which is incorporated by reference herein.

In another embodiment, the level of TOPcc is detected in the cells wherein an increase in TOPcc levels is indicative of senescence in the cells. In yet another embodiment, the treated cancer cells are assayed for SA-β-Gal to detect senescence. It is contemplated that in some of the methods, a portion of the treated cells are used as a measurement to determine whether senescence has been obtained. In some embodiments, these cells may be stained and/or fixed, and thus, not suitable for readministration into the subject. In other embodiments, the cells are stained and/or sorted, and administered to the subject.

The TIS cells or a pharmaceutical composition containing the TIS cells are then administered to the subject. The cells may be administered using any suitable route of administration. For example, compositions may be administered via intravenous, parenteral, subcutaneous, intramuscular, intracranial, intraorbital, ophthalmic, intraventricular, intracapsular, intraspinal, intracisteral, intraperitoneal, intranasal, or aerosol administration. The route of administration may be determined by the person of skill based on various factors including without limitation the type of cancer being treated. In one embodiment, the TIS cells are injected in the area where the cancer is/was located. In another embodiment, the TIS cells are administered intravenously.

In one embodiment, the effective amount of the TIS cells ranges from about 1 cell to about 100,000,000 cells, including all integers or fractional amounts within the range. In one embodiment, the effective amount of the TIS cells ranges from about 10,000 cells to about 10,000,000 cells, including all integers or fractional amounts within the range. In one embodiment, the effective amount of the TIS cells ranges from about 100,000 cells to about 5,000,000 cells, including all integers or fractional amounts within the range. Other ranges and dosages may be determined by the person of skill taking into account various factors including, without limitation, the type of cancer, the size of the subject, etc.

In one embodiment, the subject is administered a checkpoint inhibitor in addition to the TIS cells. Immune checkpoints represent significant barriers to activation of functional cellular immunity in cancer, and antagonistic antibodies specific for inhibitory ligands on T cells including CTLA4 and programmed death-1 (PD-1) are examples of targeted agents being evaluated in the clinics. In one embodiment, the subject has previously received checkpoint therapy, prior to receiving TIS cell therapy. The subject may, in some embodiments, receive the same or different checkpoint therapy after administration of the TIS cells.

Immune checkpoint molecules that may be targeted for blocking or inhibition include, but are not limited to, CTLA-4, 4-1BB (CD137), 4-1BBL (CD137L), PDL1, PDL2, PD1, CD134, B7-H3, B7-H4, BTLA, HVEM, TIM3, GAL9, LAG3, TIM3, B7H3, B7H4, VISTA, KIR, 2B4, CD160 (also referred to as BY55) and CGEN-15049. In one embodiment, the checkpoint inhibitor is PD-1 inhibitor. In another embodiment, the checkpoint inhibitor is PD-L1 inhibitor.

Immune checkpoint inhibitors include antibodies, or antigen binding fragments thereof, or other binding proteins, that bind to and block or inhibit the activity of one or more of CTLA-4, PDL1, PDL2, PD1, CD134, B7-H3, B7-H4, BTLA, HVEM, TIM3, GAL9, LAG3, TIM3, B7H3, B7H4, VISTA, KIR, 2B4, CD160 and CGEN-15049. Suitable immune checkpoint inhibitors include those that block PD-1, such as pembrolizumab, nivolumab, AGEN 2034, BGB-A317, BI-754091, CBT-501 (genolimzumab), MEDI0680, MGA012, PDR001, PF-06801591, REGN2810 (SAR439684), and TSR-042. MK-3475 (PD-1 blocker) Nivolumab, CT-011 Immune checkpoint inhibitors also include those that block PD-L1, such as durvalumab, atezolizumab, avelumab, and CX-072. Other suitable inhibitors include Anti-B7-H1 (MEDI4736), AMP224, BMS-936559, MPLDL3280A, and MSB0010718C.

Suitable immune checkpoint inhibitors include those that block CTLA-4, such as AGEN 1884, ipilimumab, and tremelimumab.

In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-LAG-3 antibody selected from the group consisting of: BMS-986016 and LAG525. In some embodiments, the additional therapeutic agent is an anti-OX-40 antibody selected from: MEDI6469, MEDI0562, and MOXR0916. In some embodiments, the additional therapeutic agent is the anti-4-1BB antibody PF-05082566. The present disclosure provides compositions and methods that include blockade of immune checkpoints. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In addition, more than one immune checkpoint inhibitor (e.g., anti-PD-1 antibody and anti-CTLA-4 antibody) may be used in combination with the TIS cells.

The subject is treated with an effective amount of the checkpoint inhibitor. It should be understood that the “effective amount” for the checkpoint inhibitor may vary depending upon the agent(s) selected for use in the method, and may be determined by the person of skill in the art. In one embodiment an effective amount for the checkpoint inhibitor includes without limitation about 1 μg to about 25 mg. In one embodiment, the range of effective amount is 0.001 to 0.01 mg. In another embodiment, the range of effective amount is 0.001 to 0.1 mg. In another embodiment, the range of effective amount is 0.001 to 1 mg. In another embodiment, the range of effective amount is 0.001 to 10 mg. In another embodiment, the range of effective amount is 0.001 to 20 mg. In another embodiment, the range of effective amount is 0.01 to 25 mg. In another embodiment, the range of effective amount is 0.01 to 0.1 mg. In another embodiment, the range of effective amount is 0.01 to 1 mg. In another embodiment, the range of effective amount is 0.01 to 10 mg. In another embodiment, the range of effective amount is 0.01 to 20 mg. In another embodiment, the range of effective amount is 0.1 to 25 mg. In another embodiment, the range of effective amount is 0.1 to 1 mg. In another embodiment, the range of effective amount is 0.1 to 10 mg. In another embodiment, the range of effective amount is 0.1 to 20 mg. In another embodiment, the range of effective amount is 1 to 25 mg. In another embodiment, the range of effective amount is 1 to 5 mg. In another embodiment, the range of effective amount is 1 to 10 mg. In another embodiment, the range of effective amount is 1 to 20 mg. Still other doses falling within these ranges are expected to be useful. The effective amount of the checkpoint inhibitor may be individually chosen based on the agent selected and other factors, e.g., size of the patient, type of cancer, etc.

In one embodiment, the TIS cells and checkpoint inhibitor are administered approximately simultaneously. In another embodiment, the TIS cells are administered prior to checkpoint inhibitor. In another embodiment, the TIS cells are administered subsequent to the checkpoint inhibitor.

In another embodiment, the method includes administering a chemotherapeutic agent to the subject in addition to the TIS cells, and optionally with a checkpoint inhibitor.

The chemotherapeutic agents, TIS cells and checkpoint inhibitors may be administered using any suitable route of administration. For example, compositions may be administered via intravenous, parenteral, subcutaneous, intramuscular, intracranial, intraorbital, ophthalmic, intraventricular, intracapsular, intraspinal, intracisteral, intraperitoneal, intranasal, or aerosol administration. The route of administration for each composition (e.g., TIS cells, chemotherapeutic agents, checkpoint inhibitors) may be determined individually and may be the same or different.

The compositions described herein (e.g., TIS cells, chemotherapeutic agents, checkpoint inhibitors) are administered in an amount that is sufficient to treat or prevent the disease or disorder, or to treat the symptoms of the disease or disorder, in a subject. The combination of substances (or compounds) is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

Other therapeutic benefits or beneficial effects provided by the methods described herein may be objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis.

A therapeutic benefit or improvement therefore may be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass (in terms of size or cell numbers) by inhibiting progression or worsening of the tumor or cancer, can reduce mortality and prolong lifespan even if only for a few days, weeks or months, even though a portion or the bulk of the tumor or cancer mass remains.

A reduction or inhibition of cancer can be measured relative to the incidence observed in the absence of the treatment and, in further testing, inhibits tumor growth. The tumor inhibition can be quantified using any convenient method of measurement. Tumor growth can be reduced by about 10%, 20%, 30%, 40%, 50%, 60%, 70%, 80%, 90%, 95% or greater.

### Compositions

In another aspect, a pharmaceutical composition comprising the TIS cells as described herein, is provided. In one embodiment, the pharmaceutical composition includes a pharmaceutically acceptable carrier, excipient, adjuvant, or diluent. The pharmaceutical composition may further comprise an immune checkpoint inhibitor or chemotherapeutic agent, as described herein. Any reference herein to administration of TIS cells also refers, in one embodiment, to administration of a pharmaceutical composition comprising TIS cells.

In yet another aspect, a pharmaceutical composition produced by the following method is provided. The method includes obtaining cancer cells from a subject; and treating the cancer cells ex vivo with an inhibitor of TOP1, TOP2, or both to produce therapy induced senescent (TIS) cells, as described more fully herein. In one embodiment, the method includes obtaining cancer cells from a subject; and treating the cancer cells ex vivo with a chemotherapeutic agent and an inhibitor of TOP1, TOP2, or both to produce therapy induced senescent (TIS) cells, as described more fully herein. The method includes optionally sorting the cells for senescence or confirming senescence. In one embodiment, the cells are sorted for senescence by size and/or granularity. See, e.g., Meng et al, Radiation-inducible Immunotherapy for Cancer: Senescent Tumor Cells as a Cancer Vaccine Molecular Therapy, 20(5):1046-1055, May 2012, which is incorporated herein by reference.

In one embodiment, the cells are collected from the patient. The cells may be pooled, concentrated, enriched or expanded to increase the number of cells available for treatment, using techniques known in the art, and described herein.

By “pharmaceutically acceptable carrier, excipient, or diluent” is meant a solid and/or liquid carrier, in in dry or liquid form and pharmaceutically acceptable. The compositions are typically sterile solutions or suspensions. Examples of excipients which may be combined with the TIS cells include, without limitation, solid carriers, liquid carriers, adjuvants, amino acids (glycine, glutamine, asparagine, arginine, lysine), antioxidants (ascorbic acid, sodium sulfite or sodium hydrogen-sulfite), binders (gum tragacanth, acacia, starch, gelatin, polyglycolic acid, polylactic acid, poly-d,l-lactide/glycolide, polyoxaethylene, polyoxapropylene, polyacrylamides, polymaleic acid, polymaleic esters, polymaleic amides, polyacrylic acid, polyacrylic esters, polyvinylalcohols, polyvinylesters, polyvinylethers, polyvinylimidazole, polyvinylpyrrolidon, or chitosan), buffers (borate, bicarbonate, Tris-HCl, citrates, phosphates or other organic acids), bulking agents (mannitol or glycine), carbohydrates (such as glucose, mannose, or dextrins), clarifiers, coatings (gelatin, wax, shellac, sugar or other biological degradable polymers), coloring agents, complexing agents (caffeine, polyvinylpyrrolidone, β-cyclodextrin or hydroxypropyl-β-cyclodextrin), compression aids, diluents, disintegrants, dyes, emulsifiers, emollients, encapsulating materials, fillers, flavoring agents (peppermint or oil of wintergreen or fruit flavor), glidants, granulating agents, lubricants, metal chelators (ethylenediamine tetraacetic acid (EDTA)), osmo-regulators, pH adjustors, preservatives (benzalkonium chloride, benzoic acid, salicylic acid, thimerosal, phenethyl alcohol, methylparaben, propylparaben, chlorhexidine, sorbic acid, hydrogen peroxide, chlorobutanol, phenol or thimerosal), solubilizers, sorbents, stabilizers, sterilizer, suspending agent, sweeteners (mannitol, sorbitol, sucrose, glucose, mannose, dextrins, lactose or aspartame), surfactants, syrup, thickening agents, tonicity enhancing agents (sodium or potassium chloride) or viscosity regulators. See, the excipients in “Handbook of Pharmaceutical Excipients”, 5^{th} Edition, Eds.: Rowe, Sheskey, and Owen, APhA Publications (Washington, D.C.), 2005 and U.S. Pat. No. 7,078,053, which are incorporated herein by reference. The selection of the particular excipient is dependent on the nature of the compound selected and the particular form of administration desired.

Solid carriers include, without limitation, starch, lactose, dicalcium phosphate, microcrystalline cellulose, sucrose and kaolin, calcium carbonate, sodium carbonate, bicarbonate, lactose, calcium phosphate, gelatin, magnesium stearate, stearic acid, or talc. Fluid carriers without limitation, water, e.g., sterile water, Ringer's solution, isotonic sodium chloride solution, neutral buffered saline, saline mixed with serum albumin, organic solvents (such as ethanol, glycerol, propylene glycol, liquid polyethylene glycol, dimethylsulfoxide (DMSO)), oils (vegetable oils such as fractionated coconut oil, arachis oil, corn oil, peanut oil, and sesame oil; oily esters such as ethyl oleate and isopropyl myristate; and any bland fixed oil including synthetic mono- or diglycerides), fats, fatty acids (include, without limitation, oleic acid find use in the preparation of injectables), cellulose derivatives such as sodium carboxymethyl cellulose, and/or surfactants.

Pharmaceutical compositions may be formulated for any appropriate route of administration. For example, compositions may be formulated for intravenous, parenteral, subcutaneous, intramuscular, intracranial, intraorbital, ophthalmic, intraventricular, intracapsular, intraspinal, intracisteral, intraperitoneal, intranasal, or aerosol administration. In some embodiments, pharmaceutical compositions are formulated for direct delivery to the tumor (intratumoral) or to the tumor environment. In another embodiment, pharmaceutical compositions are formulated for delivery to the lymph nodes.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc. Methods and agents well known in the art for making formulations are described, for example, in “Remington's Pharmaceutical Sciences,” Mack Publishing Company, Easton, Pa. Formulations may, for example, contain excipients, diluents such as sterile water, or saline, polyalkylene glycols such as polyethylene glycol, oils of vegetable origin, or hydrogenated napthalenes.

In one embodiment, the effective amount of the TIS cells ranges from about 1 cell to about 100,000,000 cells, including all integers or fractional amounts within the range. In one embodiment, the effective amount of the TIS cells ranges from about 10,000 cells to about 10,000,000 cells, including all integers or fractional amounts within the range. In one embodiment, the effective amount of the TIS cells ranges from about 100,000 cells to about 5,000,000 cells, including all integers or fractional amounts within the range. Other ranges and dosages may be determined by the person of skill taking into account various factors including, without limitation, the type of cancer, the size of the subject, etc.

In one embodiment, the above amounts represent a single dose. In another embodiment, the above amounts define an amount delivered to the subject per day. In another embodiment, the above amounts define an amount delivered to the subject per day in multiple doses. In still other embodiments, these amounts represent the amount delivered to the subject over more than a single day.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

Cyclic cGMP-AMP synthase (cGAS) is a pattern recognition cytosolic DNA sensor that is essential for cellular senescence. cGAS promotes inflammatory senescence-associated secretory phenotype (SASP) through recognizing cytoplasmic chromatin during senescence. cGAS-mediated inflammation is essential for the antitumor effects of immune checkpoint blockade. However, the mechanism by which cGAS recognizes cytoplasmic chromatin is unknown. Here we show that topoisomerase 1-DNA covalent cleavage complex (TOP Ice) is both necessary and sufficient for cGAS-mediated cytoplasmic chromatin recognition and SASP during senescence. TOP1cc localizes to cytoplasmic chromatin and TOP1 interacts with cGAS to enhance the binding of cGAS to DNA. Retention of TOP1cc to cytoplasmic chromatin depends on its stabilization by the chromatin architecture protein HMGB2. Functionally, the HMGB2-TOP1cc-cGAS axis determines the response of orthotopically transplanted ex vivo therapy-induced senescent cells to immune checkpoint blockade in vivo. Together, these findings establish a HMGB2-TOP1cc-cGAS axis that enables cytoplasmic chromatin recognition and response to immune checkpoint blockade.

### Example 1: Materials and Methods

### Cells and Culture Conditions

IMR90 human diploid fibroblasts were cultured according to American Type Culture Collection (ATCC) under low oxygen tension (2%) in DMEM (4.5 g per liter glucose) supplemented with 10% fetal bovine serum (FBS), L-glutamine (Thermo Fisher. Cat. No: 25030081), sodium pyruvate (Thermo Fisher. Cat. No: 11360070), nonessential amino acid (Thermo Fisher. Cat. No: 11140-050), sodium bicarbonate (Gibco. Cat. No: 25080094) and 1% penicillin/streptomycin (Corning. Cat. No: 30-002-CI). All experiments were performed on IMR90 fibroblasts cultured between the population doubling of 25 and 35. Human ovarian cancer cell line OVCAR3 obtained from ATCC and mouse ovarian cancer cell line ID8-Defb29 Vegf-a gifted by Dr. Jose R. Conejo-Garcia were cultured in RPMI 1640 supplemented with 10% FBS and 1% penicillin/streptomycin. The mouse ovarian cancer cell lines UPK10 and ID8 were cultured in RPMI 1640 supplemented with 10% fetal bovine serum (FBS) and 1% penicillin/streptomycin. These cell lines are authenticated at The Wistar Institute's Genomics Facility using short tandem repeat DNA profiling. Regular mycoplasma testing was performed using the LookOut Mycoplasma polymerase chain reaction (PCR) detection (Sigma, Cat. No: MP0035).

### Reagents, Plasmids and Antibodies

Etoposide was purchased from Sigma (Cat. No: E1383). Cisplatin was purchased from Selleck (Cat. No: S1166). Doxycycline was purchased from Selleck (Cat. No: S4163). Camptothecin was purchased from Selleck (Cat. No: S1288). 4′ 6-Diamidino-2-phenylindole dihydrochloride (DAPI) was purchased from Sigma (Cat. No: D9542). Cytochalasin B was purchased from Sigma (Cat. No: C6762). Spermidine was purchased from Sigma (Cat. No: S2626). Formaldehyde solution was purchased from Sigma (Cat. No: F8775). Paraformaldehyde (PFA) was purchased from Sigma (Cat. No: 158127). The DNA ladder was purchased from ThermoFisher (Cat. No: SM1333). Benzonase was purchased from Sigma (Cat. No: E1014).

The pMSCVpuro-eGFP-hcGAS, pBABE-puro-H-RAS^{G12V}, pBABE-puro-Empty and pGEX6P1-GST-cGAS plasmids were obtained from Addgene. pLKO.1-shHMGB2 (shHMGB2 #1: TRCN000000150009; shHMGB2 #2: TRCN0000019011) and pLKO.1-shTOP1 (TRCN0000059090) were obtained from the Molecular Screening Facility at the Wistar Institute. pLKO.1-shcGAS short hairpins were purchased from Sigma (TRCN0000146282, TRCN0000149984).pLentiCRISPR-HMGB2 was constructed by inserting the HMGB2 guide RNA (gRNA; 5′-AACACCCTGGCCTATCCA TT-3′(SEQ ID NO: 1)) as we previously published¹⁵. Tet-pLKO-puro-shHMGB2 was constructed using the Tet-pLKO-puro backbone (Addgene. Cat. No: 21915) and shHMGB2 sequence (forward: 5′-CCGGGCTCAACATTAGCTTCAGTATCTCGAGATAC TGAAGCTAATGTTGAGCTTTTTG-3′ (SEQ ID NO: 2); reverse: 5′-AATTCAAAAAGCTCAACATTAGCTTCAGTATCTCGA GATACTGAAGCTAATGTTGAGC-3′(SEQ ID NO: 3)).

Recombinant cGAS protein was purchased from Cayman (Cat. No: 22810). Recombinant HMGB2 protein was purchased from Prospec (Cat. No: PRO-888). Recombinant TOP1 protein was purchased from Prospec (Cat. No: ENZ-306). Recombinant TOP1 Y723F mutant protein was purchased from Speed Biosystems (Cat. No: OP10402). Recombinant his-tagged TOP1 protein was purchased from Sino Biological (Cat. No: 17455-H07B). ATP Solution (100 mM) was purchased from Thermo Fisher (Cat. No: R0441). GTP Solution (100 mM) was purchased from Thermo Fisher (Cat. No: R0461). SYBR™ Green I Nucleic Acid Gel Stain was purchased from Thermo Fisher (Cat. No: S7563). (ISD)₂ interferon stimulatory double strand DNA (dsDNA) was purchased from InvivoGen (Cat. No: tlrl-isdn).

The following antibodies were purchased from the indicated suppliers and used for immunoblotting or immunostaining at the indicated concentrations: mouse monoclonal anti-γH2AX (clone JBW301) (Millipore. Cat. No: 05-636), 1:500 for immunofluorescence; rabbit monoclonal anti-γH2AX (20E3) (Cell Signaling Technology. Cat. No: 9718), 1:500 for immunofluorescence; Alexa Fluor® 594 anti-γH2AX (2F3) (Biolegend. Cat. No: 613410), 1:200 for immunofluorescence; rabbit polyclonal anti-HMGB2 (Abcam. Cat. No: 67282), 1:1000 for immunoblotting and 1:500 for immunofluorescence; mouse monoclonal anti-cGAS (D9) (Santa Cruz. Cat. No: sc-515777), 1:200 for immunofluorescence, 1:1000 for immunoblotting; rabbit monoclonal anti-cGAS (D1D3G) (Cell Signaling Technology. Cat. No: 15102), 1:200 for immunofluorescence, 1:1000 for immunoblotting; rabbit monoclonal anti-STING (D2P2F) (Cell Signaling Technology. Cat. No: 13647S), 1:1000 for immunoblotting, rabbit polyclonal anti-Cyclin A (H432) (Santa Cruz. Cat. No: sc-751), 1:1000 for immunoblotting; mouse monoclonal anti-RAS (BD Biosciences. Cat. No: 610001), 1:1000 for immunoblotting; mouse monoclonal anti-P16 (JC8) (Santa Cruz. Cat. No: sc-56330), 1:1000 for immunoblotting; mouse monoclonal anti-P21 (187) (Santa Cruz. Cat. No: sc-817), 1:1000 for immunoblotting; mouse monoclonal anti-β-actin (Sigma. Cat. No: A2228), 1:10000 for immunoblotting; rabbit polyclonal anti-TOP1 (Proteintech. Cat. No: 20705-1-AP), 1:1000 for immunoblotting and 1:200 for immunofluorescence; mouse monoclonal anti-Topoisomerase I-DNA Covalent Complexes (TOP1cc) (clone 1.1A) (Millipore. Cat. No: MABE1084), 1:1000 for slot blot and 1:200 for immunofluorescence;

For flow cytometric analysis, APC/CY7 anti-CD69 (Cat. No: 104525), APC anti-CD4 (Cat. No: 100516), PE anti-CD8 (Cat. No: 100708), FITC anti-Granzyme B (Cat. No: 372206), PE/Cy7 anti-interferon-gamma (Cat. No: 505825) antibodies were purchased from Biolegend and used at 1:150 dilutions. Zombie yellow dye (Biolegend. Cat. No: 423103, 1:200) was used as a viability staining.

### Retrovirus and Lentivirus Infection

Retrovirus production and transduction were performed using Phoenix cells to package the infection viruses (Dr. Gary Nolan, Stanford University) (Nacarelli et al., 2019). Lentivirus was produced using the ViraPower kit (Invitrogen) based on manufacturer's instructions in the 293FT human embryonal kidney cell line by Lipofectamine 2000 transfection (Thermo Fisher. Cat. No: 11668019). Lentivirus was harvested and filtered with 0.45 μm filter 48 hours post transfection. Cells infected with lentiviruses were selected in 1 μg/ml puromycin 48 hours post infection.

### Senescence Induction and SA-β-Gal Staining

For oncogene-induced senescence, IMR90 cells were infected with retrovirus produced by pBABE-puro-H-RAS^{G12V} (Addgene) at 37° C. for 24 hr. A second round of infection was performed on the same target cells. Infected cells were drug-selected using 3 μg/ml puromycin (Nacarelli et al., 2019). For Etoposide-induced senescence, IMR90 or OVCAR3 cells at approximately 60-70% confluency were treated with 50 μM or 2 μM Etoposide for 48 hours. The treated cells were cultured in fresh medium and harvested at day 8. For Cisplatin-induced senescence, OVCAR3 or ID8-Defb29/Vegf-a cells at approximately 60-70% confluency were treated with 2 μM Cisplatin for 48 hours. The treated cells were cultured in fresh medium and harvested at day 8.

SA-β-Gal staining was performed as previously described (Nacarelli et al., 2019). Briefly, cells were fixed for 5 mins at room temperature in 2% formaldehyde/0.2 glutaraldehyde in PBS. After washing twice with PBS, cells were stained at 37° C. overnight in a non-CO₂ incubator in staining solution (40 mM Na₂HPO₄, pH 6.0, 150 mM NaCl, 2 mM MgCl₂, 5 mM K₃Fe(CN)₆, 5 mM K₄Fe(CN)₆, and 1 mg/ml X-gal. After counterstaining with Nuclear Fast Red solution (Ricca, Cat. No: R5463200500), slides were subjected to an alcohol dehydration series and mounted with Permount (FisherScientific. Cat. No: SP15-100). Slides were examined using a Zeiss AxioImager A2.

UPK10 and ID8 cells were treated with 10 M Cisplatin, 10 M Irinotecan, or a combination for three days. The drugs were then released from drug treatment and cultured for three days or extended period as indicated. The senescent cells were labelled with SPiDER-0 Gal Cellular Senescence Detection Kit (Dojindo, Cat. No: SG02-10) following the manufacture's instruction. Both senescent and non-senescent cells were sorted using flow cytometry.

### Secreted Cytokine Assay

For cytokine-array analysis, cells were washed once and cultured in serum-free medium for 48 hours (Nacarelli et al., 2019). Conditioned medium was filtered (0.2 μm) and then subjected to cytokine-array assay using Human Cytokine Array C1 kit (RayBiotech. Cat. No: AAH-CYT-1-2) following the manufacturer's guidelines. After collection of conditional media, the cell number of each sample was counted. The intensities of array dots were visualized on film after incubation with SuperSignal West Pico PLUS Chemiluminescent Substrate (Thermo Fisher Scientific. Cat. No: 34580). The integrated density was measured using Image J and normalized to the cell number from which the conditioned medium was generated.

### Antibody Array

Mouse Cytokine Array C1 kit (RayBiotech. Cat. No: AAM-CYT-1-2) was used for cytokine analysis following the manufacturer's guidelines. Briefly, cells were washed once and cultured in serum-free medium for 48 hrs. Conditioned medium was filtered (0.2 μm) and then subjected to cytokine-array analysis. After collection of conditional media, the cell number of each sample was counted. The intensities of array dots were visualized on film after incubation with SuperSignal West Pico PLUS Chemiluminescent Substrate (Thermo Fisher Scientific. Cat. No: 34580). The integrated density was measured using Image J and normalized to the cell number from which the conditioned medium was generated.

2′ 3′-cGAMP Measurement

2′ 3′-cGAMP ELISA Kit (Cayman chemical. Cat. No:501700) was used to analyze the endogenous level of 2′ 3′-cGAMP following the manufacturer's instructions. 1×10⁵ IMR90 cells were incubated in 200 μL lysis buffer (ThermoFisher, Cat. No: 78501) on ice for 30 minutes. The 2′ 3′-cGAMP ELISA was performed following the manufacturer's instructions.

### Immunofluorescence

Cells were fixed with 4% paraformaldehyde (PFA) for 15 mins at room temperature followed by permeabilization with 0.2% Triton X-100 in PBS for 5 min. For DNase I digestion, after fixation and permeabilization, cells were treated with 500 units/mL RNase-Free DNase I (Qiagen, Cat. No: 79254) for one hour at 37° C. After blocking with 1% BSA in PBS, cells were incubated with primary antibody overnight at 4° C. and Alexa-Fluor conjugated secondary antibody (Life Technologies). Fluorescent images were captured using Leica TCS SP5 II scanning confocal microscope.

### Immunoblotting and Immunoprecipitation

Cells were lysed in 1× sample buffer (2% SDS, 10% glycerol, 0.01% bromophenol blue, 62.5 mM Tris, pH 6.8, and 0.1 M DTT) and heated to 95° C. for 10 min. Protein concentrations were determined using the protein assay dye (Bio-Rad. Cat. No: #5000006) and Nanodrop. An equal amount of total protein was resolved using SDS-PAGE gels and transferred to PVDF membranes at 110 V for 2 hours at 4° C. Membranes were blocked with 5% nonfat milk in TBS containing 0.1% Tween 20 (TBS-T) for 1 hour at room temperature. Membranes were incubated overnight at 4° C. in the primary antibodies in 4% BSA/TBS+0.025% sodium azide. Membranes were washed four times in TBS-T for 5 min at room temperature, after which they were incubated with HRP-conjugated secondary antibodies (Cell Signaling Technology. Cat. No: 7076S, 7074S) for 1 hour at room temperature. After washing four times in TBS-T for 5 min at room temperature, proteins were visualized on film after incubation with SuperSignal West Pico PLUS Chemiluminescent Substrate (Thermo Fisher Scientific. Cat. No: 34580).

### Antibody Information:

anti-Topoisomerase I-DNA Covalent Complexes (TOP1cc) (clone 1.1A) (Millipore. Cat. No: MABE1084), 1:1000 for slot blot
anti-TOP1 (Proteintech. Cat. No: 20705-1-AP), 1:1000 for immunoblotting
anti-cGAS (D9) (Santa Cruz. Cat. No: sc-515777), 1:1000 for immunoblotting;
anti-Cyclin A (H432) (Santa Cruz. Cat. No: sc-751), 1:1000 for immunoblotting;
anti-βactin (Sigma. Cat. No: A2228), 1:10000 for immunoblotting.

For immunoprecipitation, cells were collected and washed once with ice-cold PBS. Whole-cell extracts were lysed with RIPA buffer (50 mM Tris-HCl pH 7.4, 1% NP-40, 150 mM NaCl, 1 mM EDTA, 10% sodium deoxycholate, freshly added with 1 mM phenylmethylsulfonyl fluoride (PMSF), and cOmplete™, EDTA-free Protease Inhibitor Cocktail (Roche. Cat. No: C762Q77)). After 12,000×g centrifuge for 15 min, the supernatant was collected and incubated with antibody or isotype IgG control (5 μg per sample) at 4° C. overnight, followed by addition of 10 μL of protein A/G-conjugated agarose beads mixture (ThermoFisher. Cat. No: 10002D and 10004D). The precipitates were washed 4 times with ice-cold RIPA buffer, resuspended in 2×Laemmle buffer, and resolved by SDS-PAGE followed by immunoblotting.

### GST Pull Down

GST pull-down assay was carried out by incubating equal amounts of GST or GST-tagged cGAS (Addgene, Cat. No: 108676) that are immobilized on glutathione-sepharose beads (GE Healthcare Cat. No: 17-0756-01) with in vitro translated His-tagged TOP1 (Sino Biological, Cat. No: 17455-H07B) at 4° C. for 16 hours. Precipitated proteins were washed 3 times with elution buffer including 150 mM NaCl, eluted with SDS sample buffer, and subjected to immunoblot analysis.

Quantification PCR with Reverse Transcription

Total RNA was isolated using Trizol (Invitrogen) according to the manufacturer's instruction. Extracted RNAs were used for reverse-transcriptase PCR (RT-PCR) with High-Capacity cDNA Reverse Transcription Kit (Thermo fisher, Cat. No: 4368814). Quantitative PCR (qPCR) was performed using iTaq™ Universal SYBR® Green Supermix (BIO-RAD, Cat. No: 1725121) and QuantStudio 3 Real-Time PCR System.

The primers sequences used for quantitative RT-PCR are as follows:

| | Human IL1α |
|---|---|
| (SEQ ID NO: 4) | |
| | (forward: 5′-AGGAGAGCCGGGTGACAGTA-3′, |
| (SEQ ID NO: 5) | |
| | reverse: 5′-TCAGAATCTTCCCGTTGCTTG-3′); |
| | Human IL1β |
| (SEQ ID NO: 6) | |
| | (forward: 5′-AGCTCGCCAGTGAAATGATGG-3′, |
| (SEQ ID NO: 7) | |
| | reverse: 5′-GTCCTGGAAGGAGCACTTCAT-3′); |
| | Human IL6 |
| (SEQ ID NO: 8) | |
| | (forward: 5′-ACATCCTCGACGGCATCTCA-3′; |
| (SEQ ID NO: 9) | |
| | reverse: 5′-TCACCAGGCAAGTCTCCTCA-3′); |
| | Human IL8 |
| (SEQ ID NO: 10) | |
| | (forward: 5′-GCTCTGTGTGAAGGTGCAGT-3′; |
| (SEQ ID NO: 11) | |
| | reverse: 5′-TGCACCCAGTTTTCCTTGGG-3′); |
| | Human CXCL10 |
| (SEQ ID NO: 12) | |
| | (forward: 5′-CCATTCTGATTTGCTGCCTTATC-3′; |
| (SEQ ID NO: 13) | |
| | reverse: 5′-TACTAATGCTGATGCAGGTACAG-3′); |
| | Human CCL5 |
| (SEQ ID NO: 14) | |
| | (forward: 5′-CCAGCAGTCGTCTTTGTCAC-3′; |
| (SEQ ID NO: 15) | |
| | reverse: 5′-CTCTGGGTTGGCACACACTT-3′); |
| | Human ISG15 |
| (SEQ ID NO: 16) | |
| | (forward: 5′-GAGCATCCTGGTGAGGAATAAC-3′; |
| (SEQ ID NO: 17) | |
| | reverse: 5′-CGCTCACTTGCTGCTTCA-3′); |
| | Human B2M |
| (SEQ ID NO: 18) | |
| | (forward: 5′-GGCATTCCTGAAGCTGACA-3′; |
| (SEQ ID NO: 19) | |
| | reverse: 5′-CTTCAATGTCGGATGGATGAAAC-3′). |
| | Mouse IL1α |
| (SEQ ID NO: 20) | |
| | (forward: 5′-CCAGAAGAAAATGAGGTCGG-3′, |
| (SEQ ID NO: 21) | |
| | reverse: 5′-AGCGCTCAAGGAGAAGACC-3′); |
| | Mouse IL1β |
| (SEQ ID NO: 22) | |
| | (forward: 5′-TGTGCAAGTGTCTGAAGCAGC-3′, |
| (SEQ ID NO: 23) | |
| | reverse: 5′-TGGAAGCAGCCCTTCATCTT-3′); |
| | Mouse IL6 |
| (SEQ ID NO: 24) | |
| | (forward: 5′-GCTACCAAACTGGATATAATCAGGA-3′; |
| (SEQ ID NO: 25) | |
| | reverse: 5′-CCAGGTAGCTATGGTACTCCAGAA-3′); |
| | Mouse CXCL15 |
| (SEQ ID NO: 26) | |
| | (forward: 5′-AGAGGCTTTTCATGCTCAACA-3′; |
| (SEQ ID NO: 27) | |
| | reverse: 5′-CCATGGGTGAAGGCTACTGT-3′); |
| | Mouse CCL5 |
| (SEQ ID NO: 28) | |
| | (forward: 5′-CCACTTCTTCTCTGGGTTGG-3′; |
| (SEQ ID NO: 29) | |
| | reverse: 5′-GTGCCCACGTCAAGGAGTAT-3′); |
| | Mouse CXCL10 |
| (SEQ ID NO: 30) | |
| | (forward: 5′-TCAGCACCATGAACCCAAG-3′; |
| (SEQ ID NO: 31) | |
| | reverse: 5′-CTATGGCCCTCATTCTCACTG-3′); |
| | Mouse IL8 |
| (SEQ ID NO: 32) | |
| | (forward: 5′-AGAGGCTTTTCATGCTCAACA-3′; |
| (SEQ ID NO: 33) | |
| | reverse 5′-CCATGGGTGAAGGCTACTGT-3′); |
| | and |
| | Mouse B2M |
| (SEQ ID NO: 34) | |
| | (forward: 5′-AGTTAAGCATGCCAGTATGGCCGA-3′; |
| (SEQ ID NO: 35) | |
| | reverse: 5′-ACATTGCTATTTCTTTCTGCGTGC-3′). |
| | CCF purification |

A CCF purification protocol was developed by modifying previous protocols (Shimizu et al., 1996; Ly et al., 2017). Briefly, 500 million senescent cells were collected, resuspended, and incubated in DMEM containing 10 μg/mL cytochalasin B for 30 minutes at 37° C. After wash once with ice-cold PBS, the cell pellet was gently dounce homogenized in ice-cold pre-chilled lysis buffer (10 mM Tris-HCl, 2 mM magnesium acetate, 3 mM CaCl₂), 0.32 M sucrose, 0.1 mM EDTA, 1 mM DTT, 0.1% NP-40, 0.15 mM spermine, 0.75 mM spermidine, 10 μg/ml cytochalasin B, pH 8.5, 4° C.) with ten slow strokes of a loose-fitting pestle. Release of nuclei was confirmed by DAPI-staining and microscopy. The homogenate was fixed with 1% formaldehyde for 10 minutes, and mixed well with an equal volume of 1.6M sucrose buffer (10 mM Tris-HCl, 5 mM magnesium acetate, 0.1 mM EDTA, 1 mM DTT, 0.3% BSA, 0.15 mM spermine, 0.75 mM spermidine, pH 8.0, 4° C.). A 10 mL portion of homogenate was layered on the top of sucrose buffer gradient (20 mL and 15 mL containing 1.8M and 1.6M of sucrose, respectively) in a 50 mL tissue culture tube. The tubes were centrifuged at 1200×g for 20 minutes at 4° C. After centrifugation, the upper 3 mL of the gradient was discarded, and the next 15 mL containing CCFs was collected. The collected fraction was diluted with an equal volume of ice-cold PBS, and filtered through 5 μm low protein binding durapore (PVDF) membrane (Millipore. Cat. No: SLSV025LS) to remove the contaminated nuclei. DAPI-staining was performed at this step to confirm the clearance of contaminated nuclei. The CCF fractions were diluted 5-fold by adding ice-cold PBS, then centrifuged at 2000×g for 15 minutes at 4° C. Finally, the pellet was suspended in 200 μL ice-cold PBS buffer. The CCF samples were broken down by one pulse of bioruptor with high output. DNA concentration was measured using Nanodrop and 5 μg DNA was used for slot blot analysis.

### BrdU Incorporation Assay and IMMUNOFLUORESCENCE

Cells were plated on coverslips and labelled with 10 μg/ml BrdU for 24 hrs. Cells were fixed with 4% paraformaldehyde (PFA) for 15 mins at room temperature followed by permeabilization with 0.2% Triton X-100 in PBS for 5 min. Cells were incubated in 2.5M hydrochloric acid at 4° C. for 24 hrs. After blocking with 1% BSA in PBS, cells were incubated with primary antibody overnight at 4° C. and Alexa-Fluor conjugated secondary antibody (Life Technologies) for one hr. Fluorescent images were captured using Leica TCS SP5 II scanning confocal microscope.

### SILAC-MS Analysis

SILAC DMEM Lysine (6) Arginine (10) Kit (Silantes. Cat No: 282986434) was used for the SILAC-MS analysis. Briefly, IMR90 cells were cultured in “heavy” medium containing ¹³C₆ labeled lysine and ¹³C₆, ¹⁵N₄ labeled arginine, or “light” medium containing unlabeled lysine and arginine for at least four passages. The “heavy” labeled IMR90 cells were infected with short hairpin control lentivirus, and the “light” cells were infected with shHMGB2 short hairpin lentivirus (TRCN0000019011). After puromycin selection, the cells were treated with 50 μM Etoposide for 2 days. After washing off the drug with fresh medium, the treated cells were cultured for 6 days to induce senescence. The same numbers of both “heavy” and “light” labeled cells were mixed together and the CCF purification was performed. Purified CCF were mixed with 5×SDS sample buffer and boiled at 95° C. for 15 minutes.

LC-MS/MS analysis was performed using a Q Exactive HF mass spectrometer (ThermoFisher Scientific) coupled with a Nano-ACQUITY UPLC system (Waters). Samples were digested with trypsin and tryptic peptides were separated by reversed phase HPLC on a BEH C18 nanocapillary analytical column (75 μm i.d.×25 cm, 1.7 μm particle size; Waters) using a 240 min gradient formed by solvent A (0.1% formic acid in water) and solvent B (0.1% formic acid in acetonitrile). Eluted peptides were analyzed by the mass spectrometer set to repetitively scan m/z from 400 to 2000 in positive ion mode. The full MS scan was collected at 60,000 resolution followed by data-dependent MS/MS scans at 15,000 resolution on the 20 most abundant ions exceeding a minimum threshold of 10,000. Peptide match was set as preferred, exclude isotope option and charge-state screening were enabled to reject unassigned, and single charged ions. The sample was analyzed twice (technical replicate). Peptide sequences were identified using MaxQuant 1.6.2.3²⁹. MS/MS spectra were searched against a UniProt human protein database (October 2017) and a common contaminants database using full tryptic specificity with up to two missed cleavages, static carboxamidomethylation of Cys, and variable oxidation of Met, and protein N-terminal acetylation. Consensus identification lists were generated with false discovery rates set at 1% for protein and peptide identifications. The DAVID bioinformatics resources 6.8 was used for functional classification analysis. The protein list was further filtered to include only proteins classified as “nucleosome and chromosome related” and identified by at least two razor+unique peptides with a minimum absolute fold change of 1.2 in both replicates.

### Chromatin Fragment Extraction and Transfection

For chromatin fragments extraction, proliferating IMR90 cells were treated with 5 μM or 50 μM Camptothecin for 30 minutes to induce low or high levels of TOP1cc, respectively. Cells then were incubated with hypotonic buffer (10 mM Tris, pH 7.4, 30 mM NaCl, 3 mM MgCl₂, 0.1% NP40), supplemented with protease inhibitor cocktail, on ice for 10 mins (Dou et al., 2017). The cells were then centrifuged at 300×g for 3 mins at 4° C. The supernatant was carefully removed, and the resulting pellets were incubated with benzonase buffer (50 mM Tris pH 7.5, 300 mM NaCl, 0.5% NP40, 2.5 mM MgCl₂) with protease inhibitor cocktail, supplemented with 10 U of benzonase (Sigma Cat. No: E1014), on ice, for 30 min. The product was centrifuged again at 300×g for 3 mins at 4° C., and benzonase was inactivated by addition of 15 mM EDTA. The resulting supernatant contains chromatin fragments and soluble nuclear proteins. For the negative controls, buffer without benzonase was used and the resulting supernatant only contains soluble nuclear proteins without chromatin fragments. The product was then diluted 5 times with PBS. Slot blot were performed to confirm the TOP1cc level. The chromatin fragments or negative controls were transfected into proliferating IMR90 cells using lipofectamine 2000. Successful transfection was confirmed by immunofluorescence with DAPI staining. Transfected cells were harvested 4 days post transfection and were used for RT-qPCR or immunofluorescence analysis.

TOP1 ICE (In vivo Complex of Enzyme) Assay

Human Topoisomerase 1 ICE Assay Kit (TopoGEN. Cat. No:TG1020-1) was used to isolate protein-DNA samples which contain TOP1-DNA covalent complex (TOP Ice) for slot blot analysis. The isolation was performed following the manufacturer's guidelines. 5×10⁵ cells were used for ICE assay and TOP1cc analysis. Purified CCF samples were sonicated and used for slot blot directly. Briefly, cells were lysed with 300 μL of room temperature buffer A, and then 115 μL buffer B was added to precipitate DNA. After washing with buffer C, DNA was dissolved in buffer D and buffer E. The DNA samples were kept in 37° C. to promote the recovery. Nano-Drop was used to measure the DNA concentration. 5 μg DNA was used for each slot blot analysis. Bio-Dot SF Microfiltration Apparatus (Bio Rad. Cat. No:1706542) was used for slot blot. Quantification was performed using NIH Image J software.

### Electrophoretic Mobility-Shift (EMSA) Assays

EMSA was performed as previously described (Li et al., 2013; Liu et al., 2019). Briefly, recombinant cGAS was incubated, in the presence or absence of recombinant TOP1 or TOP1Y723F mutant, with (ISD)₂ dsDNA in the cGAMP synthesis buffer at 37° C. for 30 mins. The mixtures were loaded on 1% agarose gel using an electrophoresis buffer (40 mM Tris-HCl at pH 10.5). The gels were then stained with SYBR™ Green I Nucleic Acid Gel Stain and images were acquired using UV Transilluminator (Analytik Jena).

### In Vivo Orthotopic Syngeneic Mouse Model

The protocols were approved by the Institutional Animal Care and Use Committee of the Wistar Institute. Results from in vitro experiments were used to determine the in vivo sample size. For orthotopic syngeneic model, luciferase expressing ID8-Defb29/Vegf-a with inducible shHMGB2 cells were pretreated with 2 μM Cisplatin for 48 hours to induce CCFs. 5×10⁶ cells were i.p. injected into the peritoneal cavity of C57BL/6 mouse (female, 6-8 weeks old, CRL/NCI) (Zhu et al., 2016). Animals were randomly assigned to different treatment groups (10 mice/group). The mice in control groups were fed with control rodent diet (Fisher Scientific. Cat. No: 14-726-309). For the HMGB2 knockdown groups, mice were fed with Bio-Serv™ Doxycycline Grain-Based Rodent Diet (Fisher Scientific. Cat. No: 14-727-450) to induce HMGB2 knockdown. Tumor progression was monitored twice a week using a Xenogen IVIS Spectrum in vivo bioluminescence imaging system. Images were analyzed using Live Imaging 4.0 software. Tumor-bearing mice were treated by i.p. injection with isotype control IgG or anti-PD-L1 antibody (Bio X Cell, Cat. No: B7-H1, clone 10 F.9G2, 10 mg kg\hat{ }-1) every 3 days with or without simultaneous TOP1 inhibitor camptothecin treatment (Selleck Cat. No: S1288; 8 mg kg\hat{ }-1). For survival analysis, the Wistar Institute IACUC guideline was followed in determining the time for ending the survival experiments (tumor burden exceeds 10% of body weight).

For peritoneal wash, the peritoneal cavity of mice was washed three times with 5 ml PBS. Single-cell suspensions were prepared, and red blood cells were lysed using ACK Lysis Buffer (Thermo Fisher, Cat No: A1049201). Live/dead cell discrimination was performed using Zombie Yellow™ Fixable Viability Kit (Biolegend, Cat No: 423104). Cell surface staining was done for 30 mins at 4° C. using antibodies against CD3ε (Biolegend, Cat No: 423104), CD69 (Biolegend, 104525), CD8 (Biolegend, Cat No: 100708), CD4 (Biolegend, Cat No: 100516), Granzyme B (Biolegend, Cat No: 372206), and Interferon gamma (Biolegend, Cat No: 505825). Intracellular staining was done using an eBioscience fixation/permeabilization kit (Thermo Fisher, Cat No: 88-8824-00). All data acquisition was done using an LSR II (BD) or FACSCalibur (BD) and analyzed using FlowJo software (TreeStar) or the FlowCore package in the R language and environment for statistical computing.

### In Vivo Mouse Model and Profiling of Infiltrated Immune Cells

The protocols were approved by the Institutional Animal Care and Use Committee of the Wistar Institute. 1×10⁶ UPK10 cells were unilaterally injected into the ovarian bursa sac of C57BL/6 mouse (female, 6-8 weeks old, CRL/NCI). The orthotopically transplanted cells were allowed to form tumor for 15 days. Tumor-bearing mice were randomly assigned to different treatment groups. The mice were treated for two weeks. Specifically, the mice were pre-treated by i.p. injection (1×106cells per mouse) with control UPK10 cells (group 3), or senescent UPK10 cells sorted from cisplatin, irinotecan and cisplatin/irinotecan combination treated groups (group 4, 5 and 6), or 10 mg/kg DMXAA (group 8), or DMSO vehicle control (group 7). 24 hrs following the pre-treatment, the mice were treated by i.p. injection with anti-PD-1 antibody (Bio X Cell, Cat. No: BE0273, clone 29F.1A12, 10 mg/kg) or an isotype matched IgG control every 3 days. After two weeks of treatment, the tumors were collected and digested using mixture of 10 mg/mL Collagenase (Sigma, Cat No: C5138), 1 mg/mL Hyaluronidase (Sigma, Cat No: H3884) and 200 mg/mL DNase 1 (Sigma, Cat No: D5025) at 37° C. for 1 hr. Single-cell suspensions were prepared, and red blood cells were lysed using ACK Lysis Buffer (Thermo Fisher, Cat No: A1049201). Live/dead cell discrimination was performed using LIVE/DEAD™ Fixable Aqua Dead Cell Stain Kit (Thermo Fisher, Cat No: L34968). Cell surface staining was done for 30 min at 4° C. All data acquisition was done using an LSR II (BD) or FACS Calibur (BD) and analyzed using FlowJo software (TreeStar) or the FlowCore package in the R language and environment for statistical computing. For survival analysis, the Wistar Institute IACUC guideline was followed in determining the time for ending the survival experiments (mice succumbed to the disease or tumor burden exceeds 10% of body weight).

### Immunofluorescence Staining for Tumor Tissue Sections

Formalin-fixed, paraffin-embedded tumors were sectioned, and slides were deparaffinized and rehydrated. Antigen retrieval was performed by boiling for 40 mins in citrate buffer, pH6.0 (Thermo Fisher). Endogenous peroxidases were quenched with 3% hydrogen peroxide in methanol. Sections were then blocked with 5% BSA/PBS at room temperature for 1 hr. Sections were incubated with primary mouse anti-GFP (Santa Cruz, 1:400 dilution) or rabbit anti-mCherry (Proteintech, 1:200 dilution) antibodies at 4° C. overnight. Detection was performed using secondary Alexa Fluor 488-conjugated goat anti-mouse IgG (Thermo Fisher, 1:1000 dilution) and Alexa Fluor 555-conjugated goat anti-rabbit IgG (Thermo Fisher, 1:1000 dilution) at room temperature for 1 hr. The sections were counter stained with DAPI containing Duolink® in Situ mounting medium (Sigma Aldrich) and sealed. Samples were imaged on Leica TCS SP5 II Scanning Confocal Microscope.

### Statistical Analysis

Results are representative of a minimum of three independent experiments. All statistical analyses were conducted using GraphPad Prism 6 (GraphPad). The Student's t-test was performed to determine P values of the raw data unless otherwise stated, where P<0.05 was considered significant. Animal experiments were randomized. There was no exclusion from the experiments.

### Example 2: Topoisomerase 1 Cleavage Complex Enables Pattern Recognition and Inflammation During Senescence

HMGB2 is Required for cGAS' Localization into CCF During Senescence

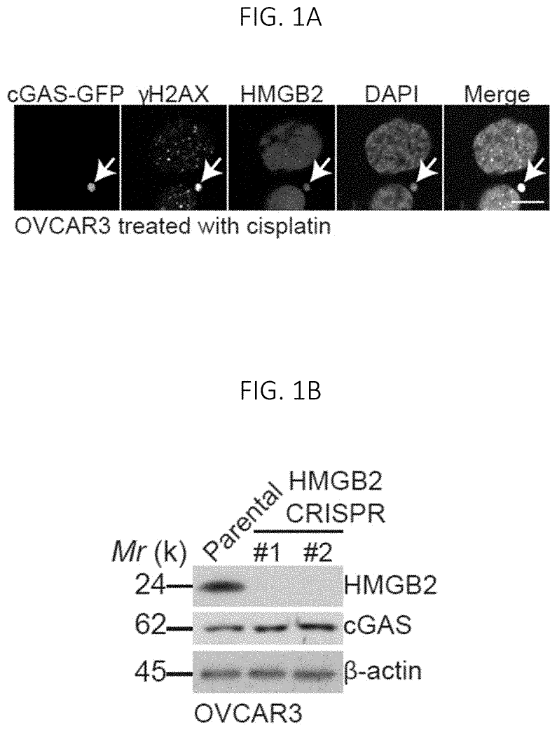

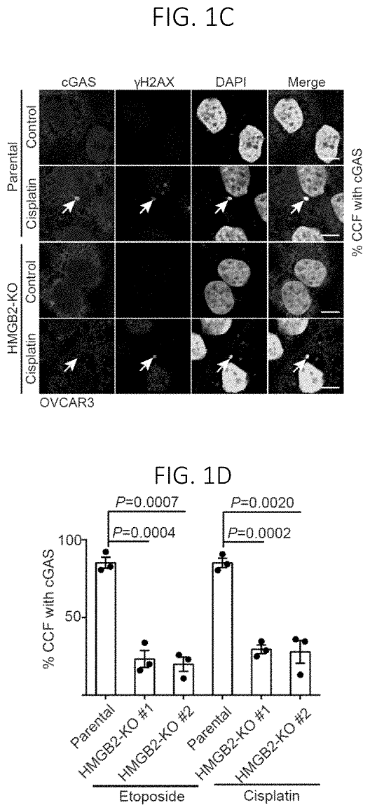

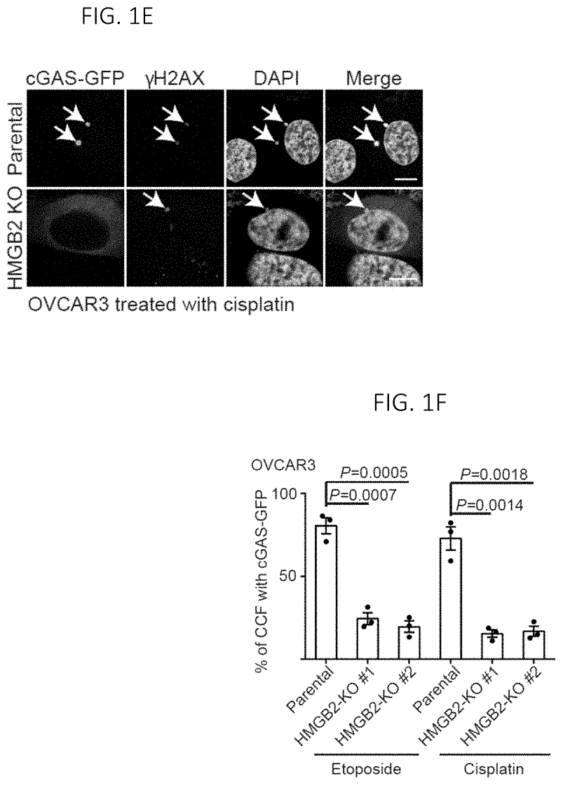

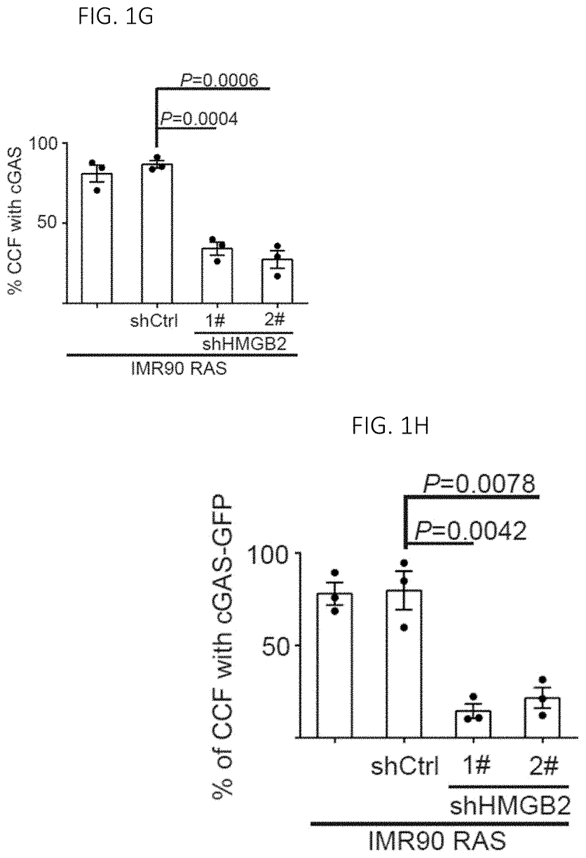

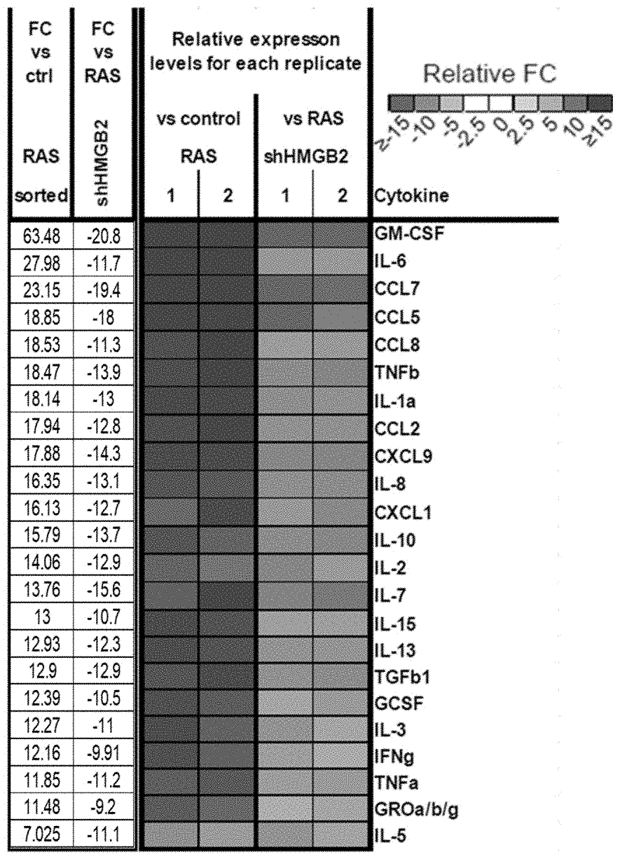

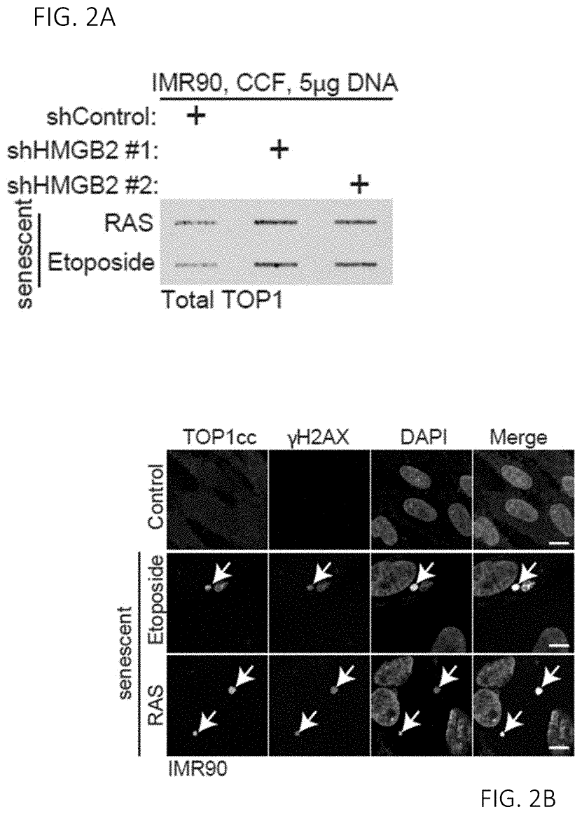

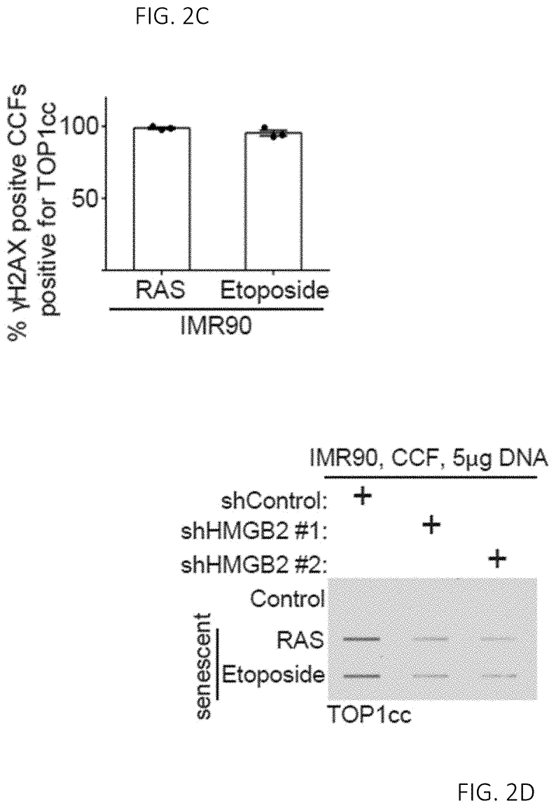

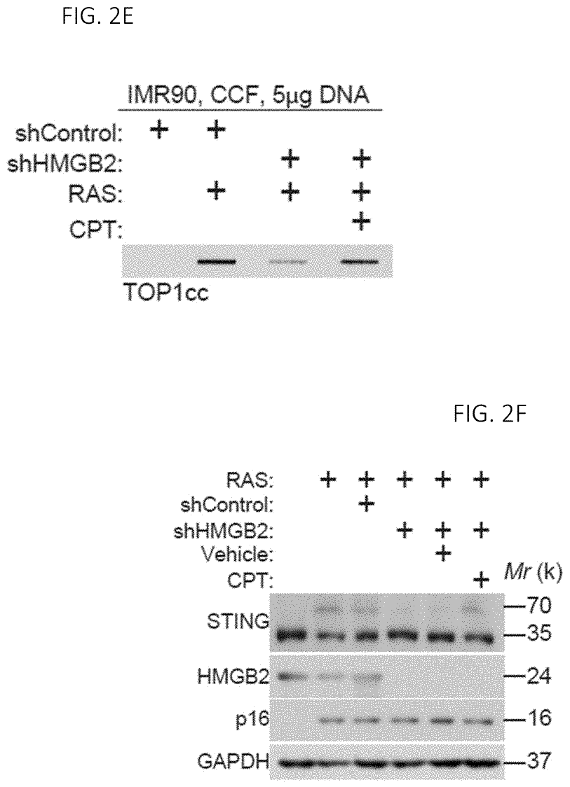

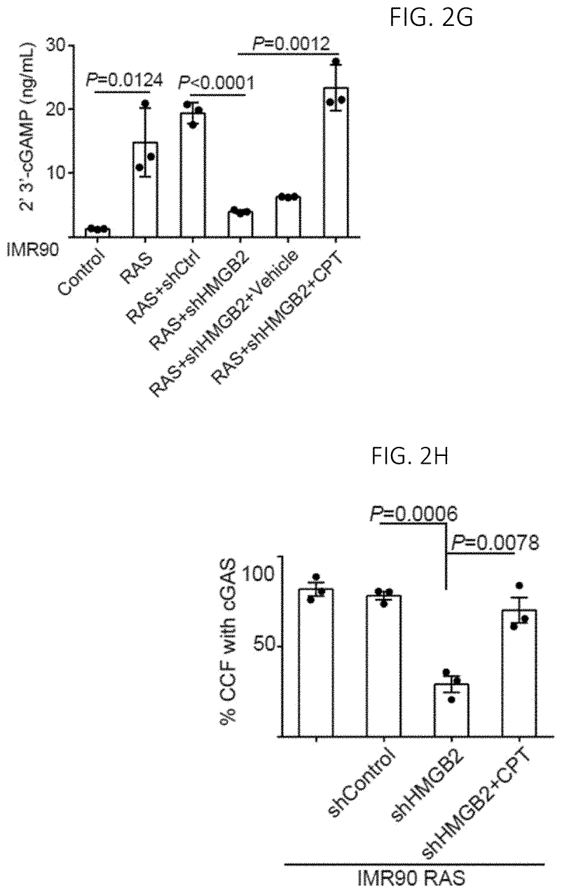

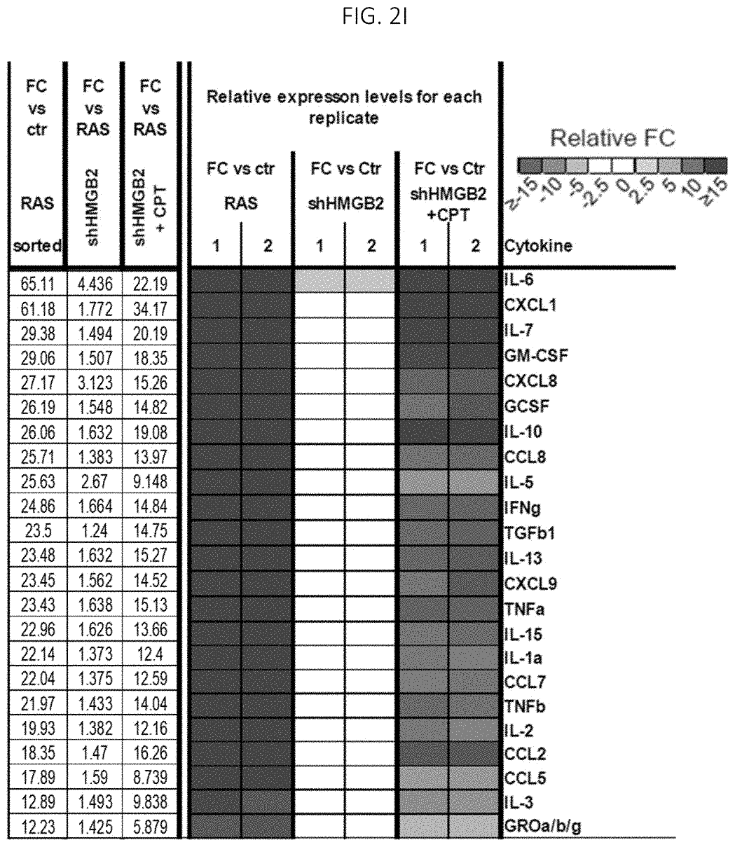

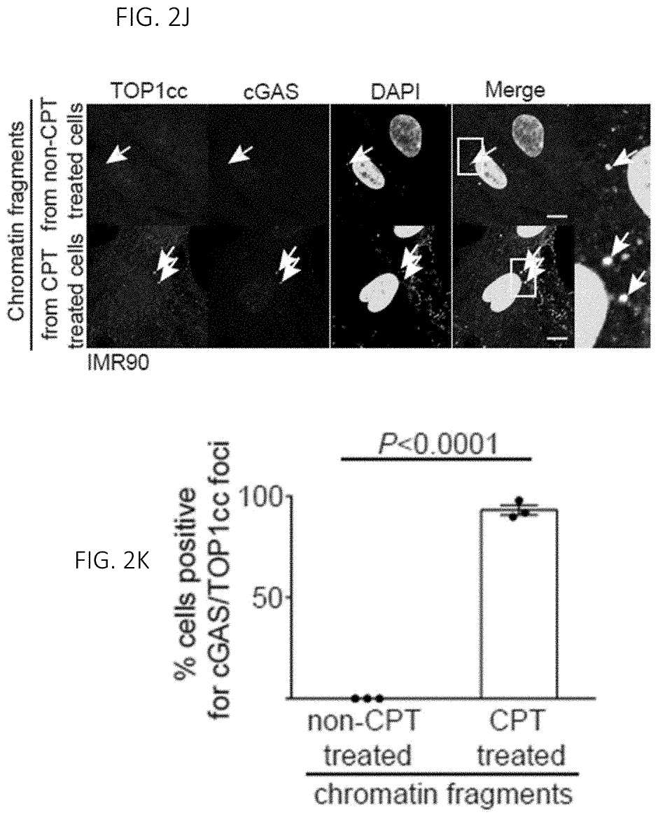

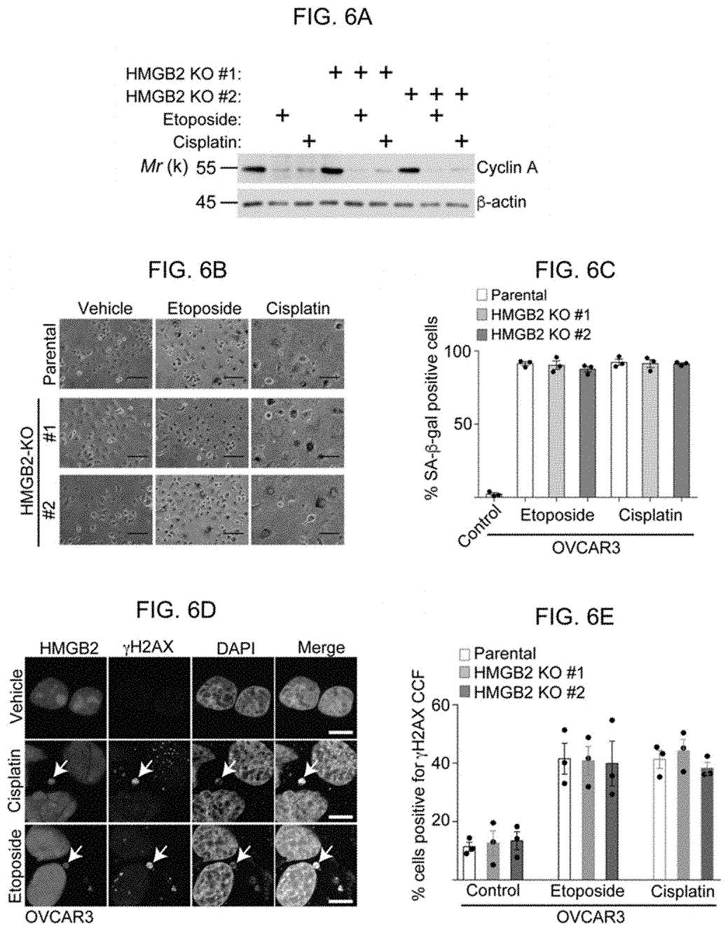

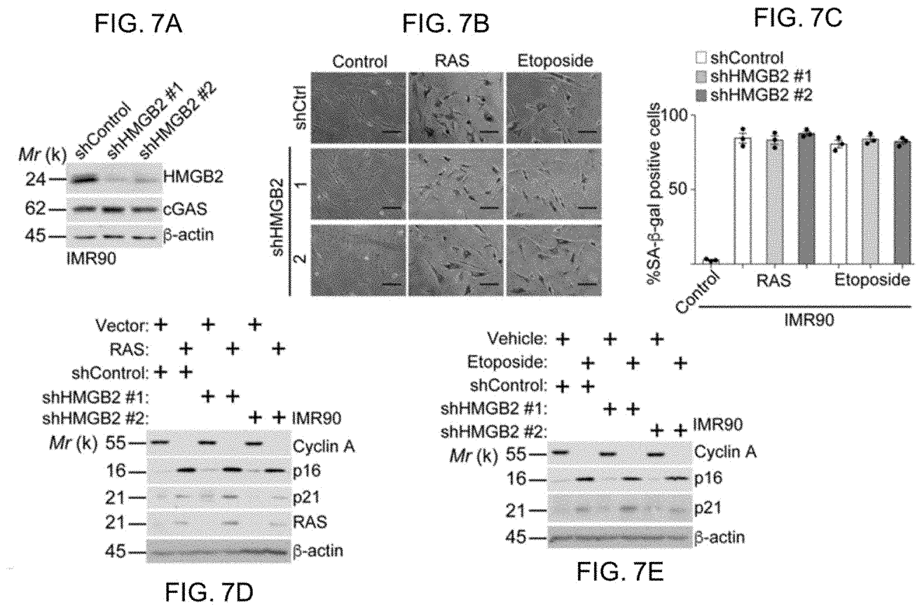

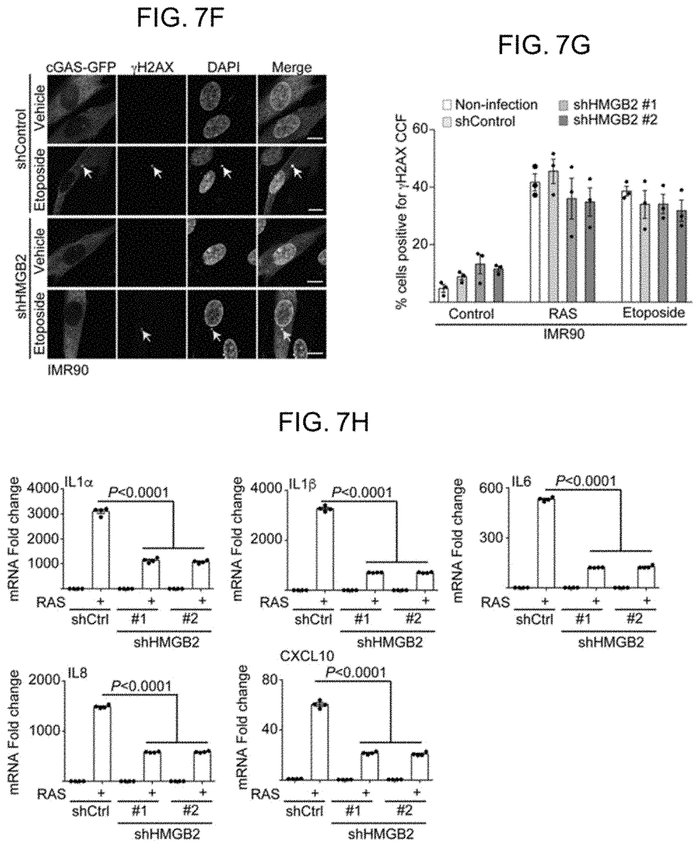

Since HMGB2 positively regulates SASP (Aird et al., 2016) and facilitates cytosolic nucleic-acid sensing (Yanai et al., 2009), we examined whether HMGB2 localized to the CCF during senescence. HMGB2 co-localized with γH2AX in the CCF in senescent OVCAR3 ovarian cancer cells induced by either cisplatin or etoposide (FIG. 6A-6D). Indeed, HMGB2 co-localized with cGAS and γH2AX in the CCF in therapy-induced senescent OVCAR3 cells (FIG. 1A). We next determined the effects of HMGB2 loss on CCF and recognition of CCF by cGAS. We generated two HMGB2 knockout OVCAR3 clones (FIG. 1B). HMGB2 knockout did not affect CCF formation as examined by γH2AX's localization to CCF (FIGS. 1C-1F and FIG. 6E). However, HMGB2 knockout significantly decreased the localization of cGAS into the CCF (FIG. 1C-1F). Consistent with previous reports that HMGB2 knockdown selectively suppresses SASP but does not affect senescence-associated growth arrest (Aird et al., 2016), HMGB2 knockout did not affect other markers of senescence such as SA-β-Gal activity and downregulation of proliferation marker cyclin A (FIG. 6A-6C). This indicates that the observed changes in cGAS localization were not a consequence of senescence suppression by HMGB2 knockout. Similar findings were also made in oncogenic H-RAS^{G12V} or etoposide-induced senescent primary embryonic lung fibroblast IMR90 cells with or without shRNA-mediated HMGB2 knockdown (FIGS. 1A-1G and FIG. 2A-2K). Notably, HMGB2 knockout or knockdown did not decrease cGAS expression (FIG. 1B and FIG. 2A), suggesting the observed loss of cGAS' localization into CCF was not due to a decrease in cGAS protein expression. Consistent with a significant decrease in cGAS localization to CCF, HMGB2 knockdown significantly decreased the levels of secreted SASP factors as determined by an antibody-based array (FIG. 1I). Likewise, mRNA expression of SASP genes was also suppressed by HMGB2 knockdown (FIG. 7H). Together, we conclude that HMGB2 localizes to CCF and is required for cGAS' localization to CCF.

cGAS Activation Requires TOP1cc During Senescence

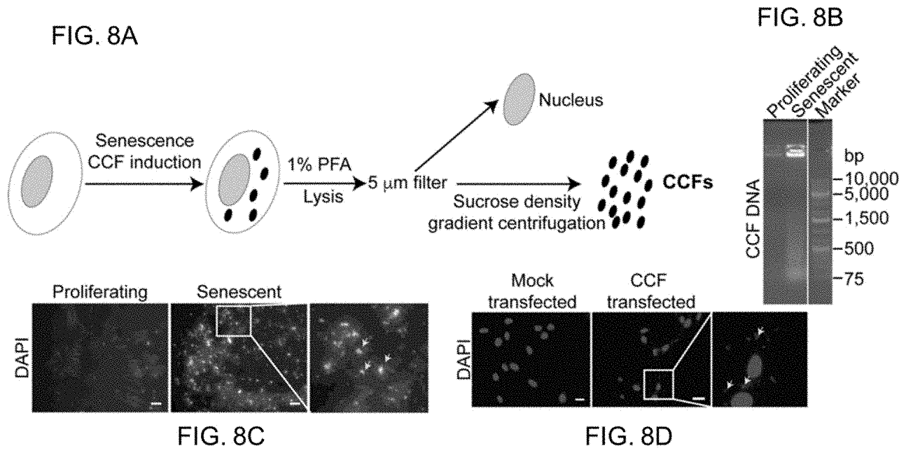

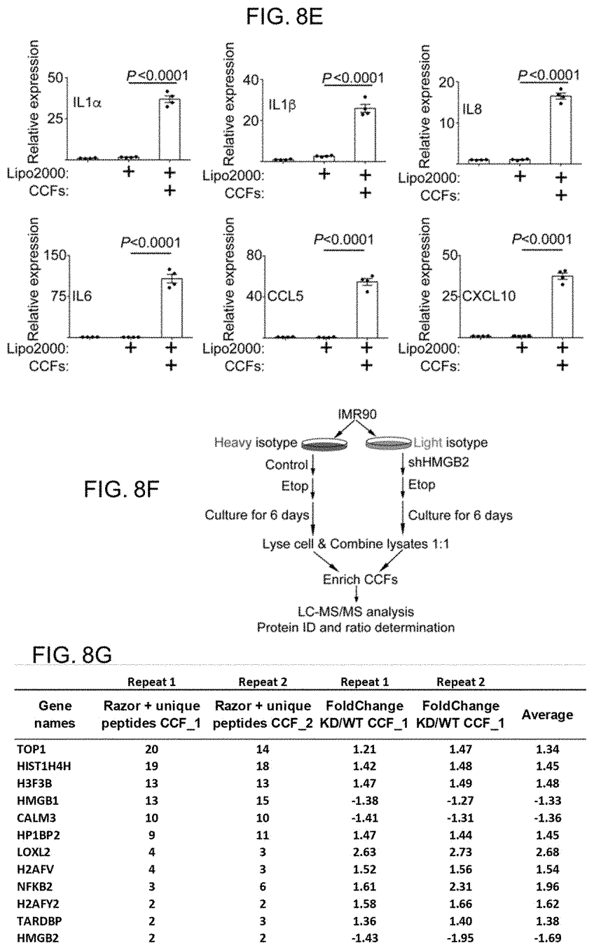

We next determined the mechanism by which HMGB2 regulates cGAS' localization into CCF during senescence. Toward this goal, we developed a protocol to purify CCF from senescent cells (FIG. 8A-8C). Transfection of the purified CCF from etoposide-induced senescent IMR90 cells upregulated the expression of SASP genes in naïve IMR90 cells, validating the protocol we developed (FIG. 8D-8E). We next performed stable isotope labeling with amino acids in cell culture (SILAC) by labelling etoposide-induced senescent IMR90 cells with or without inducible HMGB2 knockdown with light or heavy isotopes, respectively (FIG. 8F). We isolated the CCF from these cells and performed liquid chromatography tandem mass spectrometry (LC-MS) analysis to identify proteins that are differentially localized to CCF in senescent cells with vs. without HMGB2 knockdown. We focused our analysis on proteins that are implicated in the nucleosome and chromosome-related functionality given that CCF formed by nuclear membrane blebbing are positive for chromatin markers (Dou et al., 2017; Ivanov et al., 2013). The analysis revealed that topoisomerase 1 (TOP1) was among the top differentially proteins in CCF isolated from senescent cells with or without HMGB2 knockdown. TOP1 levels in CCF were increased by HMGB2 knockdown compared with control senescent cells (FIG. 8G). Notably, TOP1 forms TOP1cc without strict DNA sequence preference (Pommier et al., 2016). Thus, TOP1 exists in two forms: free TOP1 and TOP1cc covalently bound to dsDNA (Pommier et al., 2016). Notably, inhibition of TOP1 activity by camptothecin (CPT) leads to trapping of TOP1cc on DNA and thus increases TOP1cc levels (Pommier et al., 2016).

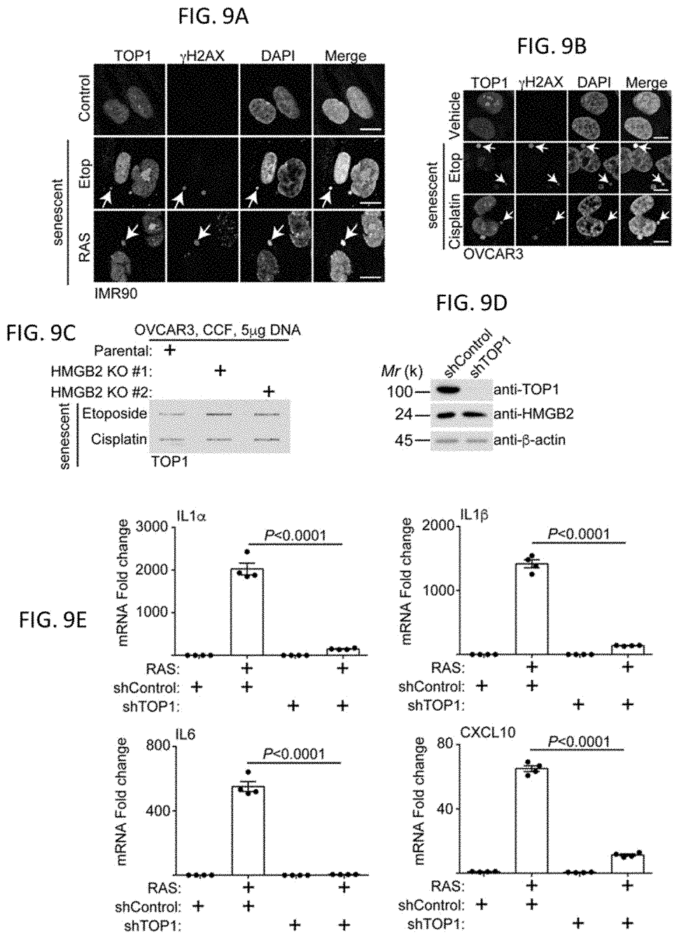

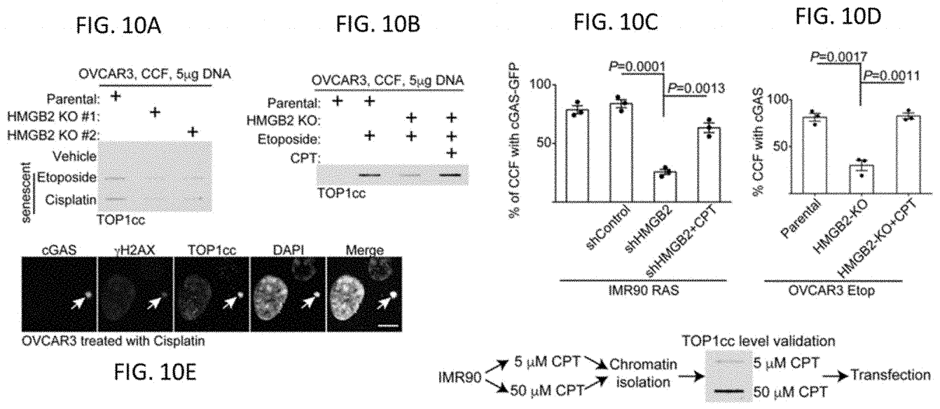

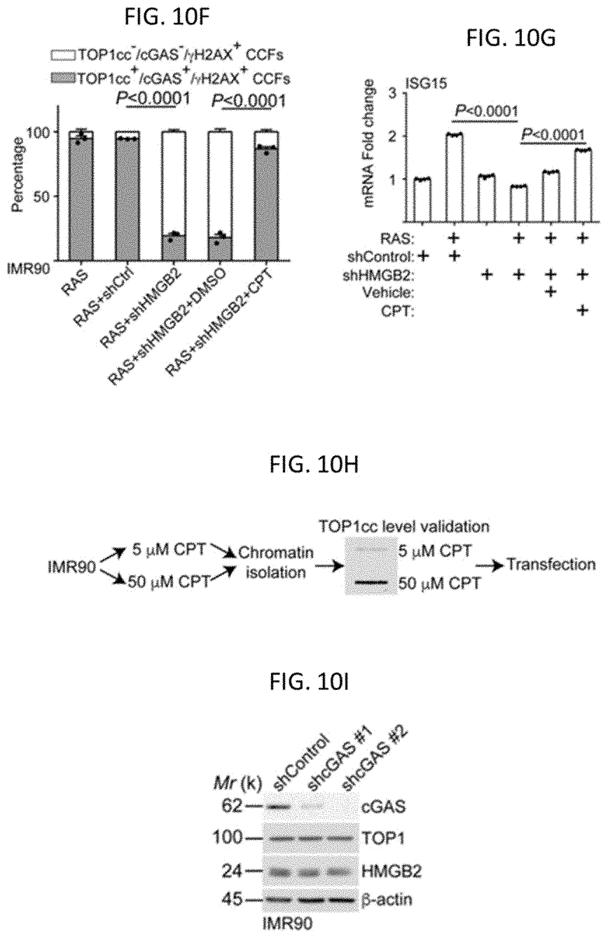

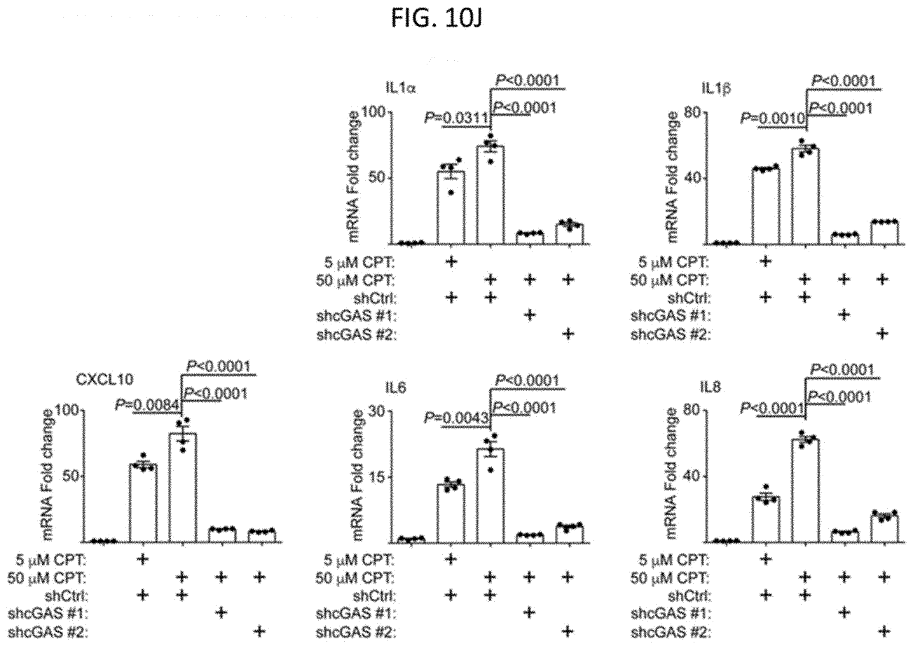

We first validated the unbiased LC-MS results by showing that TOP1 localized to CCF and co-localized with γH2AX in both senescent IMR90 and OVCAR3 cells (FIG. 9A-9B). We further validated that TOP1 levels in CCF were increased by HMGB2 knockdown in senescent IMR90 cells (FIG. 2A) and by HMGB2 knockout in senescent OVCAR3 cells (FIG. 9C). TOP1 levels in CCF were increased by HMGB2 inhibition that suppresses SASP, suggesting that TOP1 may negatively regulate SASP. However, knockdown of TOP1 significantly suppressed the expression of SASP genes (FIG. 9D-9E), suggesting that the presence of TOP1 in CCF positively regulates SASP. Thus, although TOP1 levels in CCF were increased in HMGB2-inhibited senescent cells, TOP1 may positively regulate SASP. Therefore, we instead examined the localization of TOP1cc in CCF in senescent cells with or without HMGB2 inhibition. Indeed, TOP Ice localized to CCF and co-localized with γH2AX in CCF (FIG. 2B-2C). However, in contrast to an increased level of TOP1 in CCF, TOP1cc levels in CCF were decreased by HMGB2 knockdown or knockout (FIG. 2D and FIG. 10A), which is consistent with the finding that HMGB2 loss suppresses CCF-mediated SASP (FIG. 1A-1I).

Since our results suggested that TOP1cc promotes SASP, we next directly examined whether induction of TOP1cc by CPT is sufficient to rescue the suppression of SASP induced by HMGB2 inhibition. Notably, CPT treatment restored the TOP1cc levels in the CCF isolated from HMGB2 knockdown or knockout senescent cells (FIG. 2E and FIG. 10B). In addition, CPT treatment rescued the suppression of STING dimerization and downregulation of 2′3′-cGAMP levels induced by HMGB2 knockdown (FIG. 2F-2G), which correlated with a rescue of the localization of cGAS and TOP1cc into CCF in HMGB2 knockout cells (FIG. 2H and FIGS. 10C-10F) and the restoration of the secretion of SASP factors as determined by an antibody array (FIG. 2I). Similar rescue was also observed for expression of cGAS-STING regulated type I IFN target gene ISG15 (FIG. 10G). We next determined whether TOP1cc is sufficient to drive cGAS localization into CCF and upregulate SASP genes. We isolated genomic chromatin fragments from IMR90 cells treated with two doses of CPT that induced TOP1cc in a dose-dependent manner (FIG. 10H). Transfection of the isolated TOP1cc-containing genomic chromatin fragments was indeed sufficient to induce SASP gene expression in a dose-dependent manner (FIG. 10I-10J). Notably, TOP1cc-containing genomic chromatin fragments induced the co-localization of TOP1cc and cGAS (FIG. 2J-2K). The observed SASP induction by TOP1cc-containing genomic chromatin fragments was cGAS dependent because cGAS knockdown abrogated the observed SASP induction (FIG. 10I-10J). Together, these results support that TOP1cc functions downstream of HMGB2 and upstream of cGAS.

TOP1cc Enhances dsDNA Recognition by cGAS

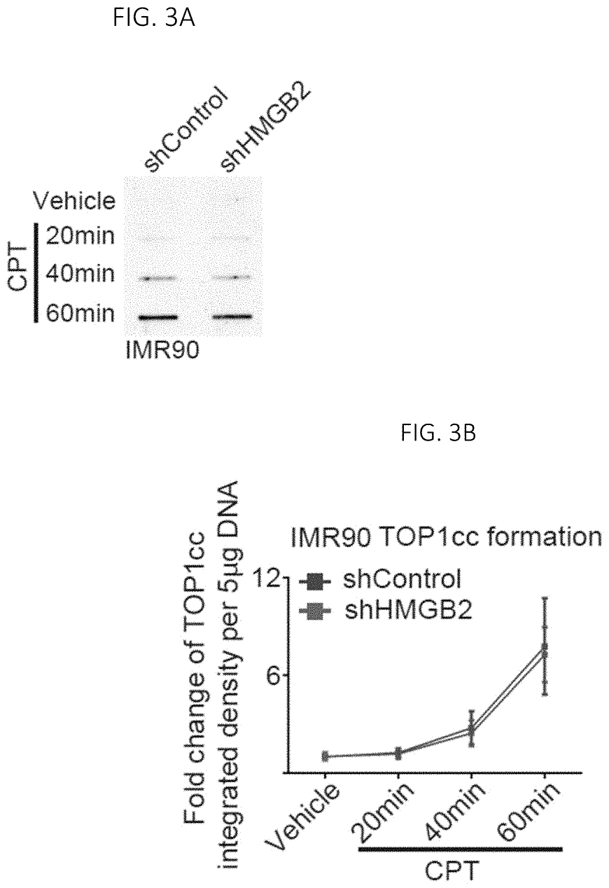

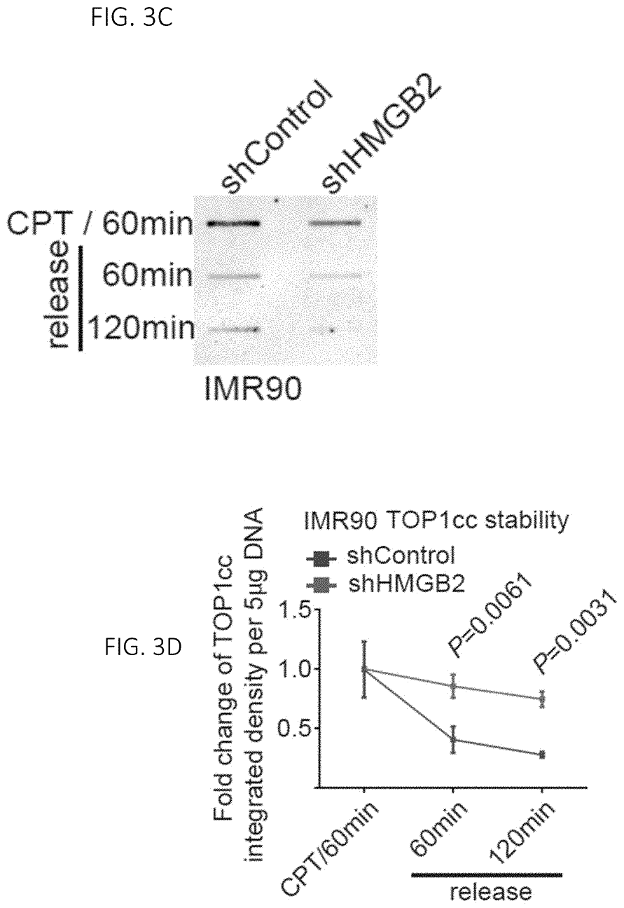

Since HMGB2 positively regulates TOP1cc and HMGB2 inhibition decreases TOP1cc, we examined time-course kinetics of TOP1cc induction and stabilization in CPT-treated IMR90 cells with or without HMGB2 knockdown. Notably, HMGB2 knockdown did not affect the kinetics of TOP Ice formation (FIG. 3A-3B). In contrast, HMGB2 knockdown significantly decreased TOP1cc levels once the cells were released from CPT treatment (FIG. 3C-3D). These results support that HMGB2 stabilizes TOP1cc.

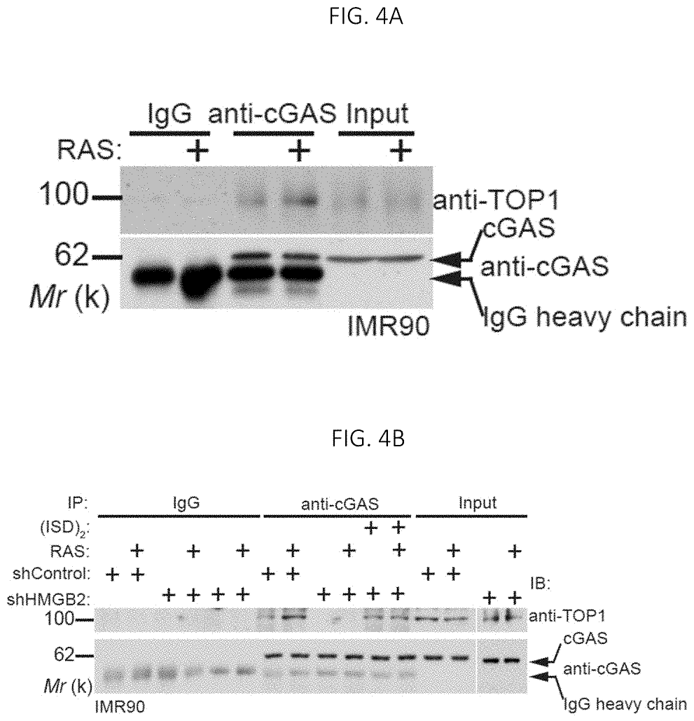

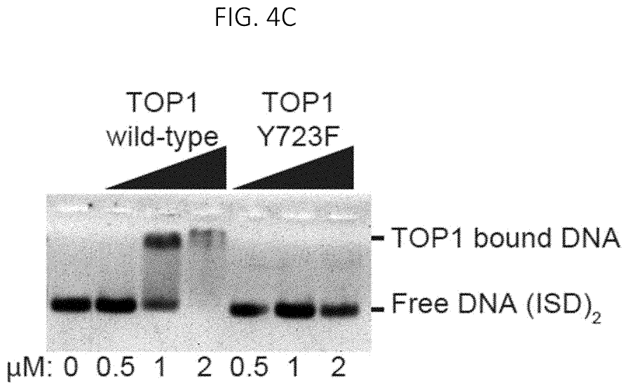

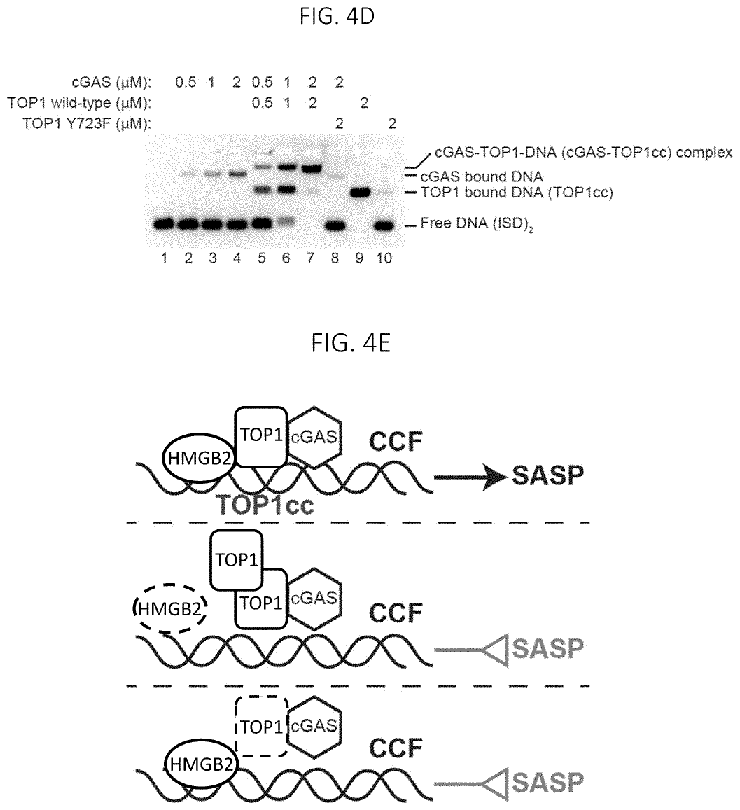

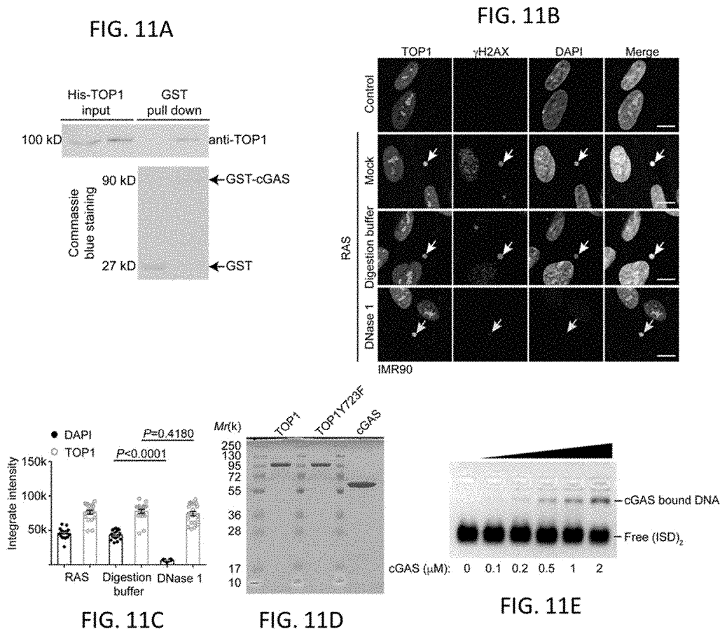

Since SASP induction by TOP1cc is cGAS dependent, TOP1cc is a TOP1 covalently modified dsDNA complex (Pommier et al., 2016), and cGAS binds to dsDNA (Sun et al., 2013), we examined whether TOP1 interacts with cGAS by co-immunoprecipitation analysis. Indeed, TOP1 interacted with cGAS and there was an increase in their interaction in senescent compared with control cells (FIG. 4A-4B). Notably, TOP1 directly interacts with cGAS in a GST-pull down assay (FIG. 11A). Interestingly, the interaction between cGAS and TOP1 is HMGB2 dependent because HMGB2 knockdown abrogated the interaction in DNA free co-IP lysates (FIG. 4B). This result suggests that cGAS interacts with TOP1cc, the DNA bound form of TOP1, because HMGB2 stabilizes TOP1cc which may explain the lack of interaction between cGAS and TOP1 in HMGB2 knockdown cells. Indeed, addition of a synthesized 45 bp interferon stimulatory dsDNA (ISD)₂ (Li et al., 2013) into to the lysates of HMGB2 knockdown cells to allow for TOP1cc formation significantly rescued the interaction between TOP1 and cGAS in HMGB2 knockdown senescent cells (FIG. 4B). Notably, DNase I treatment significantly reduced the intensity of DAPI-stained DNA in CCF (FIG. 11B-11C). However, the localization of TOP1 into the CCF was not affected by DNase I treatment (FIG. 11B-11C). This result suggests that TOP1 can localize into CCF independent of DNA, which is consistent with our findings that HMGB2 knockdown reduced the TOP1cc levels while increased TOP1 levels in CCF (FIG. 2A-2K). Together, these results show that TOP Ice interacts with cGAS and HMGB2 regulates the interaction through controlling TOP1cc stability.

We next sought to directly determine the effects of TOP1cc on the DNA binding affinity of cGAS. Electrophoretic mobility-shift assay (EMSA) showed high-molecular-weight cGAS bound (ISD)₂ dsDNA complex in dose-dependent manner (FIG. 6D-6E). In addition, EMSA showed that compared with wild-type TOP1, a point mutant TOP1 Y723F that is defective in DNA binding and thus cannot form TOP1cc, was severely impaired in its ability to shift the free (ISD)₂ dsDNA (FIG. 4C). Significantly, wild-type TOP1, but not the TOP1 Y723F mutant, markedly enhanced the dsDNA binding affinity of cGAS (FIG. 4D; lane 7 vs. 8). Together, these results support that TOP1cc formed by DNA binding wild-type TOP1 enhances dsDNA recognition by cGAS (FIG. 4E).

### HMGB2-TOP1cc-cGAS Determines Response to Checkpoint Blockade

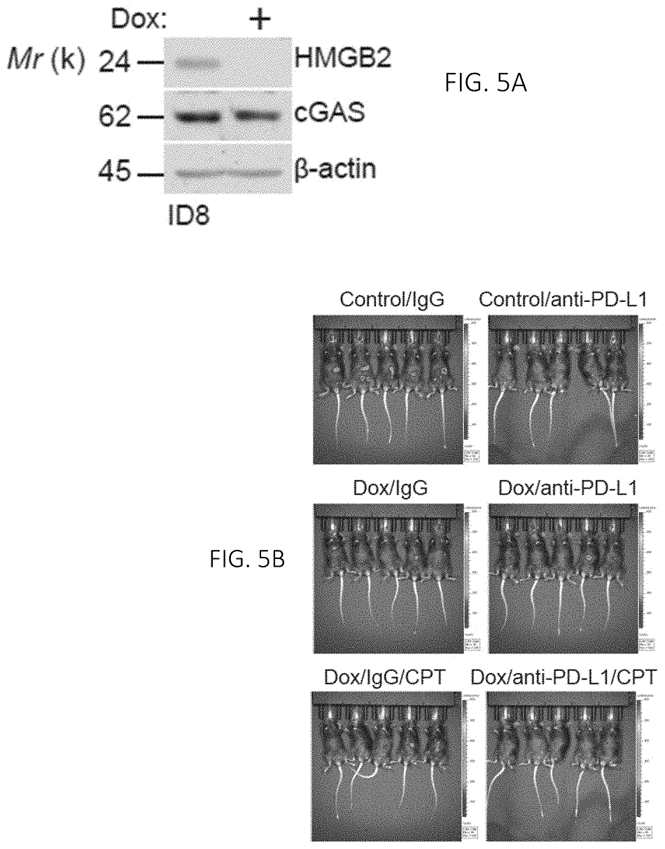

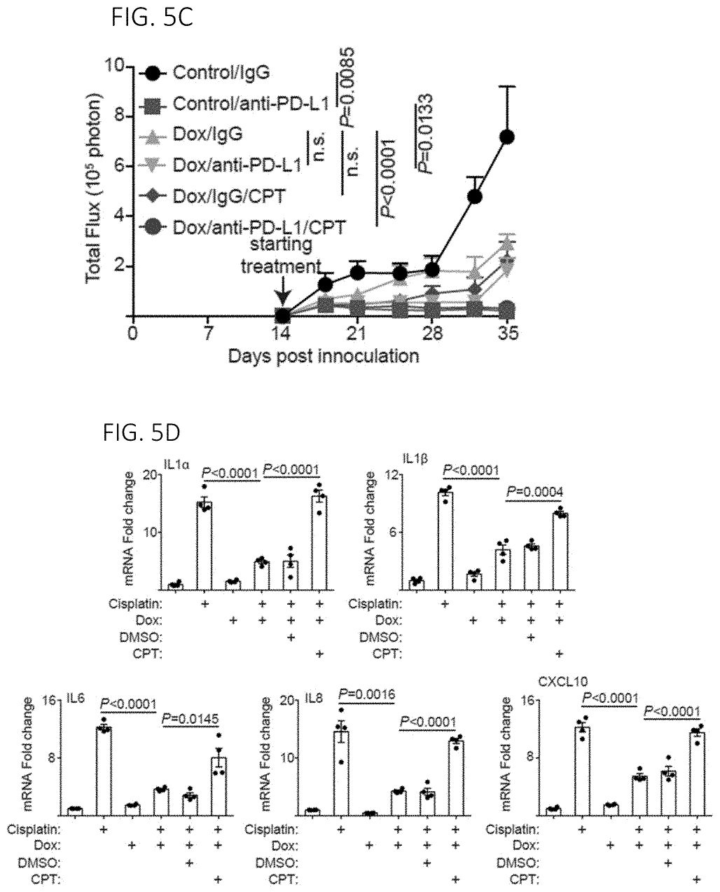

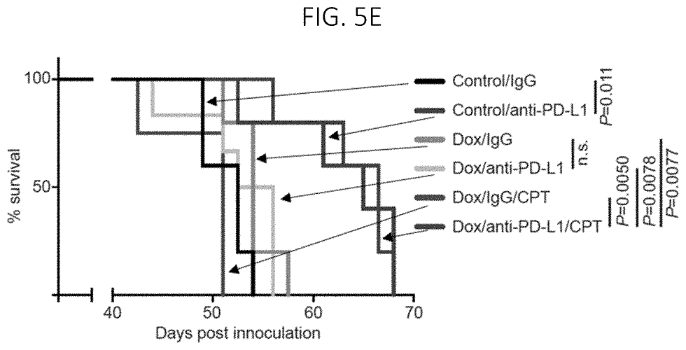

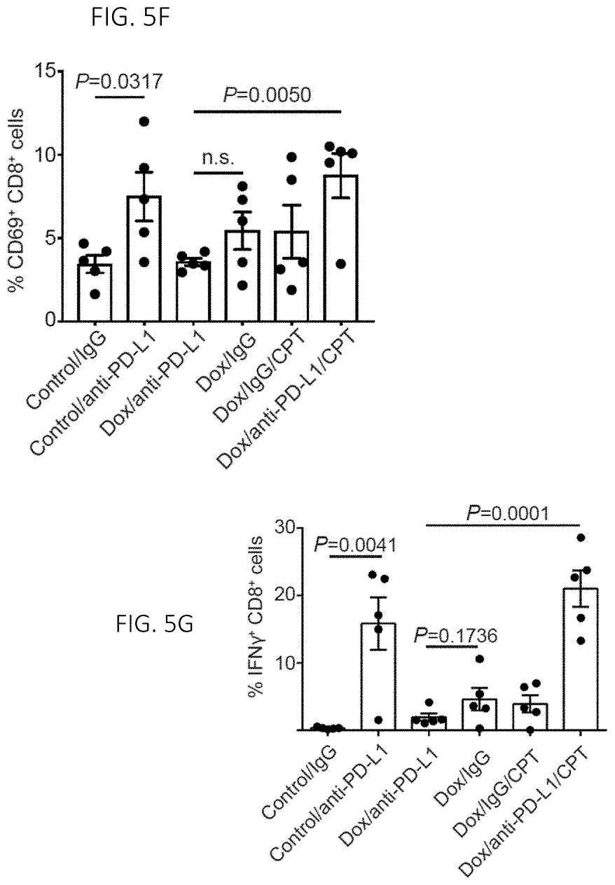

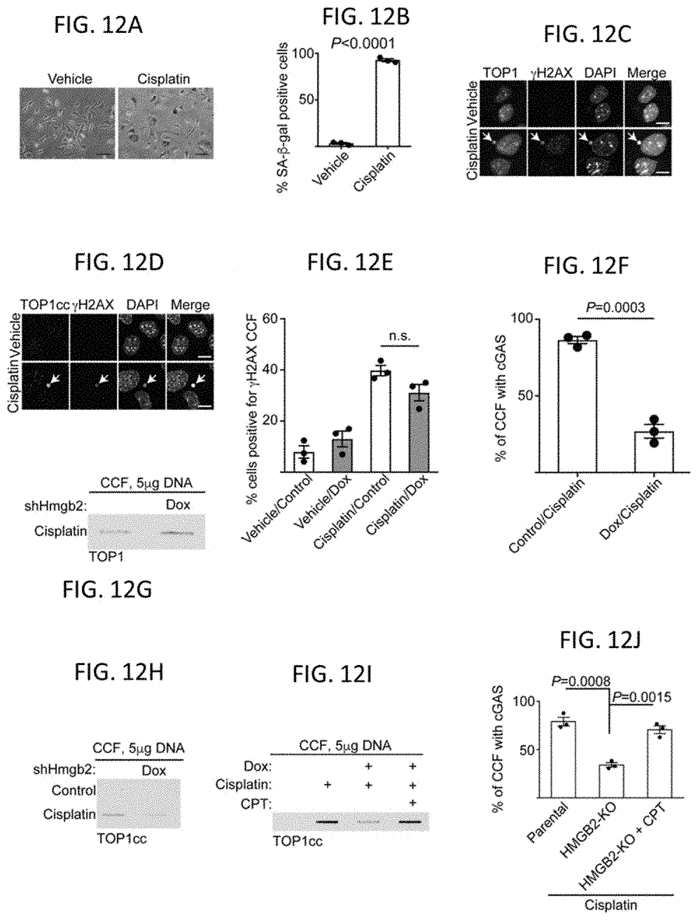

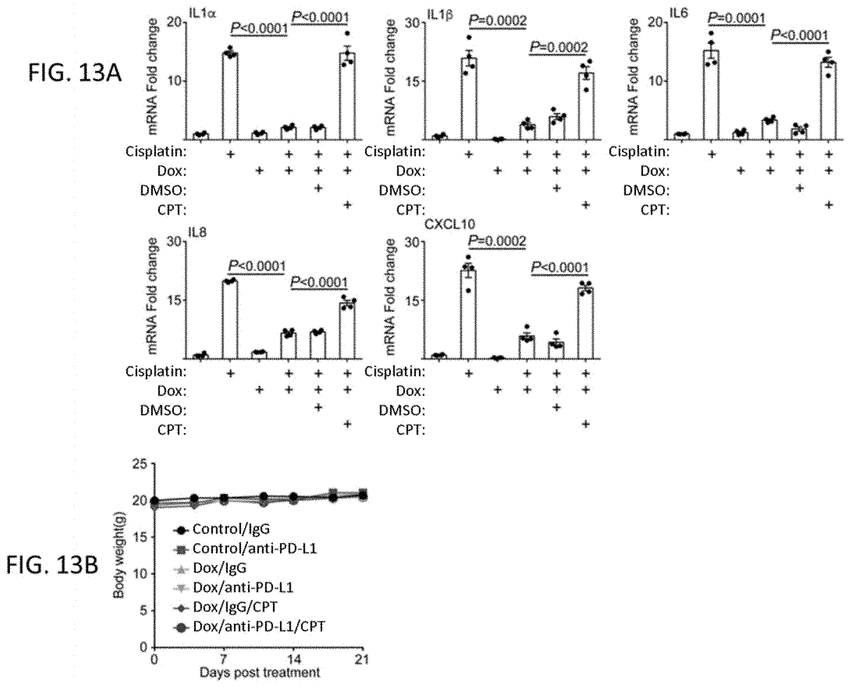

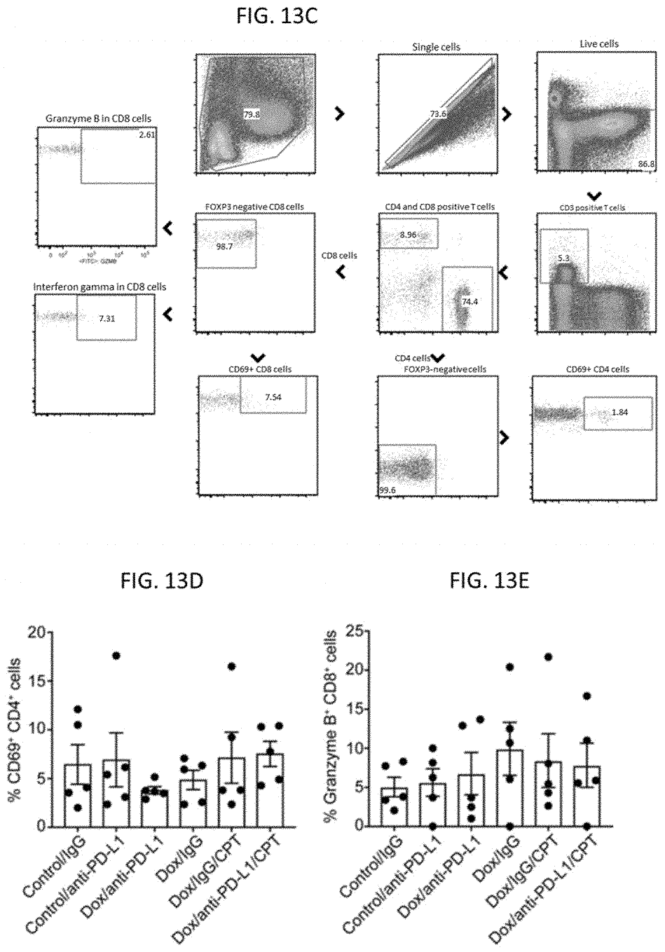

There is evidence to support that cGAS and its mediated expression of immune modulatory molecules such as SASP factors are essential for the antitumor effect of immune checkpoint blockade such as anti-PD-L1 antibody treatment (Xiang et al., 2017). To examine the relevance of the HMGB2-TOP1cc-cGAS pathway in immune checkpoint blockade treatment, we utilized an immune competent syngeneic ovarian cancer ID8-Defb29 Vegf-a mouse model (Zhu et al., 2016; Conejo-Garcia et al., 2004). Notably, the HMGB2-cGAS-TOP1cc axis is conserved in cisplatin-induced senescent ID8-Defb29/Vegf-a cells and cisplatin induced senescence in nearly 100% of the treated cells (FIG. 12A-12J). To examine the effects of HMGB2 loss during senescence on the response to the anti-PD-L1 antibody treatment, we treated ID8-Defb29 Vegf-a cells with cisplatin to induce senescence ex vivo with or without inducible HMGB2 knockdown as previously shown for radiation-induced cGAS-mediated inflammatory response (Harding et al., 2017). Then, we orthotopically transplanted the senescent cells into C57BL/6 mice by i.p. injection. Two weeks after transplantation, we randomized mice into different treatment groups. Compared with control tumors treated with or without anti-PD-L1 antibody, HMGB2 knockdown significantly abrogated the response to anti-PD-L1 antibody treatment (FIG. 5A-5C). This correlated with suppression of the expression of SASP genes both in vitro and in vivo in the sorted orthotopically transplanted tumor cells (FIG. 5D and FIG. 13A). Since HMGB2 is required for cGAS-dependent activation of SASP genes, these results are consistent with the literature that cGAS and its regulated immune modulating molecules such as SASP are essential for the antitumor effect of immune checkpoint blockade (Xiang et al., 2017). Since CPT treatment rescues recognition of CCF by cGAS and SASP when HMGB2 is inhibited (FIG. 2E-2I), we treated the HMGB2 knockdown tumors with CPT to determine whether CPT treatment is sufficient to restore the anti-PD-L1 treatment response in these tumors. Indeed, CPT treatment significantly restored the anti-PD-L1 response (FIG. 5B-5C). Consistently, compared with control tumors treated with anti-PD-L1, HMGB2 knockdown erased the survival advantage improved by anti-PD-L1 antibody treatment (FIG. 5E). Notably, CPT treatment rescued the survival of mice bearing HMGB2 knockdown tumors to a degree that is comparable to mice bearing control tumors treated with an anti-PD-L1 antibody (FIG. 5E). However, CPT treatment did not affect the body weight of the treated mice (FIG. 13B), suggesting that CPT did not exhibit toxicity in anti-PD-L1 antibody-treated group. Consistent with a requirement for T cell responses in the observed tumor suppressive effects by anti-PD-L1 blockade, both activated CD69⁺/CD8⁺ and IFNγ⁺/CD8⁺ T cells correlated with changes in survival in the different treatment groups (FIGS. 5F-G and FIG. 11C). Notably, the activated CD69⁺/CD4⁺ or Granzyme B⁺/CD8 T cells were not changed among the different treatment groups (FIG. 11D-11E). Together, we conclude that the status of the HMGB2-TOP1cc-cGAS axis determines the response to immune checkpoint blockade.

### Discussion

Consistent with previous reports (Aird et al., 2016), HMGB2 knockdown suppresses the growth of the tumor cells (FIG. 5C). However, HMGB2 expression is critical for response to checkpoint blockade in the context of therapy-induced senescence. This is due to its role in mediating SASP that is important for checkpoint blockade response. Thus, the role of HMGB2 in therapy response is context dependent. In addition, HMGB2 knockdown suppressed SASP and reduced the tumor growth in vivo (FIG. 5B-5C), which is consistent with the previous notion that SASP promotes tumor growth in a context dependent manner (Rodier et al., 2011). HMGB2 knockdown or knockout increased TOP1 levels in CCF (FIG. 4E). However, this was not sufficient to compensate for the decrease in TOP1cc levels in CCF. Thus, the lack of TOP1cc due to its destabilization contributes to suppression of SASP by HMGB2 inhibition. This also explains the increase in TOP1 and a decrease in TOP1cc levels in CCF of HMGB2 knockdown or knockout senescent cells (FIG. 4E). Thus, our findings identified a critical component in the cGAS-mediated inflammation response by providing a molecular mechanism through which cytoplasmic chromatin is recognized by cGAS.

Here we identified the TOP1cc, a TOP1 covalently modified DNA complex, as a critical mediator of the recognition of CCF by cGAS through direct interaction between TOP1 and cGAS in a dsDNA dependent manner during senescence. In addition, we showed that HMGB2 functions upstream of the TOP1cc-cGAS axis by stabilizing TOP1cc. Thus, our studies provided additional mechanistic insights into how HMGB proteins boost cytosolic nucleic-acid sensing (Yanai et al., 2009). Finally, we show that the HMGB2-TOP1cc-cGAS axis functionally regulates SASP and the response to immune checkpoint blockade. These findings indicate that clinically applicable TOP1 inhibitors such as CPT can serve as a sensitizer to immune checkpoint blockade to target therapy-induced senescent cells. Notably, TOP1 inhibitors increase the sensitivity of patient-derived melanoma cell lines to T-cell-mediated cytotoxicity (McKenzie et al., 2018; Haggerty et al., 2011). This is consistent with our findings that TOP1 inhibitors-induced TOP1cc boosts cGAS-mediated inflammation and the associated immune checkpoint blockade treatment.

### Example 3: Sensitization of Ovarian Tumor to Immune Checkpoint Blockade by Boosting Senescence-Associated Secretory Phenotype

Therapy-induced senescence-associated secretory phenotype (SASP) correlates with overcoming resistance to immune checkpoint blockade (ICB). Intrinsic resistance to ICB is a major clinical challenge. For example, ovarian cancer is largely resistant to ICB. Here we show that adoptive transfer of SASP-boosted ex vivo therapy-induced senescent cells sensitizes ovarian tumors to ICB. Topoisomerase 1 (TOP1) inhibitors such as irinotecan enhance cisplatin-induced SASP, which depends on the TOP1 cleavage complex-regulated cGAS pathway. Transfer of cisplatin-induced, SASP-boosted senescent cells with irinotecan sensitizes ovarian tumor to anti-PD-1 antibody and improves the survival of tumor-bearing mice in an immunocompetent, syngeneic model. This correlates with the infiltration of transferred senescent cells in the established orthotopic tumors and an increase in the infiltration of activated CD8+ T cells and dendritic cells in the tumor bed.

### Results

### Isolation of SASP-Boosted, Therapy-Induced Senescent Ovarian Cancer Cells

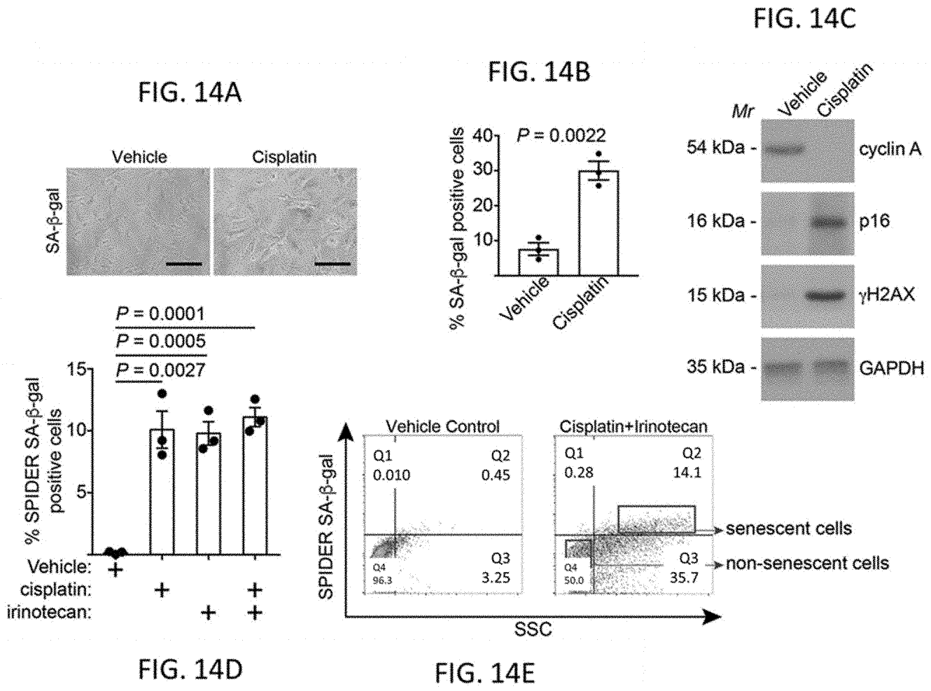

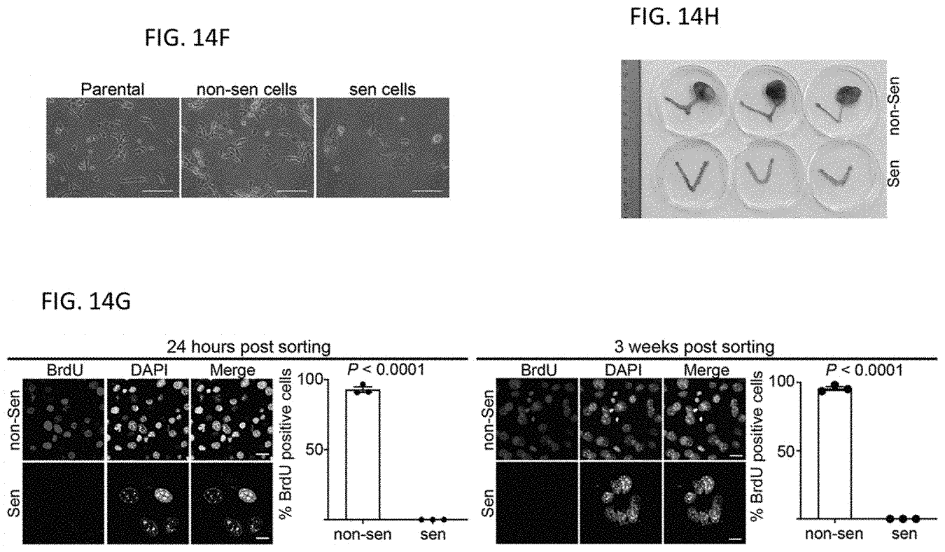

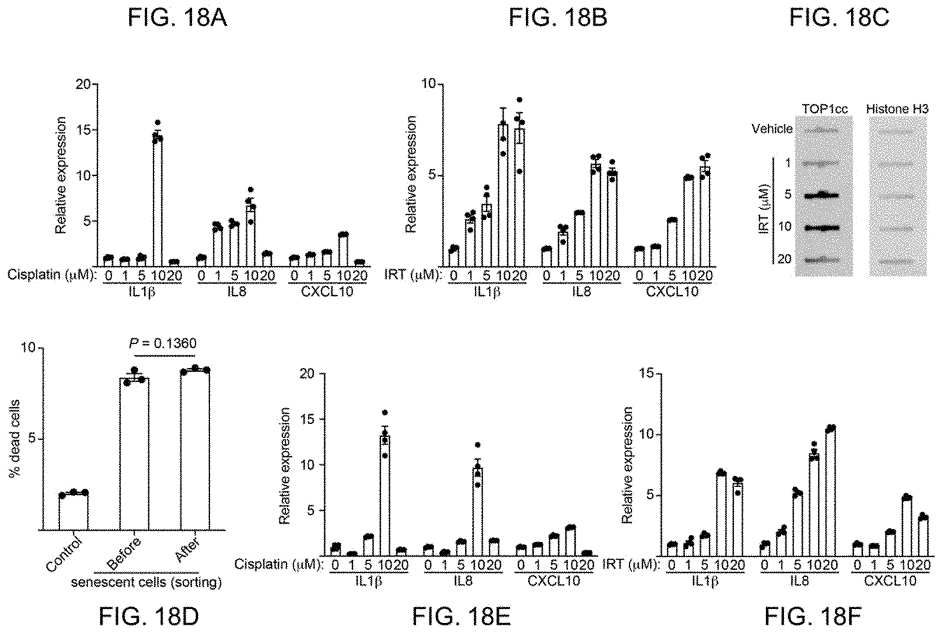

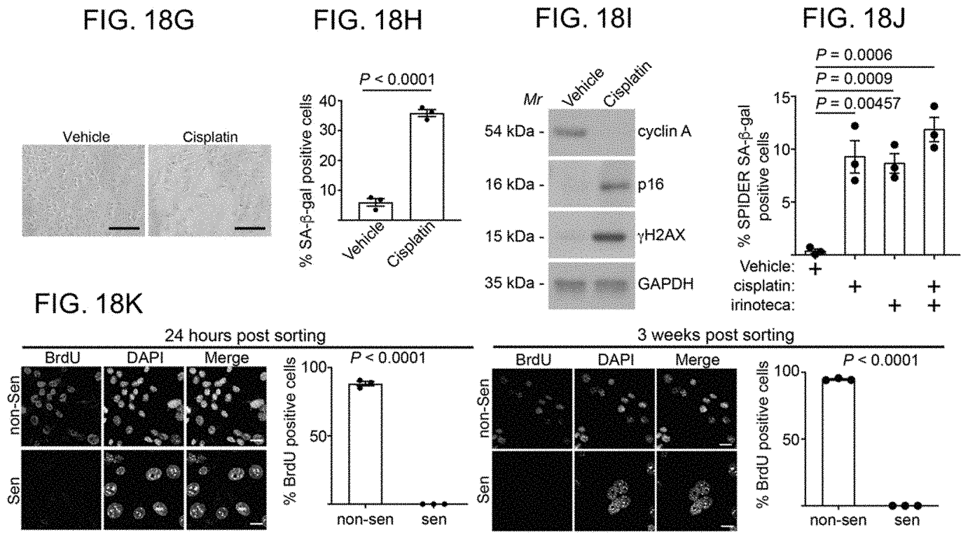

To isolate senescent cells for adoptive transfer, we treated UPK10 mouse ovarian cancer cells with cisplatin to induce senescence as evidenced by induction of markers of senescence including senescence-associated β-galactosidase (SA-β-Gal) activity, p16 and γH2AX (FIG. 14A-FIG. 14C). This was accompanied by a decrease in cell proliferation marker cyclin A (FIG. 14C). UPK10 cells were isolated from mouse ovarian tumors developed from conditional activation of Kras and inactivation of Tp53 that fully recapitulated the immune microenvironment of human ovarian cancers (Scarlett et al., 2012). In addition, platinum-based chemotherapies such as cisplatin are standard of care for ovarian cancer (Lheureux et al., 2019). We chose 10 μM cisplatin based on optimal induction of SASP factors such as IL1β, IL8, and CXCL10 in a dose-titration study (FIG. 18A). Since TOP1 inhibitors enhance SASP without affecting senescence-associated growth arrest, we combined cisplatin and a clinically applicable TOP1 inhibitor irinotecan (Pommier et al., 2016). The dose of irinotecan was determined based on optimal induction of SASP factors such as IL1β, IL8, and CXCL10 as well as TOP1cc in a dose-titration study (FIG. 18B and FIG. 18C). Notably, the percentage of senescent cells induced by cisplatin with or without irinotecan was comparable as determined by a fluorescence-based marker of senescence, SPiDER SA-β-Gal activity (FIG. 14D). Interestingly, irinotecan alone also induced SA-β-Gal activity, which is consistent with the notion that activation of TOP1cc-regulated cGAS pathway induces senescence and SASP (Yang et al., 2017). Next, we sorted senescent cells induced by a combination of cisplatin and irinotecan using flow cytometry based on expression of fluorescence SPiDER SA-β-Gal activity and larger sizes of senescent cells (FIG. 14E and FIG. 14F). Notably, flow cytometry sorting did not significantly stress the senescent cells to increase cell death (FIG. 18D). Validating our senescent cells sorting strategy, cell proliferation markers such as BrdU incorporation was negative in re-cultured, sorted senescent cells compared with non-senescent cells even after three weeks of culture (FIG. 14G). Similar results were also obtained in ID8 mouse ovarian cancer cells (FIG. 18E-FIG. 18K), indicating that this is not a cell line specific effect. Finally, to examine the growth potential of the sorted senescent cells in vivo, we orthotopically transplanted the sorted senescent cells into mouse bursa that covers the mouse ovary to mimic the in vivo tumor microenvironment. Notably, sorted control non-senescent cells formed tumors that reached ethical limit in one month. In contrast, sorted senescent cells that were orthotopically transplanted in parallel failed to form visible tumors in two and half months (FIG. 14F). Together, we concluded that it is feasible to sort out growth-arrested, therapy-induced senescent cells in vitro.

TOP1 Inhibitor Irinotecan Boosts SASP Through the cGAS Pathway

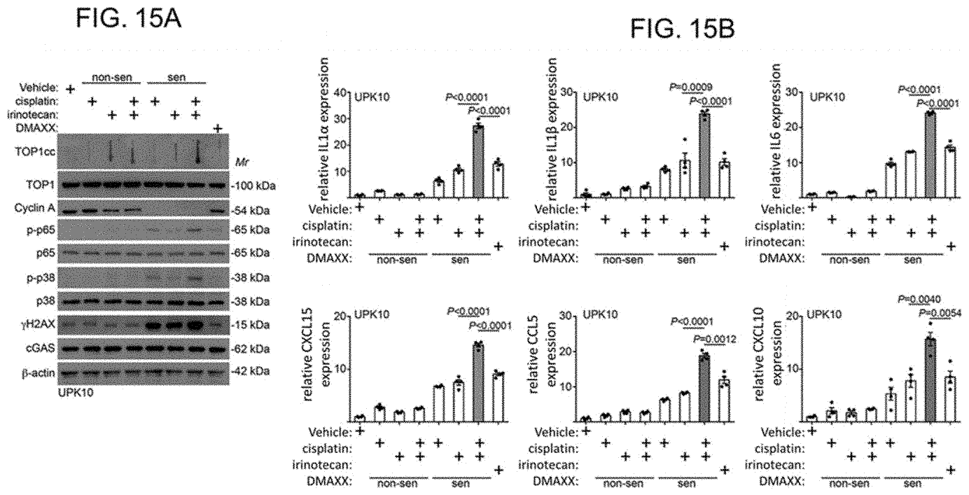

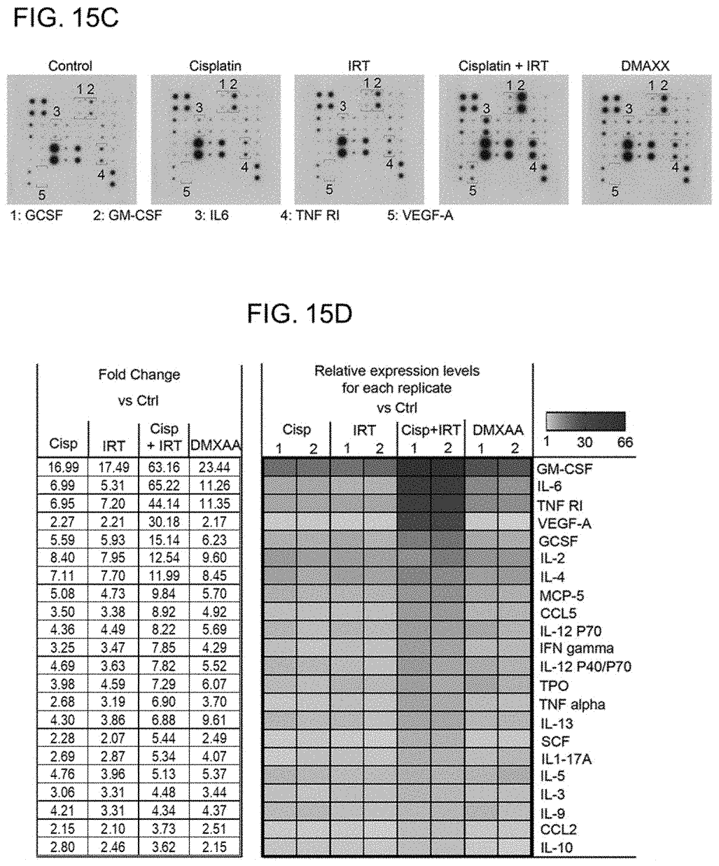

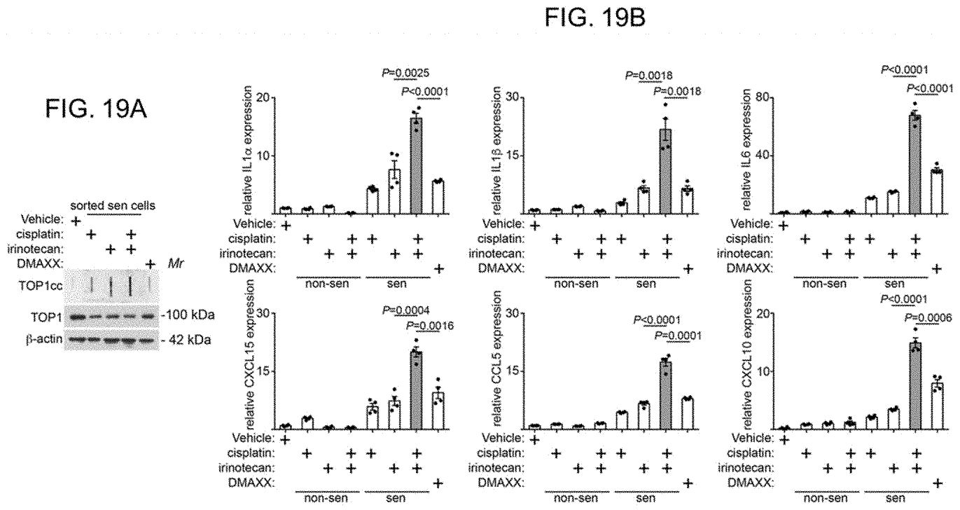

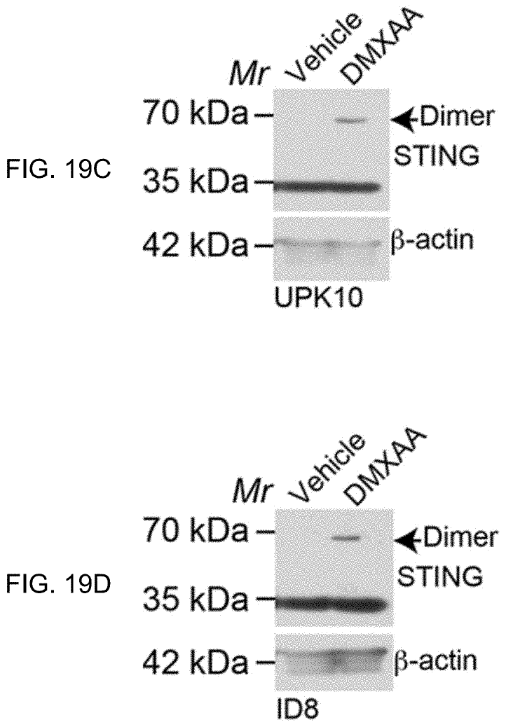

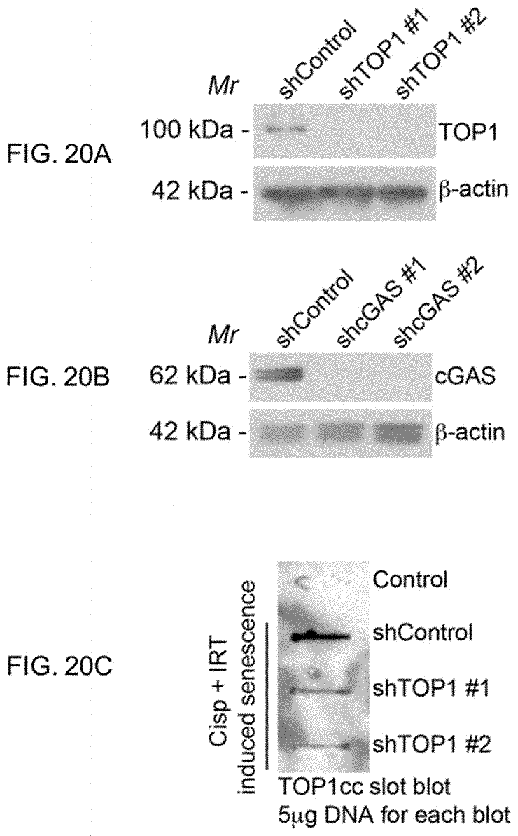

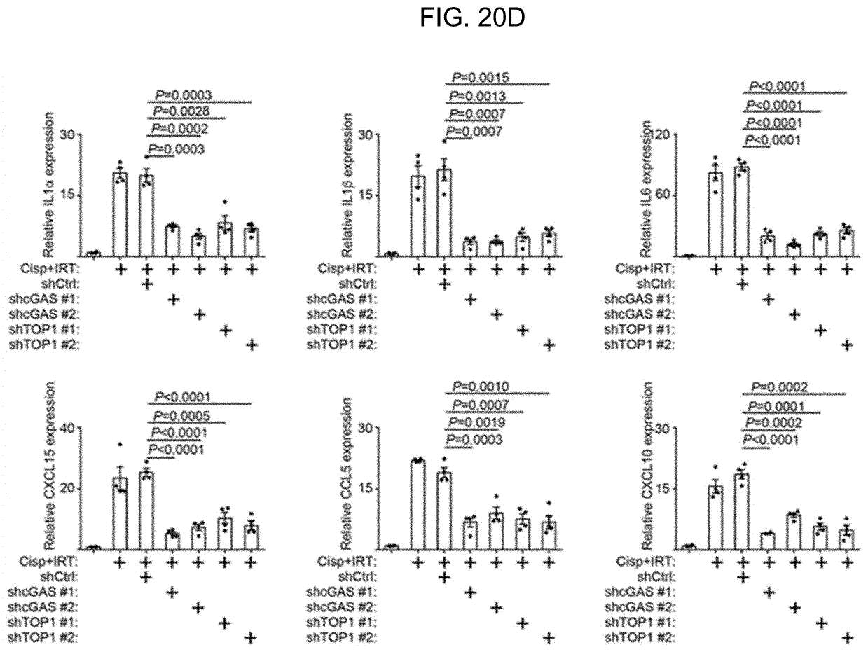

We next sought to characterize the sorted senescent cells from the different treatment groups. Compared with cisplatin-induced senescent cells, TOP1cc levels were increased by irinotecan addition (FIG. 15A). Interestingly, TOP1cc levels were notably higher in the sorted non-senescent cells treated with irinotecan or a combination compared with vehicle control treated non-senescent cells (FIG. 15A). However, these cells are not senescent as evidenced by expression of cell proliferation markers such as cyclin A (FIG. 15A). This suggests that TOP Ice alone is not sufficient to induce senescence. We next examined changes in expression of SASP factors by quantitative reverse transcription polymerase chain reaction (qRT-PCR) in the sorted non-senescent and senescent cells from the various treatment groups. Indeed, irinotecan significantly increased the expression of SASP factors induced by cisplatin at the mRNA levels (FIG. 15B), which correlated with an increase in SASP regulators such as phospho-p65 NF-κB and phosphor-p38 MAPK (FIG. 15A) (Herranz and Gil, 2018). Validating our sorting approach, the sorted non-senescent cells did not show overt increase in the expression of SASP factors (FIG. 15B). Similar findings were also made in ID8 mouse ovarian cancer cells (FIG. 19A and FIG. 19B). We further validated the increase in the secretion of SASP factors induced by irinotecan and cisplatin combination using an antibody array (FIG. 15C and FIG. 15D). As a control, DMAXX, an STING agonist in mouse cells (Conlon et al., 2013), is sufficient to increase the expression and secretion of SASP factors, albeit at a significantly lower levels compared with those in the senescent cells sorted from cisplatin and irinotecan combination treatment (FIG. 15B-FIG. 15D, FIG. 19C, and FIG. 19D). Together, we concluded that TOP1 inhibitor irinotecan boosts SASP in the senescent cells induced by cisplatin.

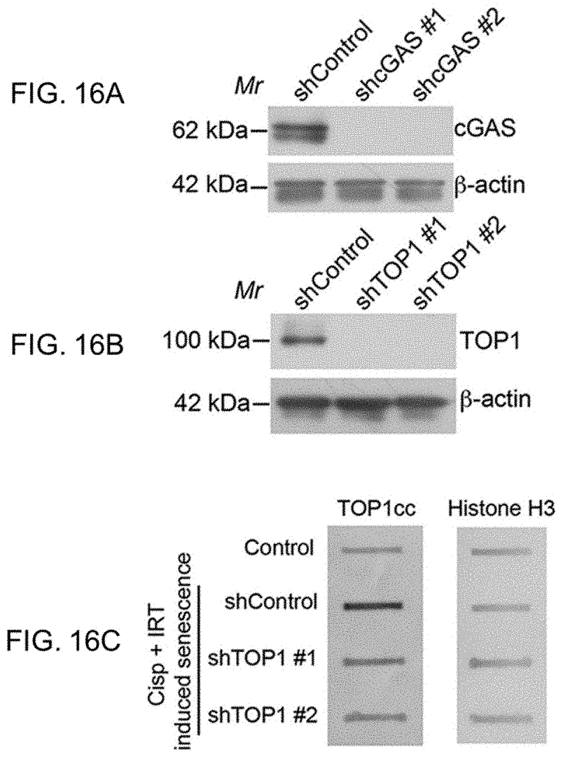

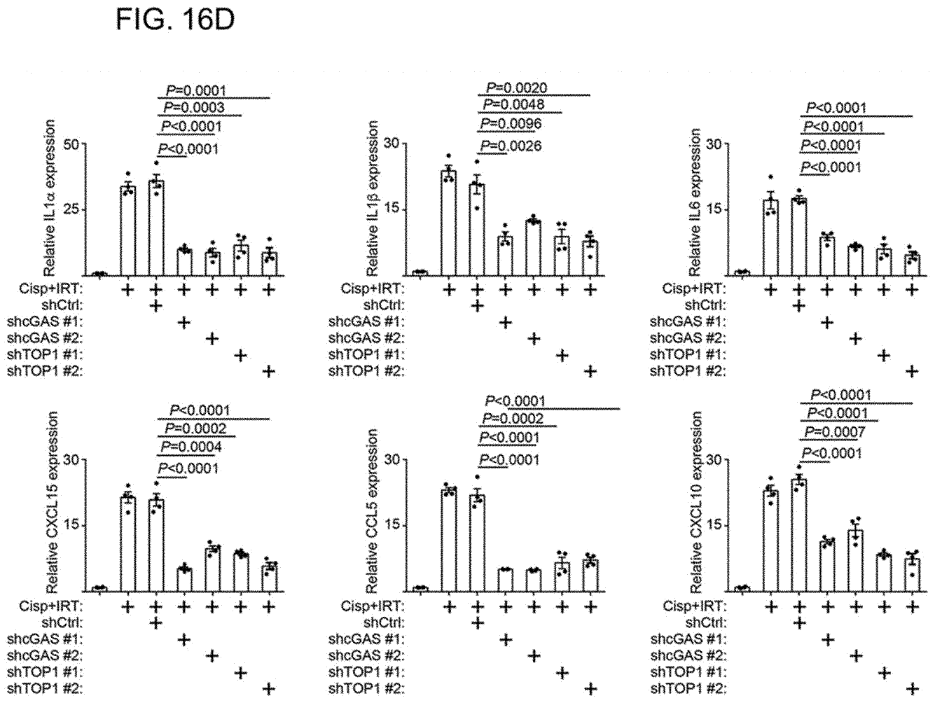

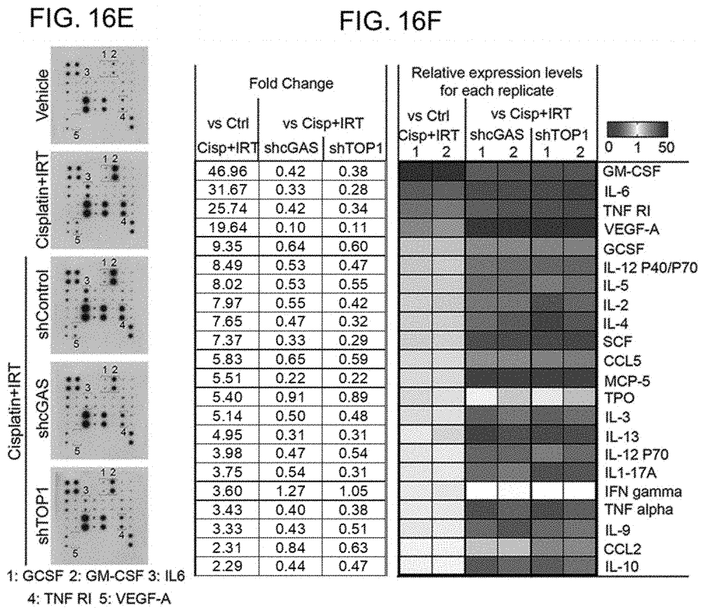

We next sought to determine whether the observed enhancement of SASP by irinotecan is TOP1 and cGAS dependent. Toward this goal, we knocked down TOP1 or cGAS using two independent shRNAs to limit potential off-target effects (FIG. 16A and FIG. 16B). Consistently, TOP1 knockdown decreased TOP1cc levels induced by irinotecan and cisplatin combination (FIG. 3C). Indeed, knockdown of either TOP1 or cGAS significantly suppressed the expression of SASP genes as determined by qRT-PCR (FIG. 16D). Consistently, secretion of SASP factors was also significantly decreased by knockdown of either cGAS or TOP1 in the sorted senescent cells induced by cisplatin and irinotecan combination (FIG. 16E and FIG. 16F). Together, these findings support the notion that the observed enhancement of SASP by irinotecan in cisplatin-induced senescent cells was mediated by TOP1cc-regulated cGAS pathway.

### Transfer of SASP-Boosted Senescent Cells Sensitizes Ovarian Tumor to Anti-PD-1 Antibody

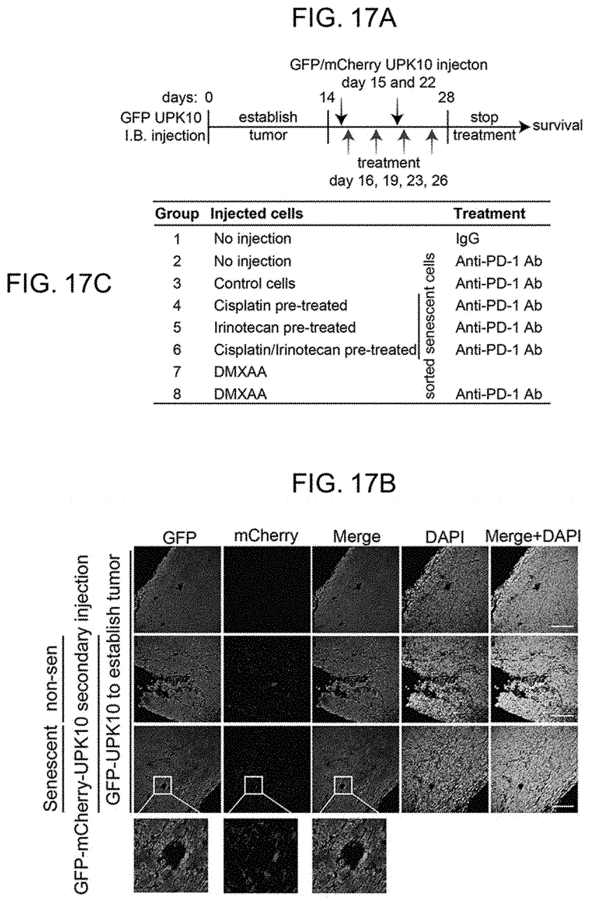

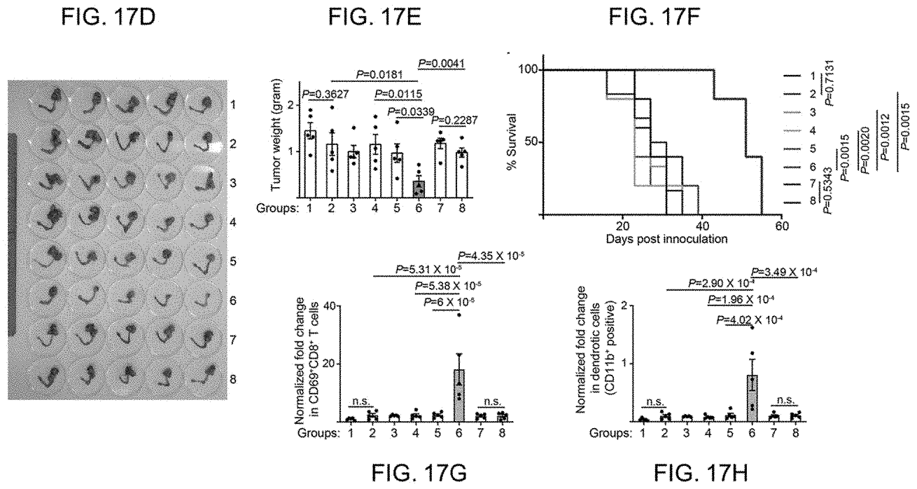

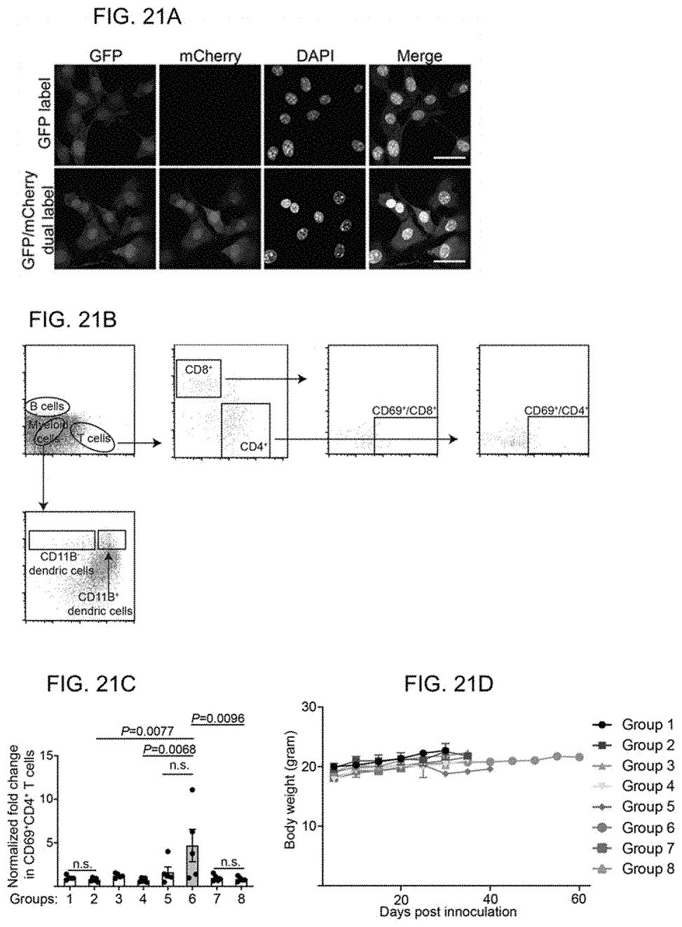

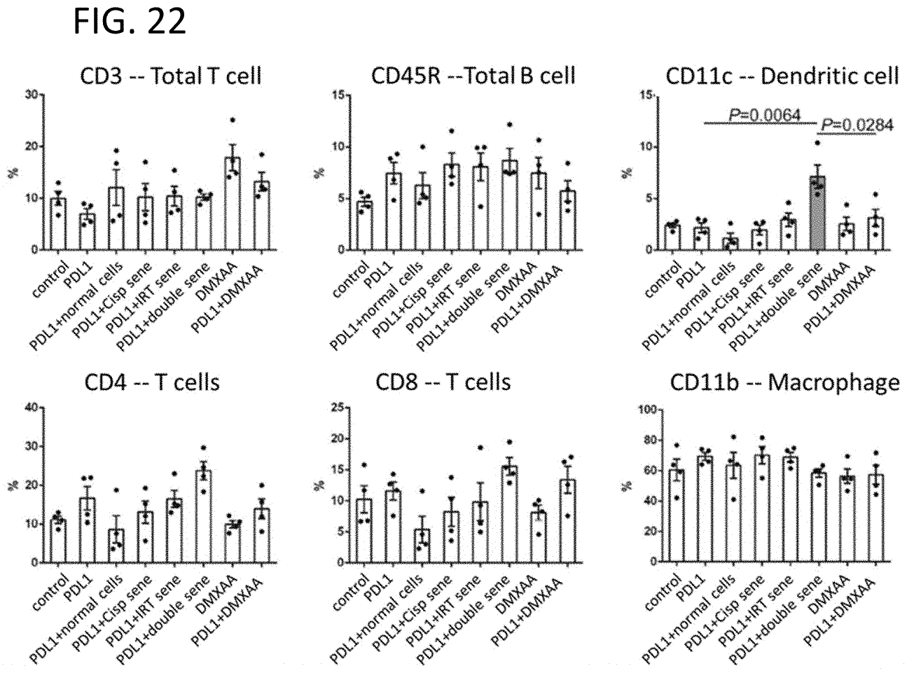

Given the critical role played by cGAS in mediating ICB (Xiang et al., 2017) and the evidence that induction of inflammatory SASP correlates with sensitization of resistant melanomas to ICB (Jerby-Amon et al., 2018), we sought to explore the possibility of adoptive transfer of SASP-boosted senescent cells as a potential cell therapy to sensitize tumors to ICB. Toward this goal, we established a syngeneic, immunocompetent mouse ovarian tumor model using UPK10 cells (Scarlett et al., 2012). We orthotopically transplanted UPK10 into the mouse bursa and allowed the tumor to establish for two weeks (FIG. 17A). We transplanted sorted control non-senescent or senescent UPK10 cells induced ex vivo by cisplatin, irinotecan or a combination by i.p. injection on day 15 and 22 and followed with anti-PD-1 antibody treatment on day 16, 19, 23, and 26 (FIG. 17A). To differentiate the pre-established tumors formed by GFP-positive UPK10 cells from those of i.p. injected UPK10 cells, we labeled the subsequently injected sorted control non-senescent and senescent cells with mCherry that are GFP and mCherry double positive (FIG. 21A). Notably, both non-senescent and senescent mCherry positive cells infiltrated the pre-established GFP-positive orthotopic tumors formed by GFP-positive UPK10 cells (FIG. 17B). This result suggests that the adoptively transferred, SASP-boosted senescent ovarian cancer cells are capable of infiltrating the pre-existing tumor sites. Notably, anti-PD-1 antibody was not effective against the pre-established UPK10 tumors compared with IgG controls (FIG. 17C-FIG. 17E). Interestingly, senescent cells sorted from the cisplatin or irinotecan treatment alone did not significantly reduce tumor burden in response to anti-PD-1 antibody treatment (FIG. 17C-FIG. 17E). However, the injection of sorted SASP-boosted senescent cells induced by a combination of cisplatin and irinotecan significantly reduced the tumor burden as indicated by a reduction in tumor weight (FIG. 17C-FIG. 17E, group 6). Consistently, the survival of the tumor-bearing mice in this group was significantly improved (FIG. 17F). Notably, the injection of sorted non-senescent control cells did not increase tumor growth (FIG. 17C-FIG. 17E). This might be caused by partial effects of anti-PD-1 antibody treatment in this group or a masking effect caused by the growth of the pre-established tumors. FIG. 22 shows the percentage of the indicated immune cells in the tumor samples. Consistent with previous reports that SASP-accompanied sensitization of ICB is mediated by CD8+ T cell (Jerby-Amon et al., 2018), we observed an increase in infiltrated activated CD69*/CD8+ T cells in the tumor bed in group 6 (FIG. 17G, FIG. 17H, and FIG. 21B). In addition, we observed an increase in CD11b⁺ dendritic cells in group 6 compared with other groups (FIG. 17G and FIG. 17H). There was an increase in infiltration of activated CD69⁺/CD4⁺ T cells in group 6 compared with group 4, but not group 5 (FIG. 21C). Notably, transfer of DMXAA ex vivo treated cells did not affect the response to anti-PD-1 and failed to reduce tumor burden or improve the survival of tumor-bearing mice (FIG. 17C-FIG. 17F). Consistently, neither CD69⁺/CD8⁺ T cells nor CD11b⁺ dendritic cells were significantly affected by the transfer of DMXAA ex vivo treated cells (FIG. 17G and FIG. 17H). Notably, no overt toxicity associated with adoptive transfer of SASP-boosted, cisplatin-induced senescent ovarian cancer cells was observed. For example, the body weight of tumor-bearing mice was not significantly reduced compared with other treatment groups (FIG. 21D). Together we conclude that adoptive transfer of SASP-boosted cisplatin-induced senescent ovarian cancer cells using TOP1 inhibitor irinotecan sensitizes ovarian tumors to ICBs.

### Discussion

Despite the fact that SASP-promoting cGAS is required for response to ICB (Xiang et al., 2017) and therapy-induced SASP correlates with overcoming resistance to ICBs (Jerby-Amon et al., 2018; Ruscetti et al., 2020), therapeutic approaches that leverage SASP of senescent cells to sensitize tumors to ICB have not been reported. Here we show that adoptive transfer of SASP-boosted, cisplatin-induced senescent cells using clinically applicable TOP1 inhibitor irinotecan sensitizes ovarian tumor to ICB. An advantage of this approach is that the treatment occurs ex vivo, which limits the potential systematic toxicity caused by direct treatment with these small molecules in vivo. Consistent with our findings, TOP1 inhibitors increase the sensitivity of patient-derived melanoma cell lines to T-cell-mediated cytotoxicity (Haggerty et al., 2011; McKenzie et al., 2018).

Notably, the observed sensitization correlates with infiltration of senescent cells into the tumor bed. Indeed, previous studies show that intravenously or subcutaneously injected ovarian cancer cells metastasize to ovary (Bai et al., 2019). This raised the possibility that transfer of SASP-boosted senescent cells may convert “cold” into “hot” tumors through infiltration of senescent cells into tumor bed and associated secretion of inflammatory SASP factors. However, we cannot exclude the possibility that the transferred senescent cells may localize to other areas. In addition, further studies are warranted to elucidate what SASP factors mediate the observed antitumor response and what cells are being impacted to regulate therapy response. Further, this approach in combination with subsequent ICB treatment allows for targeting and eradicating residual tumor nodules to prevent relapse, a major challenge in clinical management of ovarian cancer.

Although cisplatin-induced senescent cells are positive for SASP, their adoptive transfer was not sufficient to sensitize tumors to ICB. This supports the notion that levels of SASP dictate the outcome of adoptively transferred senescent cells. Consistently, STING agonist alone stimulated the expression of the SASP factors to a level that is comparable to those observed in cisplatin-induced senescent cells. However, this is not sufficient to sensitize tumors to ICB. Notably, UPK10 cells were isolated from mouse ovarian tumors developed from conditional activation of Kras and inactivation of Tp53 (Scarlett et al., 2012). In contrast, ID8 is wild-type for both Kras and Tp53. Given the fact that irinotecan boosted SASP induced by cisplatin in both UPK10 and ID8 cells, these findings suggest that the observed effects are independent of Kras or Tp53 status.

### REFERENCES

Aird, K. M. et al. HMGB2 orchestrates the chromatin landscape of senescence-associated secretory phenotype gene loci. The Journal of cell biology 215, 325-334, doi:10.1083/jcb.201608026 (2016).
Andreeva, L. et al. cGAS senses long and HMGB/TFAM-bound U-turn DNA by forming protein-DNA ladders. Nature 549, 394-398, doi:10.1038/nature23890 (2017).
Bai, S., Zhu, W., Coffman, L., Vlad, A., Schwartz, L. E., Elishaev, E., Drapkin, R., and Buckanovich, R. J. (2019). CD105 Is Expressed in Ovarian Cancer Precursor Lesions and is Required for Metastasis to the Ovary. Cancers (Basel) 11.
Barber, G. N. STING: infection, inflammation and cancer. Nat Rev Immunol 15, 760-770, doi:10.1038/nri3921 (2015).
Campisi, J. Aging, cellular senescence, and cancer. Annu Rev Physiol 75, 685-705, doi:10.1146/annurev-physiol-030212-183653 (2013).
Conejo-Garcia, J. R. et al. Tumor-infiltrating dendritic cell precursors recruited by a beta-defensin contribute to vasculogenesis under the influence of Vegf-A. Nat Med 10, 950-958, doi:10.1038/nm1097 (2004).
Conlon, J., Burdette, D. L., Sharma, S., Bhat, N., Thompson, M., Jiang, Z., Rathinam, V. A., Monks, B., Jin, T., Xiao, T. S., et al. (2013). Mouse, but not human STING, binds and signals in response to the vascular disrupting agent 5,6-dimethylxanthenone-4-acetic acid. J Immunol 190, 5216-5225.
Coppe, J. P., Patil, C. K., Rodier, F., Sun, Y., Munoz, D. P., Goldstein, J., Nelson, P. S., Desprez, P. Y., and Campisi, J. (2008). Senescence-associated secretory phenotypes reveal cell nonautonomous functions of oncogenic RAS and the p53 tumor suppressor. PLoS Biol 6, 2853-2868.
Cox, J. & Mann, M. MaxQuant enables high peptide identification rates, individualized p.p.b.-range mass accuracies and proteome-wide protein quantification. Nat Biotechnol 26, 1367-1372, doi:10.1038/nbt.1511 (2008).
Darvin, P., Toor, S. M., Sasidharan Nair, V., and Elkord, E. (2018). Immune checkpoint inhibitors: recent progress and potential biomarkers. Exp Mol Med 50, 1-11.
Dou, Z., Ghosh, K., Vizioli, M. G., Zhu, J., Sen, P., Wangensteen, K. J., Simithy, J., Lan, Y., Lin, Y., Zhou, Z., et al. (2017). Cytoplasmic chromatin triggers inflammation in senescence and cancer. Nature 550, 402-406.
Gluck, S., Guey, B., Gulen, M. F., Wolter, K., Kang, T. W., Schmacke, N. A., Bridgeman, A., Rehwinkel, J., Zender, L., and Ablasser, A. (2017). Innate immune sensing of cytosolic chromatin fragments through cGAS promotes senescence. Nat Cell Biol 19, 1061-1070.
Haggerty, T. J., Dunn, I. S., Rose, L. B., Newton, E. E., Martin, S., Riley, J. L., and Kurnick, J. T.(2011). Topoisomerase inhibitors modulate expression of melanocytic antigens and enhance T cell recognition of tumor cells. Cancer Immunol Immunother 60, 133-144.
Harding, S. M. et al. Mitotic progression following DNA damage enables pattern recognition within micronuclei. Nature 548, 466-470, doi:10.1038/nature23470 (2017).
Herranz, N., and Gil, J. (2018). Mechanisms and functions of cellular senescence. J Clin Invest 128, 1238-1246.
Ivanov, A. et al. Lysosome-mediated processing of chromatin in senescence. The Journal of cell biology 202, 129-143, doi:10.1083/jcb.201212110 (2013).
Jerby-Arnon, L., Shah, P., Cuoco, M. S., Rodman, C., Su, M. J., Melms, J. C., Leeson, R., Kanodia, A., Mei, S., Lin, J. R., et al. (2018). A Cancer Cell Program Promotes T Cell Exclusion and Resistance to Checkpoint Blockade. Cell 175, 984-997 e924.
Lheureux, S., Braunstein, M., and Oza, A. M. (2019). Epithelial ovarian cancer: Evolution of management in the era of precision medicine. CA Cancer J Clin 69, 280-304.
Li, X. et al. Cyclic GMP-AMP synthase is activated by double-stranded DNA-induced oligomerization. Immunity 39, 1019-1031, doi:10.1016/j.immuni.2013.10.019 (2013).
Liu, Z. S. et al. G3BP1 promotes DNA binding and activation of cGAS. Nature immunology 20, 18-28, doi:10.1038/s41590-018-0262-4 (2019).
Ly, P. et al. Selective Y centromere inactivation triggers chromosome shattering in micronuclei and repair by non-homologous end joining. Nat Cell Biol 19, 68-75, doi:10.1038/ncb3450 (2017).
Mackenzie, K. J. et al. cGAS surveillance of micronuclei links genome instability to innate immunity. Nature 548, 461-465, doi:10.1038/nature23449 (2017).
McKenzie, J. A., Mbofung, R. M., Malu, S., Zhang, M., Ashkin, E., Devi, S., Williams, L., Tieu, T., Peng, W., Pradeep, S., et al. (2018). The Effect of Topoisomerase I Inhibitors on the Efficacy of T-Cell-Based Cancer Immunotherapy. J Natl Cancer Inst 110, 777-786.
Meng et al, Radiation-inducible Immunotherapy for Cancer: Senescent Tumor Cells as a Cancer Vaccine Molecular Therapy, 20(5):1046-1055, May 2012.
Nacarelli, T. et al. NAD(+) metabolism governs the proinflammatory senescence-associated secretome. Nat Cell Biol 21, 397-407, doi:10.1038/s41556-019-0287-4 (2019).
O'Donnell, J. S., Long, G. V., Scolyer, R. A., Teng, M. W., and Smyth, M. J. (2017). Resistance to PD1/PDL1 checkpoint inhibition. Cancer Treat Rev 52, 71-81.
Pommier, Y., Sun, Y., Huang, S. N., and Nitiss, J. L. (2016). Roles of eukaryotic topoisomerases in transcription, replication and genomic stability. Nat Rev Mol Cell Biol 17, 703-721.
Rodier, F. & Campisi, J. Four faces of cellular senescence. J Cell Biol 192, 547-556, doi:10.1083/jcb.201009094 (2011).
Ruscetti, M., Morris, J. P. t., Mezzadra, R., Russell, J., Leibold, J., Romesser, P. B., Simon, J., Kulick, A., Ho, Y. J., Fennell, M., et al. (2020). Senescence-Induced Vascular Remodeling Creates Therapeutic Vulnerabilities in Pancreas Cancer. Cell 181, 424-441 e421.
Sato, E., Olson, S. H., Ahn, J., Bundy, B., Nishikawa, H., Qian, F., Jungbluth, A. A., Frosina, D., Gnjatic, S., Ambrosone, C., et al. (2005). Intraepithelial CD8+ tumor-infiltrating lymphocytes and a high CD8+/regulatory T cell ratio are associated with favorable prognosis in ovarian cancer. Proc Natl Acad Sci USA 102, 18538-18543.
Scarlett, U.K., Rutkowski, M. R., Rauwerdink, A. M., Fields, J., Escovar-Fadul, X., Baird, J., Cubillos-Ruiz, J. R., Jacobs, A. C., Gonzalez, J. L., Weaver, J., et al. (2012). Ovarian cancer progression is controlled by phenotypic changes in dendritic cells. J Exp Med 209, 495-506.
Shimizu, N., Kanda, T. & Wahl, G. M. Selective capture of acentric fragments by micronuclei provides a rapid method for purifying extrachromosomally amplified DNA. Nature genetics 12, 65-71, doi:10.1038/ng0196-65 (1996).
Sun, L., Wu, J., Du, F., Chen, X. & Chen, Z. J. Cyclic GMP-AMP synthase is a cytosolic DNA sensor that activates the type I interferon pathway. Science 339, 786-791, doi: 10.1126/science.1232458 (2013).
Takahashi, A., Loo, T. M., Okada, R., Kamachi, F., Watanabe, Y., Wakita, M., Watanabe, S., Kawamoto, S., Miyata, K., Barber, G. N., et al. (2018). Downregulation of cytoplasmic DNases is implicated in cytoplasmic DNA accumulation and SASP in senescent cells. Nat Commun 9, 1249.
Thomas, J. O. & Travers, A. A. HMG1 and 2, and related ‘architectural’ DNA-binding proteins. Trends Biochem Sci 26, 167-174 (2001).
Thomas, J. O. HMG1 and 2: architectural DNA-binding proteins. Biochem Soc Trans 29, 395-401 (2001).
Wagner, V., and Gil, J. (2020). Senescence as a therapeutically relevant response to CDK4/6 inhibitors. Oncogene 39, 5165-5176.
Wang, W., Liu, J. R., and Zou, W. (2019). Immunotherapy in Ovarian Cancer. Surg Oncol Clin N Am 28, 447-464.
Wu, J. et al. Cyclic GMP-AMP is an endogenous second messenger in innate immune signaling by cytosolic DNA. Science 339, 826-830, doi:10.1126/science.1229963 (2013).
Xiang, Y., Laurent, B., Hsu, C. H., Nachtergaele, S., Lu, Z., Sheng, W., Xu, C., Chen, H., Ouyang, J., Wang, S., et al. (2017). RNA m(6)A methylation regulates the ultraviolet-induced DNA damage response. Nature 543, 573-576.
Xu, M., Pirtskhalava, T., Farr, J. N., Weigand, B. M., Palmer, A. K., Weivoda, M. M., Inman, C. L., Ogrodnik, M. B., Hachfeld, C. M., Fraser, D. G., et al. (2018). Senolytics improve physical function and increase lifespan in old age. Nat Med 24, 1246-1256.
Yanai, H. et al. HMGB proteins function as universal sentinels for nucleic-acid-mediated innate immune responses. Nature 462, 99-103, doi:10.1038/nature08512 (2009).
Yang, H., Wang, H., Ren, J., Chen, Q., and Chen, Z. J. (2017). cGAS is essential for cellular senescence. Proc Natl Acad Sci USA 114, E4612-E4620.
Zhang, L., Conejo-Garcia, J. R., Katsaros, D., Gimotty, P. A., Massobrio, M., Regnani, G., Makrigiannakis, A., Gray, H., Schlienger, K., Liebman, M. N., et al. (2003). Intratumoral T cells, recurrence, and survival in epithelial ovarian cancer. N Engl J Med 348, 203-213.
Zhu, H. et al. BET Bromodomain Inhibition Promotes Anti-tumor Immunity by Suppressing PD-L1 Expression. Cell Rep 16, 2829-2837, doi:10.1016/j.celrep.2016.08.032 (2016).

Each and every patent, patent application, and any document listed herein, and the sequence of any publicly available nucleic acid and/or peptide sequence cited throughout the disclosure, is/are expressly incorporated herein by reference in their entireties. U.S. Provisional Application No. 62/976,020, filed Feb. 13, 2020, is incorporated herein by reference in its entirety. Hao et al., Sensitization of ovarian tumor to immune checkpoint blockade by boosting senescence-associated secretory phenotype, iScience. 2020 Dec. 30; 24(1):102016 is incorporated by reference herein in its entirety. Embodiments and variations of this invention other than those specifically disclosed above may be devised by others skilled in the art without departing from the true spirit and scope of the invention. The appended claims include such embodiments and equivalent variations.

1. A method of treating cancer in a subject in need thereof, the method comprising administering therapy-induced senescent (TIS) cells and an immune checkpoint inhibitor to the subject.

2. The method according to claim 1, wherein the TIS cells are cancer cells which have been removed from the subject.

3. A method of treating cancer in a subject in need thereof, the method comprising
a) obtaining cancer cells from the subject;
b) treating the cancer cells with a chemotherapeutic agent or radiation and a TOP inhibitor, thereby inducing senescence;
c) optionally, confirming senescence and/or sorting senescent cells from non-senescent cells;
d) administering the senescent cells to the subject; and
e) administering a checkpoint inhibitor to the subject.

4. The method according to any one of claims 1 to 3, wherein the cancer cells are treated ex vivo with a chemotherapeutic agent or radiation and a TOP inhibitor to induce senescence resulting in TIS cells.

5. The method according to any one of claims 1 to 4, further comprising detection of a senescence-associated secretory phenotype (SASP) in the treated cells.

6. The method according to any one of claims 1 to 5, further comprising detecting the level of TOPcc in the cells wherein an increase in TOPcc levels is indicative of senescence in the cells.

7. The method according to any one of claims 1 to 6, wherein the treated cancer cells are assayed for SA-β-Gal to detect senescence.

8. The method according to any one of claims 1 to 7, wherein cells are treated ex vivo with an inhibitor of TOP1, TOP2, or both.

9. The method according to any one of claims 1 to 8, wherein the chemotherapeutic agent is cisplatin.

10. The method according to any one of claims 1 to 9, wherein the inhibitor of TOP1 is selected from irinotecan, camptothecin, and etoposide.

11. The method according to any one of claims 1 to 11, wherein the TIS cells are administered via a route selected from intravenous, intramuscular, subcutaneous, intraperitoneal, intradermal, intratumoral, intralesional and intraocular.

12. The method according to any one of claims 1 to 10, wherein said TIS cells home to any remaining cancer cells in the subject, and release cytokines and/or chemokines, thereby activating immune response.

13. The method according to any one of claims 1 to 12, wherein the cancer is ovarian cancer or melanoma.

14. The method according to any one of claims 1 to 13, wherein the checkpoint inhibitor is selected from a PD-1 or PD-L1 inhibitor or CTLA4 inhibitor.

15. The method according to any one of claims 1 to 14, wherein the TIS cells and the immune checkpoint inhibitor are administered in a coordinated therapeutic regimen.

16. The method according to any one of claims 1 to 15, wherein the TIS cells and the immune checkpoint inhibitor are administered sequentially.

17. The method according to any one of claims 1 to 15, wherein the TIS cells and the immune checkpoint inhibitor are administered simultaneously.

18. The method according to any one of claims 1 to 17 wherein the routes of administration for TIS cells and the immune checkpoint inhibitor are the same.

19. The method according to any one of claims 1 to 17, wherein the routes of administration for the TIS cells and the immune checkpoint inhibitor are different.

20. The method according to any one of claims 1 to 19, further comprising administering a chemotherapeutic agent to the subject.

21. The method according to any one of claims 1 to 20, wherein the subject has a chemotherapy-resistant cancer.

22. The method according to claim 21, wherein the chemotherapy-resistant cancer cells are sensitized via treatment with the TIS cells.

22. A pharmaceutical composition comprising TIS cells and a carrier, excipient, adjuvant or diluent.

23. The pharmaceutical composition according to claim 22, further comprising an immune checkpoint inhibitor.

24. A pharmaceutical composition produced by the following method:
a) obtaining cancer cells from a subject;
b) treating the cancer cells ex vivo with a chemotherapeutic agent, an inhibitor of TOP1 and/or an inhibitor of TOP2 to produce therapy induced senescent (TIS) cells; and
c) optionally, confirming senescence and/or sorting senescent cells from non-senescent cells.

25. The composition according to any one of claims 22 to 24 for use in the treatment of cancer.
